(19) United States
(12) Patent Application Publication
(10) Pub. No.: US 2019/0307771 A1
(43) Pub. Date: Oct. 10, 2019

(54) METHODS AND COMPOSITIONS FOR THE TREATMENT OF CANCER AND METABOLIC DISEASES

(71) Applicant: THE REGENTS OF THE UNIVERSITY OF CALIFORNIA, Oakland, CA (US)

(72) Inventor: Yu-Jui Yvonne Wan, Davis, CA (US)

(73) Assignee: THE REGENTS OF THE UNIVERSITY OF CALIFORNIA, Oakland, CA (US)

(21) Appl. No.: 16/406,916

(22) Filed: May 8, 2019

Related U.S. Application Data
(63) Continuation of application No. PCT/US2017/061198, filed on Nov. 10, 2017.
(60) Provisional application No. 62/421,156, filed on Nov. 11, 2016.
(60) Provisional application No. 62/489,929, filed on Apr. 25, 2017.

(51) Int. Cl. A61K 31/565; A61K 31/202; A61K 31/167; A61K 31/713; A61K 31/718; A61K 31/732; A61K 38/28; A61P 35/00

(52) U.S. Cl. CPC A61K 31/565; A61K 31/202; A61K 31/167; A61K 31/713; A61K 47/32; A61K 31/732; A61K 38/28; A61P 35/00; A61K 31/718

(57) ABSTRACT

The invention provides methods and pharmaceutical compositions for treating cancer or a metabolic disease in a subject. In some aspects, the invention comprises administering to the subject a therapeutically effective amount of a retinoid and a histone deacetylase (HDAC) inhibitor. In some embodiments, the HDAC inhibitor is a short-chain fatty acid and/or suberanilohydroxamic acid (SAHA). In other aspects, the invention comprises administering to the subject a therapeutically effective amount of a microRNA. Pharmaceutical compositions and kits for treating a subject with cancer or a metabolic disease are also provided herein.

### CROSS-REFERENCE TO RELATED APPLICATIONS

This application is a continuation of International Patent Application No. PCT/US2017/061198, filed Jun. 21, 2017, which claims priority to U.S. Provisional Application No. 62/421,156, filed Nov. 11, 2016, and U.S. Provisional Application No. 62/489,929, filed Apr. 25, 2017, the disclosures of which are herein incorporated by reference in their entirety for all purposes.

### BACKGROUND OF THE INVENTION

Despite advances in therapy over the years, cancer remains a prominent medical problem and is one of the leading causes of death worldwide. In 2012, there were approximately 14 million new cases of cancer, and approximately 8.2 deaths caused by cancer worldwide. It is expected that the number of new cases of cancer will increase from approximately 14 million in 2012 to approximately 30 million by the year 2030.

Furthermore, metabolic disease such as diabetes, obesity, non-alcoholic steatohepatitis (NASH) and non-alcoholic fatty liver disease (NAFLD) pose prominent threats to health worldwide, and are expected to continue to become more prominent. In 2015, nearly 10% of the American population had diabetes. In addition, more than one-third of American adults have obesity.

Accordingly, there is a need for new treatments for cancer and metabolic diseases such as diabetes, obesity, and fatty liver syndromes. The present invention satisfies this need, and provides related advantages as well.

### BRIEF SUMMARY OF THE INVENTION

In one aspect, methods for treating cancer in a subject are provided, the methods comprising administration of a therapeutically effective amount of a retinoid and a histone deacetylase (HDAC) inhibitor) to the subject.

In a second aspect, methods for treating a metabolic disease in a subject are provided, the methods comprising administration of a therapeutically effective amount of a retinoid and a histone deacetylase (HDAC) inhibitor to the subject.

In some embodiments, the HDAC inhibitor is selected from the group consisting of a short-chain fatty acid (SCFA), suberanilohydroxamic acid (SAHA), and a combination thereof. In some embodiments, the retinoid is selected from the group consisting of retinoic acid (RA), retinol, retinal, isotretinoin, alltretinoin, etretinate, acitretin, tazarotene, bexarotene, adapalene, seletinoid G, a retinyl ester, fenretinide, derivatives thereof, and a combination thereof. In some embodiments, the retinyl ester is selected from the group consisting of retinyl acetate, retinyl butyrate, retinyl propionate, retinyl palmitate, and a combination thereof. In particular embodiments, the retinoid is RA.

In some embodiments, the SCFA is selected from the group consisting of formate, acetate, propionate, butyrate, isobutyrate, valerate, isovalerate, and a combination thereof. In some embodiments, the HDAC inhibitor is SAHA. In some embodiments, the SCFA is butyrate. In some embodiments, the SCFA is propionate. In some embodiments, the SCFA is valerate. In some embodiments, the retinoid and the HDAC inhibitor are administered orally.

In a third aspect, methods for treating cancer in a subject are provided, the methods comprising administration of a therapeutically effective amount of a microRNA (miR) or a mimic thereof to the subject, wherein the miR is miR-22 and/or miR-34a. In some embodiments, the miR-22 comprises a nucleotide sequence having at least 90% sequence identity to SEQ ID NO:1. In some embodiments, the miR-34a comprises a nucleotide sequence having at least 90% sequence identity to SEQ ID NO:2. In some embodiments, the miR is virally expressed.

In some embodiments, liver cancer is treated in the subject. In some embodiments, colon cancer is treated in the subject. In some embodiments, the subject has one or more colon polyps.

In some embodiments, administration of the retinoid and HDAC inhibitor to the subject improves one or more symptoms of cancer in the subject. In some embodiments, the administration of the miR or the mimic thereof to the subject improves one or more symptoms of cancer in the subject.

In some embodiments, the metabolic disease that is treated in the subject is selected from the group consisting of non-alcoholic steatohepatitis (NASH), non-alcoholic fatty liver disease (NAFLD), diabetes, obesity, and a combination thereof.

In some embodiments, a sample is obtained from the subject. In some embodiments, the sample comprises blood, tissue, or a combination thereof. In some embodiments, the sample comprises normal tissue. In some embodiments, the sample comprises diseased tissue. In some embodiments, the sample comprises cancer tissue.

For methods of treating cancer in a subject according to methods of the present invention, in some embodiments, the level of one or more biomarkers is measured in the sample. In some embodiments, at least one of the one or more biomarkers is a microRNA (miR). In particular embodiments, the miR is selected from the group consisting of miR-22, miR-34a, and a combination thereof.

For methods of treating a metabolic disease in a subject according to methods of the present invention, in some embodiments, the level of one or more biomarkers is measured in the sample. In some embodiments, the one or more biomarkers is selected from the group consisting of a miR, FGF21, FGFR1c, Beta-klotho, blood glucose, aspartate aminotransferase (AST), alanine aminotransferase (ALT), the ratio of AST to ALT, gamma-glutamyl transferase (GGT), the aspartate to platelet ratio index (APRI), alkaline phosphatase (AP), bilirubin, ferritin, alpha-smooth muscle actin (αSMA), procollagen α1 (procol1), transforming growth factor-β (TGFβ), monocyte chemoattractant protein-1 (MCP1), interleukin-1β (IL-1b), tumor necrosis factor alpha (TNFα), connective tissue growth factor (CTGF), platelet derived growth factor receptor beta (PDGFRβ), and a combination thereof. In some embodiments, the miR is selected from the group consisting of miR-22, miR-34a, and a combination thereof.

In some embodiments, the measured level of the one or more biomarkers in the sample is abnormal compared to a reference sample. In some embodiments, the reference sample is obtained from the subject. In some embodiments, the reference sample is obtained from a different subject or a population of subjects. In some embodiments, the level of the one or more biomarkers is measured before the retinoid and HDAC inhibitor are administered to the subject. In other embodiments, the level of the one or more biomarkers is measured before the miR or mimic thereof is administered to the subject.

In some embodiments, the administration of the retinoid and HDAC inhibitor to the subject improves one or more symptoms of cancer in the subject. In some embodiments, the administration of the miR or the mimic thereof to the subject improves one or more symptoms of cancer in the subject. In some embodiments, the administration of the retinoid and HDAC inhibitor to the subject improves one or more symptoms of the metabolic disease in the subject.

In some embodiments, the methods of treating cancer or treating a metabolic syndrome in the subject further comprise administering a starch to the subject. In some embodiments, the method further comprises administering a probiotic agent and/or a prebiotic agent to the subject. In some embodiments, the probiotic comprises a bacterium that produces an SCFA. In some embodiments, the prebiotic comprises apple pectin, an inulin, or a combination thereof.

In some embodiments, the method further comprises administering a delivery-enhancing agent to the subject. In some embodiments, the delivery-enhancing agent is selected from the group consisting of a cyclodextrin, a hepatitis E virus-like particle, an inactivated yeast, an inactivated bacterium, polyvinyl acetate (PVA), an inulin or an ester thereof, and a combination thereof. In some embodiments, the HDAC inhibitor and the retinoid are packaged into PVA at a HDAC inhibitor-retinoid ratio of about 1:50 to about 1:1,000 by weight. In some embodiments, the inulin ester is selected from the group consisting of an inulin butyrate ester, an inulin propionate ester, and a combination thereof.

In another aspect, pharmaceutical compositions comprising a retinoid, an HDAC inhibitor, and a pharmaceutically acceptable carrier are provided. In some embodiments, the HDAC inhibitor is selected from the group consisting of a short-chain fatty acid (SCFA), suberanilohydroxamic acid (SAHA), and a combination thereof. In some embodiments, the retinoid is selected from the group consisting of retinoic acid (RA), retinol, retinal, isotretinoin, alltretinoin, etretinate, acitretin, tazarotene, bexarotene, adapalene, seletinoid G, a retinyl ester, fenretinide, derivatives thereof, and a combination thereof. In some embodiments, the retinyl ester is selected from the group consisting of retinyl acetate, retinyl butyrate, retinyl propionate, retinyl palmitate, and a combination thereof.

In some embodiments, the retinoid is RA. In particular embodiments, the concentration of RA is about 10 μM. In some embodiments, the SCFA is selected from the group consisting of formate, acetate, propionate, butyrate, isobutyrate, valerate, isovalerate, and a combination thereof. In some embodiments, the HDAC inhibitor is SAHA. In particular embodiments, the concentration of SAHA is about 5 μM. In some embodiments, the SCFA is butyrate. In particular embodiments, the concentration of butyrate is about 5 mM. In some embodiments, the SCFA is propionate. In particular embodiments, the concentration of propionate is about 10 mM. In some embodiments, the SCFA is valerate. In particular embodiments, the concentration of valerate is about 10 mM.

In yet another aspect, pharmaceutical compositions comprising a miR or a mimic thereof, wherein the miR is miR-22 and/or miR-34a, and a pharmaceutically acceptable carrier are provided. In some embodiments, the miR-22 comprises a nucleotide sequence having at least 90% sequence identity to SEQ ID NO:1. In some embodiments, the miR-34a comprises a nucleotide sequence having at least 90% sequence identity to SEQ ID NO:2.

In some embodiments, the pharmaceutical composition further comprises a starch. In some embodiments, the pharmaceutical composition further comprises a probiotic agent and/or a prebiotic agent. In some embodiments, the probiotic comprises a bacterium that produces an SCFA. In some embodiments, the prebiotic comprises apple pectin, an inulin, or a combination thereof.

In some embodiments, the pharmaceutical composition further comprises a delivery-enhancing agent. In some embodiments, the delivery-enhancing agent is selected from the group consisting of a cyclodextrin, a hepatitis E virus-like particle, an inactivated yeast, an inactivated bacterium, polyvinyl acetate (PVA), an inulin or an ester thereof, and a combination thereof. In some embodiments, the HDAC inhibitor and the retinoid are packaged into PVA at a HDAC inhibitor-retinoid ratio of about 1:50 to about 1:1,000 by weight. In some embodiments, the inulin ester is selected from the group consisting of an inulin butyrate ester, an inulin propionate ester, and a combination thereof. In some embodiments, the pharmaceutical composition comprises a nanoemulsion.

In some embodiments, the pharmaceutical composition is administered to the subject to treat cancer. In some embodiments, the cancer is liver cancer or colon cancer. In particular embodiments, the cancer is colon cancer and the subject has one or more colon polyps.

In some embodiments, the pharmaceutical composition is administered to the subject to treat a metabolic disease. In some embodiments, the metabolic disease is selected from the group consisting of non-alcoholic steatohepatitis (NASH), non-alcoholic fatty liver disease (NAFLD), diabetes, obesity, and a combination thereof.

In yet another aspect, kits for treating cancer and/or a metabolic disease in a subject are provided. In some embodiments, the kit comprises a pharmaceutical composition of the present invention. In some embodiments, the cancer is liver cancer or colon cancer. In some embodiments, the cancer is colon cancer and the subject has one or more colon polyps. In some embodiments, the metabolic disease is selected from the group consisting of non-alcoholic steatohepatitis (NASH), non-alcoholic fatty liver disease (NAFLD), diabetes, obesity, and a combination thereof. In some embodiments, the kit further comprises instructions for use.

### BRIEF DESCRIPTION OF THE DRAWINGS

FIGS. 1A-1C show that retinoic acid (RA) and butyrate synergistically promoted apoptosis and induced miR-22 and RARB expression. Data expressed as mean±SD (n=3). * denotes p<0.05 vs. DMSO control. # denotes p<0.05 vs. each individual treatment. FIG. 1A shows the effects of RA, butyrate, and/or suberanilohydroxamic acid (SAHA) on the viability of HCT116 colon cancer cells. The first bar represents the dimethyl sulfoxide (DMSO)-only negative vehicle control. FIG. 1B shows the expression of miR-22 in response to treatment of HCT116 cells with RA, butyrate, and/or SAHA. FIG. 1C shows the expression of RARB in response to treatment of HCT116 cells with RA, butyrate, and/or SAHA.

FIGS. 2A-2C show that RA and butyrate regulate miR-22 expression through RARβ. FIG. 2A shows a miR-22 promoter map (SEQ ID NOS:3-7, respectively). FIG. 2B shows the results of luciferase activity assays in cells that were transfected with PGL3 vectors containing one of the binding motif sequences depicted in FIG. 2A or PGL3 vector only. Cells were co-transfected with RARβ and RXRα or FXFR and RXRα and subsequently treated with DMSO only or a combination of RA and butyrate. FIG. 2C shows fold enrichment of DR5 and IR1 binding using an anti-RARβ antibody, compared to IgG, in HCT116 cells treated with RA, butyrate, and/or SAHA. DMSO-only was used as a negative control.

FIGS. 3A and 3B show that miR-22 targets HDAC4, SIRT1, and cyclin A2. FIG. 3A depicts alignments of nucleic acid sequences encoding Mmu-miR-22 (SEQ ID NO:1), Hsa-miR-22 (SEQ ID NO:1), HDAC4 (SEQ ID NO:9), SIRT1 (SEQ ID NO:9), and cyclin A2 (SEQ ID NO:10). FIG. 3B shows Western blots depicting protein levels of HDAC4, SIRT1, and cyclin A2 in HCT116 cells 48 hours after transfection with either a scramble control or an miR-22 mimic. β-actin expression was used as a reference.

FIGS. 4A-4C show that the reduction of HDAC and cyclin A2 by RA and butyrate was reversed with an miR-22 inhibitor. FIG. 4A shows a Western blot depicting HDAC4, SIRT1, and cyclin A2 expression in HCT116 cells after treatment with RA, butyrate, and/or SAHA. β-actin expression was used as a reference. FIG. 4B shows the relative expression of miR-22 in cells that were treated with a combination of RA and butyrate (or negative control) and transfected with either an miR-22 inhibitor or a scramble control. FIG. 4C shows a Western blot depicting expression of HDAC4, SIRT1, and cyclin A2 in cells that were treated with a combination of RA and butyrate (or negative control) and transfected with either an miR-22 inhibitor or a scramble control. β-actin expression was used as a reference.

FIG. 5 shows that RA and butyrate increased RARβ transcription through histone modification. Fold enrichment of HDAC4, SIRT1, and H3K9Ac, compared to IgG, was determined in cells that were treated with RA, butyrate, and/or SAHA.

FIG. 6 shows the effect of RA, the HDAC inhibitor SAHA, and/or various short-chain fatty acids on the viability of HCT116 and DLD-1 colon cancer cells. DMSO-only was used as a negative control. * denotes p<0.05 vs. DMSO control. # denotes a statistically significant difference (p<0.05) when compared to single-agent treatment.

FIGS. 7A-7D show that RA and short-chain fatty acids that have HDAC inhibitory effect induced miR-22 expression through the binding of RARβ to a DR5 motif. FIG. 7A shows the relative expression level of miR-22 in cells that were treated with RA, butyrate, propionate, valerate, and/or SAHA. FIG. 7B shows a map of putative regulatory binding motifs (SEQ ID NOS:3-7, respectively). FIG. 7C shows luciferase assay results. FIG. 7D shows chromatin immunoprecipitation data.

FIGS. 8A-8C show that miR-22 reduces protein deacetylases, including HDAC1. FIG. 8A depicts sequence alignments of two miR-22 variants (SEQ ID NO:1) and the 3′-untranslated region of HDAC1 (SEQ ID NO:11). FIG. 8B shows luciferase assay results. Data are presented as mean±SD. ** denotes p<0.01. FIG. 8C shows Western blot results illustrating protein expression levels of HDAC1, HDAC4, SIRT1, and a β-actin control.

FIGS. 9A-9E show that the inhibition of HDAC1, HDAC4, SIRT1, and cyclin A2 by RA and butyrate was reversed by miR-22 inhibitors. FIG. 9A shows a Western blot depicting protein expression of HDAC1, HDAC4, SIRT1, cyclin A2, and a β-actin control in HCT116 cells that were treated with RA and/or butyrate or SAHA. FIG. 9B depicts relative miR-22 expression levels in cells that were treated with an miR-22 inhibitor and/or a combination of RA and butyrate. FIG. 9C shows Western blot results depicting protein expression levels from the experiment described for FIG. 9B. FIG. 9D shows a plot of miR-22 expression in colon polyps (P), cancers (T), and adjacent normal tissue (N). FIG. 9E shows Western blots depicting protein expression of HDAC1, HDAC4, SIRT1, cyclin A2, and a β-actin control in colon cancer (T) and normal tissue (N) samples obtained from four patients.

FIGS. 10A-10E show that that the combination of RA and HDAC inhibitors synergistically promoted apoptosis and the induction of interaction of NUR77 and RARβ in HCT116 cells. Data are expressed as mean±SD (n=3). *p<0.05, **p<0.01, *p<0.005, vs. DMSO. # p<0.05 vs. single treatment. FIG. 10A shows cell viability in HCT116 cells that were treated with RA and/or butyrate, propionate, valerate, or SAHA. FIG. 10B shows relative mRNA levels of Nur77 and RARB in cells treated as described for FIG. 10A. FIG. 10C shows the induction and co-localization of NUR77 and RARβ in HCT116 cells. FIG. 10D shows the protein expression levels of NUR77, RARβ, cleaved Caspase 3, phospho JNK1/2, and total (T)-JNK1/2. FIG. 10E illustrates proteins extracted from HCT116 cells and immunoprecipitated by anti-NUR77 or anti-RARβ antibodies or IgG, followed by Western blot.

FIG. 11 shows ChIP data from cell lysates that illustrates the combination effects of RA plus HDAC inhibitors in reducing the recruitment of HDAC1, HDAC4, and SIRT1, as well as increasing the acetylation within the RARB and Nur77 genes.

FIGS. 12A-12C show that RA and butyrate-induced NUR77 acetylation was accompanied by Nur77 transcriptional regulatory activity. FIG. 12A shows the expression levels of proteins extracted from HCT116 cells. Proteins were immunoprecipitated with anti-NUR77 and anti-RARβ antibodies or IgG, followed by Western blot. FIG. 12B shows ChIP-qPCR data illustrating the binding of NUR77 and RARβ to their target genes. FIG. 12C shows the relative mRNA levels of BRE, RARB, CCND2, and CYP26A1. The data are presented as the means±S.D.

FIGS. 13A and 13B show that butyrate signaling is reduced in colon polyps and cancers. FIG. 13A shows the mRNA levels of short-chain fatty acid receptors GPR41, GPR43, and GPR109A in colon adenocarcinomas (T, n=20) or colon polyps (P, n=20) and their paired adjacent normal (N) specimens. FIG. 13B shows the abundance of butyrate-generating bacteria genes bcoA and buk in colon adenocarcinomas (T, n=10) or colon polyps (P, n=20) and their paired adjacent normal (N) specimens.

FIG. 14 shows mRNA levels of ALDH1A1 and SCFA receptor genes in human cancers (T) and their adjacent benign tissues (N). n=10 for HCCs, n=20 for colon cancers.

FIGS. 15A and 15B illustrate particular interactions between RA and SCFAs. FIG. 15A shows ileal and hepatic mRNA levels in C57BL/6 mice treated with RA (15 mg/kg body weight) and/or butyrate (500 mg/kg) by oral gavage every other day for one week. FIG. 15B shows blood glucose levels after insulin injection in mice treated with and without RA plus butyrate. N=4, mean+/−SD, * denotes p<0.05.

FIG. 16 shows repression of cyclin A2 upon CDCA treatment and miR-22 mimic transfection in liver Huh7 and colon HCT116 cells.

FIGS. 17A-17D illustrate particular interactions between miR-22 and cyclin A2. FIG. 17A shows ileal and liver miR-22 levels in wild type and FXR knockout mice. FIG. 17B shows ileal and liver cyclin A2 protein levels in wild type and FXR knockout mice. FIG. 17C shows Ki-67 staining of liver and colon sections of WT and FXR knockout mice (n>3). FIG. 17D shows miR-22 and cyclin A2 mRNA levels in liver and colon specimens. Specimens were derived from tumors and adjacent normal specimens from four patients. * denotes p<0.05, ** denotes p<0.01.

FIGS. 18A and 18B illustrate the synergistic effects of RA and HDAC inhibitors. FIG. 18A shows relative expression levels of miR-22 in HCT116 cells treated with RA and/or butyrate, propionate, valerate, or SAHA. FIG. 18B shows expression of HDAC1, HDAC4, SIRT1, and cyclin A2 in HCT116 cells treated with RA and/or butyrate or SAHA.

FIGS. 19A and 19B show the effect of miR-22 mimics on the levels of proteins in HCT116 cells. FIG. 19A shows the levels of HDAC1, HDAC4, SIRT1, SRC1, and cyclin A2 in Huh7 and HCT116 cells treated with a miR-22 mimic. FIG. 19B shows the levels of the same proteins as in FIG. 19A in HCT116 cells treated with miR-22 mimic or scramble control, with and without treatment with a combination of RA and butyrate.

FIG. 20 shows the levels of HDAC1, HDAC4, SIRT1, cyclin A2, p-ERK1/2, T-ERK1/2, and NUR-77 in liver tumors (T) and their adjacent normal tissues (N) from four patients who had liver cancer.

FIGS. 21A and 21B show the effects of RA and HDAC inhibitor treatment on liver and colon cancer cells. FIG. 21A shows the combination effect of fenretinide and HDAC inhibitors Scriptaid and TSA (trichostatin) in inducing caspase 3/7 activity in HepG2 liver cancer cells. FIG. 21B shows the combination effect of RA and SCFAs or SAHA or all three on the viability of HCT116 and DLD-1 colon cancer cells. Fenretinide (10 μM), RA (10 μM). SAHA (5 μM), butyrate (5 mM), propionate (10 mM), and valerate (10 mM) were used to treat the cells for 48 hours. * denotes p<0.05 vs. DMSO control, # denotes p<0.05 vs. single agent treatment.

FIGS. 22A-22C illustrate interactions between NUR77 and RARβ. FIG. 22A shows the interaction of NUR77 and RARβ regulated by fenretinide and HDAC inhibitors Scriptaid and TSA measured by immunoprecipitation followed by Western blot using Huh7 cells. FIG. 22B shows NUR77 is essential for fenretinide- and Scriptaid-induced apoptosis of Huh7 cells. Knockdown of NUR77 by siRNA prevented fenretinide- and Scriptaid-induced apoptosis of Huh7 cells. Cytosolic NUR77 and caspase 3 were co-localized. EGF-treated Huh7 cells had increased nuclear NUR77, but no cleaved caspase 3. FIG. 22C shows that the detection of cytosolic NUR77 was dependent on the presence of RARβ in fenretinide or Scriptaid-treated Huh7 cells. When RARβ expression was knocked down by siRNA, cytosolic NUR77 was no longer detectable.

FIGS. 23A-23D show the combination effect of RA and butyrate or SAHA in regulating NUR77 and RARβ. FIG. 23A shows the combination effect of RA and butyrate or SAHA in regulating the mRNA levels of NUR77 and RARβ in HCT116 cells. FIG. 23B shows the combination effect of RA and butyrate or SAHA in regulating the protein levels of NUR77 and RARβ in HCT116 cells. FIG. 23C shows EGF-induced nuclear NUR77. FIG. 23D shows RA/butyrate-induced cytosolic NUR77 and RARβ in HCT116 cells. Cells were treated with DMSO, RA (10 μM), butyrate (5 mM), SAHA (5 μM), or EGF (32 nM) for 48 hours.

FIG. 24 shows liver tumors that spontaneously developed in 15-month old FXR knockout mice.

FIGS. 25A and 25B illustrate the abundance of microbes in cecal contents. FIG. 25A shows the relative abundance of microbial families in the cecal content of 10-month old wild type (WT) and FXR knockout mice. FIG. 25B shows the relative abundance of bacterial genes in the cecal content of 10-month old wild type (WT) and FXR knockout mice. n=6. Data shown as mean+/−SD. * denotes p<0.05, ** denotes p<0.01.

FIG. 26 shows the morphology of mouse livers from Western diet-fed FXR knockout mice treated with and without (control) polymyxin B (100 mg/liter in drinking water) for three months. The treatment was started when mice were seven months old. Mice were sacrificed when they were ten months old. n>4.

FIG. 27 shows the relative FXR mRNA levels in cancers (T) and adjacent benign specimens (N) (liver, n=10, colon, n=20).

FIGS. 28A-28C show reduced miR-22 and impaired RA, SCFA, and protein deacetylase signaling in human colon polyps and cancers. FIG. 28A shows expression levels of miR-22, CCNA2, short-chain fatty acid receptors GPR41, GPR43, and GPR109A, and RARβ in human colon polyps (P, n=20) and adenocarcinoma (T, n=20) along with paired adjacent normal tissue (N, n=20). FIG. 28B shows abundance of butyrate-generating bacterial genes bcoA and buk in colon polyps (P, n=20) and adenocarcinomas (T, n=10) along with paired adjacent normal tissues (N, n=20, 10). FIG. 28C shows levels of indicated proteins in human colon adenocarcinomas (T) along with paired adjacent normal tissues (N) (n=4).

FIGS. 29A-29D show that RA and HDAC inhibitors induce miR-22 through RARβ. FIG. 29A shows miR-22 level in HCT116 cells after butyrate (5 mM), propionate (10 mM), valerate (10 mM), SAHA (5 μM), and/or RA (10 μM) treatment for 24 hours. * indicates p<0.05, ** p<0.01, *** p<0.0001 in treatment vs. DMSO control; # indicates p<0.05 in combined RA plus SCFA or SAHA vs. single treatment. FIG. 29B shows putative nuclear receptor binding sites (SEQ ID NOS:3-7, respectively) within 2 kb upstream from transcription start site of miR-22. FIG. 29C shows putative DR5, ER6, DR1, IR1, and DR3 motifs were each cloned into a PGL3 vector. HCT116 cells were co-transfected with RARβ and RXRα or FXR and RXRα expression plasmids along with indicated PGL3 reporter constructs. Six hours after transfection, cells were treated with RA and butyrate or DMSO for 24 hours. PGL3-Neg and PGL3-5×DR5 were used as negative and positive controls, respectively. FIG. 29D shows chromatin immunoprecipitation followed by qPCR (ChIP-qPCR) revealed the combination effect of butyrate (5 mM) or SAHA (5 μM) plus RA (10 μM) on the recruitment of RARβ to the DR5 and IR1 motifs.

FIGS. 30A-30E show that RA plus butyrate silencing of protein deacetylases is miR-22 dependent. FIG. 30A shows a psiCHECK2-HDAC1 construct containing the 3′UTR of HDAC1 was co-transfected with either miR-22 mimics or miR-22 inhibitors into HCT116 cells. Scramble constructs were used as negative controls. Data are presented as mean±SD with ** indicating p<0.01. FIG. 30B shows HDAC1, HDAC4, and SIRT1 protein levels using cell lysates from HCT116 cells transfected with miR-22 mimics or scramble controls. FIG. 30C shows that miR-22 (SEQ ID NO:1) partially pairs with the 3′UTR of the HDAC1 (SEQ ID NO:11), HDAC4 (SEQ ID NO:9) and SIRT1 (SEQ ID NO:9); miR-22 sequence is conserved between humans and mice. FIG. 30D shows levels of indicated proteins in butyrate (5 mM), SAHA (5 μM), and/or RA (10 μM)-treated HCT116 cells for 24 hours. FIG. 30E levels of indicated proteins in RA plus butyrate treated-HCT116 cells 48 hours after transfection with miR-22 inhibitors or scramble controls.

FIGS. 31A-31E show that RA plus HDAC inhibitors promote apoptosis via cytosolic NUR77 and RARβ induction in HCT116 cells. FIG. 31A shows cell viability and mRNA levels of NUR77 and RAM in HCT116 cells treated with RA, SCFAs, and SAHA for 48 hours. Data are expressed as mean±SD (n=3) * indicates p<0.05, ** p<0.01, *** p<0.001 in treatment vs. DMSO; # indicates p<0.05 in combined RA plus SCFA or SAHA vs. single treatment. FIG. 31B shows induction and localization of NUR77 and RARβ in HCT116 cells treated with RA plus butyrate or SAHA. Treated cells were immunostained with anti-NUR77 and anti-RARβ antibodies followed by Alexa Fluor secondary antibodies. FIG. 31C shows protein levels of NUR77, RARβ, cleaved caspase 3, phosphorylated (P)-JNK1/2, and total (T)-JNK1/2 in HCT116 cells in response to RA, butyrate, or SAHA treatment. FIG. 31D shows NUR77 and RARβ interaction in HCT116 cells in response to RA, butyrate, and SAHA treatment. Protein extracts were immunoprecipitated using anti-NUR77, anti-RARβ, or IgG antibody followed by Western blot with anti-RARβ and or anti-NUR77 antibody. FIG. 31E shows induction and localization of NUR77 in RA plus butyrate-treated HCT116 cells infected with adeno-scramble control-GFP or adeno-miR-22 inhibitor-GFP virus. At 48 hours post adenoviral infection, cells were treated with RA (10 μM) plus butyrate (5 mM) or DMSO for 24 hours. Treated cells were immunostained with anti-NUR77 antibody followed by Alexa Fluor secondary antibody.

FIGS. 32A-32D show that RA plus HDAC inhibitors reduce HDAC1, HDAC4, and SIRT1 recruitment while increasing histone acetylation of NUR77 and RARβ genes as well as protein acetylation of both nuclear receptors. FIG. 32A shows HCT116 cells were treated with RA (10 μM), butyrate (5 mM), or SAHA (5 μM) for 24 hours. ChIP was performed on cell lysates using anti-HDAC1, anti-HDAC4, anti-SIRT1, or anti-acetylated H3K9 antibody followed by qPCR using primers specific for the NUR77 and RAM genes. Binding is expressed relative to IgG antibody negative control. FIG. 32B shows RA plus butyrate treatment or adeno-miR-22 infection induced protein acetylation of NUR77 and RARβ as revealed by immunoprecipitation followed by Western blot. Protein extracts from treated-HCT116 cells were immunoprecipitated using anti-NUR77, anti-RARβ or IgG antibody followed by Western blot using anti-acetyl lysine antibody. FIGS. 32C and 32D show ChIP-qPCR data showing NUR77 and RARβ binding to their target genes; mRNA levels of NUR77 target genes BRE and CCND2 and RARβ target genes RAM and CYP26A1 in RA, butyrate, or SAHA-treated HCT116. Data are presented as the mean±S.D.

FIGS. 33A-33C show that RA plus butyrate reduce tumor size and induce miR-22-mediated silencing of protein deacetylases in mouse xenograft model. FIG. 33A shows volume and weight of HCT116-generated tumors in control and RA plus butyrate-treated mice; representative tumors seen in control and treatment groups. FIGS. 33B and 33C show RNA and protein levels in tumors generated from HCT116 cells. Data are presented as the mean±S.D with ** indicating p<0.01, ***p<0.0001 (n=16).

FIGS. 34A-34C show that adenoviral expression of miR-22 reduces tumor size and silences protein deacetylases in mouse xenograft model. FIG. 34A shows volume and weight of HCT116 cell-generated tumors in mice receiving either adeno-scramble control or adeno-miR-22; representative tumors seen in control and treatment groups. FIGS. 34B and 34C show RNA and protein levels in tumors generated from HCT116 cells. Data are presented as the mean±S.D with ** indicating p<0.01, *** p<0.0001, and n=16.

### DETAILED DESCRIPTION OF THE INVENTION

### I. Introduction

The present invention is based, in part, on the discovery that retinoids and short-chain fatty acids can induce microRNA (miR) activity, and further that retinoids and short-chain fatty acids can inhibit histone deacetylase (HDAC) activity, thus inhibiting cancer cell growth. The present invention is also based, in part, on the surprising discovery that retinoids and HDAC inhibitors, when combined, have an ability to induce miR activity and inhibit cancer cell viability that is greater than either retinoids or HDAC inhibitors alone.

### II. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.”

The term “cancer” refers to any of various malignant neoplasms characterized by the proliferation of anaplastic cells that tend to invade surrounding tissue and metastasize to new body sites. Non-limiting examples of different types of cancer suitable for treatment using the method and compositions of the present invention include colorectal cancer, colon cancer, anal cancer, liver cancer, ovarian cancer, breast cancer, lung cancer, bladder cancer, thyroid cancer, pleural cancer, pancreatic cancer, cervical cancer, prostate cancer, testicular cancer, bile duct cancer, gastrointestinal carcinoid tumors, esophageal cancer, gall bladder cancer, rectal cancer, appendix cancer, small intestine cancer, stomach (gastric) cancer, renal cancer (i.e., renal cell carcinoma), cancer of the central nervous system, skin cancer, oral squamous cell carcinoma, choriocarcinomas, head and neck cancers, bone cancer, osteogenic sarcomas, fibrosarcoma, neuroblastoma, glioma, melanoma, leukemia (e.g., acute lymphocytic leukemia, chronic lymphocytic leukemia, acute myelogenous leukemia, chronic myelogenous leukemia, or hairy cell leukemia), lymphoma (e.g., non-Hodgkin's lymphoma, Hodgkin's lymphoma, B-cell lymphoma, or Burkitt's lymphoma), and multiple myeloma.

The term “metabolic disease” refers to any disease or disorder that disrupts normal metabolism, including any disease that disrupts or dysregulates biochemical reactions that function to convert food into energy, process or transport amino acids, proteins, carbohydrates (e.g., sugars, starches), or lipids (e.g., fatty acids), etc. In some embodiments, a metabolic disease results in the abnormal processing or regulation of sugars, lipids, cholesterol, and/or the metabolism of drugs (e.g., by the liver). Non-limiting examples of metabolic diseases include obesity, insulin resistance, type 2 diabetes, hyperlipidemia, non-alcoholic fatty liver disease (NAFLD), and non-alcoholic steatohepatitis (NASH), as well as the sequelae of such diseases.

The term “retinoid” refers to a class of compounds that are vitamers of vitamin A (i.e., compounds that generally have a similar structure to vitamin A) or are chemically related to vitamin A. Retinoids include, any natural or synthetic derivative of retinol.

The term “histone deacetylase” or “HDAC” refers to a class of enzymes (Enzyme Commission number 3.5.1.98) that remove acetyl groups from proteins, including ε-N-acetyl lysine amino acids on histones. Histone deacetylation allows histones to wrap and compact DNA more tightly within chromatin, which is associated with gene silencing. Class I HDACs include HDAC1, HDAC2, HDAC3, and HDAC8. Class IIA HDACs include HDAC4, HDAC5, HDAC7, and HDAC9. Class IIB HDACs include HDAC6 and HDAC10. Class III HDACs include SIRT1, SIRT2, SIRT3, SIRT4, SIRT5, SIRT6, and SIRT7 in mammals and Sir2 in yeast. Class IV HDACs include HDAC11.

In humans, HDAC1 is encoded by the HDAC1 gene. A non-limiting example of a human HDAC1 amino acid sequence is set forth under GenBank Accession number NM_004964.2→NP_004955.2. In humans, HDAC4 is encoded by the HDAC4 gene. A non-limiting example of a human HDAC4 amino acid sequence is set forth under GenBank Accession number NM_006037.3→NP_006028.2. In humans SIRT1 is encoded by the SIRT1 gene. Non-limiting examples of human SIRT1 amino acid sequences are set forth under GenBank Accession number NM_001142498.1→NP_001135970.1, NM_001314049.1→NP_001300978.1, and NM_012238.4→NP_036370.2.

The term “histone deacetylase inhibitor” of “HDAC inhibitor” refers to any natural or synthetic compound or agent that decreases or suppresses the activity and/or expression of an HDAC. In some embodiments, an HDAC inhibitor decreases or suppresses the mRNA expression of an HDAC (e.g., transcription from a gene encoding an HDAC is decreased or suppressed). In some embodiments, an HDAC inhibitor decreases or suppresses the protein expression of an HDAC (e.g., translation of an mRNA expressed from an HDAC gene is decreased or suppressed). In some embodiments, an HDAC inhibitor decreases or suppresses the enzymatic activity of an HDAC. In some embodiments, an HDAC inhibitor decreases or suppresses the ability of an HDAC to deacetylate a protein, e.g., a histone.

Non-limiting examples of HDAC inhibitors include suberanilohydroxamic acid (SAHA), short-chain fatty acids (e.g., formate, acetate, propionate, butyrate, isobutyrate, valerate, isovalerate), entinostat, panobinostat, trichostatin A, Scriptaid, mocetinostat, chidamide, TMP195, citarinostat, belinostat, depsipeptide, MC1568, tubastatin, givinostat, dacinostat, CUDC-101, JNJ-26481585, pracinostat, PCI-34051, PCI-34051, droxinostat, abexinostat, RGFP966, AR-42, ricolinostat, valproic acid, tacedinaline, CUDC-907, curcumin, M344, tubacin, RG2833, resminostat, divalproex, sodium phenylbutyrate, TMP269, CAY10683, tasquinimod, BRD73954, splitomicin, HPOB, LMK-235, nexturastat A, (−)-parthenolide, CAY10603, 4SC-202, BG45, and ITSA-1.

The term “microRNA” or “miR” refers to a small non-coding RNA molecule (e.g., containing about 22 nucleotides) found in plants, animals, and some viruses, that functions in RNA silencing and post-transcriptional regulation of gene expression. Non-limiting examples are miR-22 (SEQ ID NO:1) and miR-34a (SEQ ID NO:2).

The terms “subject,” “individual,” and “patient” are used interchangeably herein to refer to a vertebrate, preferably a mammal, more preferably a human. Mammals include, but are not limited to, murines, rats, simians, humans, farm animals, sport animals, and pets. Tissues, cells and their progeny of a biological entity obtained in vivo or cultured in vitro are also encompassed.

The term “nanoemulsion” refers to a colloidal particulate system in the submicron size range. Nanoemulsions are particularly useful for acting as carriers in drug molecule delivery. Sizes range from about 10 nm to about 1,000 nm (e.g., about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 nm).

As used herein, the term “administering” includes oral administration, topical contact, administration as a suppository, intravenous, intraperitoneal, intramuscular, intralesional, intrathecal, intranasal, intraosseous, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, intraosseous, and intracranial. Other modes of delivery not limited to, the use of liposomal formulations, intravenous infusion, transdermal patches, etc.

The term “treating” refers to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. Therapeutic benefit can also mean to effect a cure of one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “therapeutically effective amount” or “sufficient amount” refers to the amount of a retinoid, HDAC inhibitor, or composition that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific amount may vary depending on one or more of: the particular agent chosen, the target cell type, the location of the target cell in the subject, the dosing regimen to be followed, whether it is administered in combination with other compounds, timing of administration, and the physical delivery system in which it is carried.

For the purposes herein an effective amount is determined by such considerations as may be known in the art. The amount must be effective to achieve the desired therapeutic effect in a subject suffering from cancer or a metabolic disease. The desired therapeutic effect may include, for example, improvement in or amelioration of undesired symptoms associated with cancer or the metabolic disease, prevention of the manifestation of such symptoms before they occur, slowing down the progression of symptoms associated with cancer or the metabolic disease, slowing down or limiting any irreversible damage caused by the cancer or the metabolic disease, lessening the severity of or curing cancer or the metabolic disease, or improving the survival rate or providing more rapid recovery from cancer or the metabolic disease. Further, in the context of prophylactic treatment the amount may also be effective to prevent the development of the cancer or the metabolic disease.

The term “pharmaceutically acceptable carrier” refers to a substance that aids the administration of an active agent to a cell, an organism, or a subject. “Pharmaceutically acceptable carrier” refers to a carrier or excipient that can be included in the compositions of the invention and that causes no significant adverse toxicological effect on the patient. Non-limiting examples of pharmaceutically acceptable carrier include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g. antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor, etc. In some instances, the carrier is an agent that facilitates the delivery of a retinoid, HDAC inhibitor, or composition to a target cell or tissue. One of skill in the art will recognize that other pharmaceutical carriers are useful in the present invention.

The term “nucleic acid” as used herein refers to a polymer containing at least two deoxyribonucleotides or ribonucleotides in either single- or double-stranded form and includes DNA, RNA, and hybrids thereof. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. RNA may be in the form of small interfering RNA (siRNA), Dicer-substrate dsRNA, small hairpin RNA (shRNA), asymmetrical interfering RNA (aiRNA), microRNA (miRNA), mRNA, tRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “prodrug” means a medication or compound that, after administration, is converted to a biologically or pharmacologically active form. Conversion of the prodrug to the active compound is typically the result of metabolism within the body. As a non-limiting example, tributyrin is a prodrug of butyric acid, of which butyrate is the conjugate base.

The term “alpha-smooth muscle actin or “αSMA” refers to a protein that is encoded by the ACTA2 gene, and is commonly used as a marker of myofibroblast formation. αSMA belongs to the actin family of proteins, which are highly conserved and play roles in cell motility, structure, and integrity. The gene encoding αSMA is also known as AAT6, ACTSA, or MYMY5, and is located on human chromosome 10. Non-limiting examples of αSMA amino acid sequences are set forth in GenBank Accession Nos. NM_001141945.2→NP_001135417.1 (human), NM_001613.2→NP_001604.1 (human), and NM_007392.3→NP_031418.1 (mouse).

The term “monocyte chemoattractant protein-1” or “MCP1” refers to a small cytokine that belongs to the CC chemokine family and is encoded by the CCL2 gene. MCP1 is also known as chemokine (C—C motif) ligand 2 (CCL2) or small inducible cytokine A2. The CCL2 gene is found on chromosome 17 in humans. MCP1 recruits monocytes, memory T cells, and dendritic cells to sites of inflammation. Non-limiting examples of MCP1 amino acid sequences are set forth in GenBank Accession Nos. NM_002982.3→NP_002973.1 (human) and NM_011331.2→NP_035461.2 (mouse).

The term “procollagen α1” or “procol1” refers to a protein encoded by the COL1A1 gene. A chain of procollagen α1 combines with a second chain of procollagen α1 and one chain of procollagen α2 (encoded by the COL1A2 gene) to form type I procollagen, which is then processed into type I collagen. Type I collagen is a fibrillary collagen found in most connective tissues in the body, including cartilage. Procol1 expression is associated with the development of fibrosis. Non-limiting examples of procol1 amino acid sequences are set forth in GenBank Accession Nos. NM_000088.3→NP_000079.2 (human) and NM_007742.4→NP_031768.2 (mouse).

The term “interleukin-1β” or “IL-1b” refers to a cytokine that is also known as leukocytic pyrogen, mononuclear cell factor, lymphocyte activating factor, IL1B, IL-1, IL1-BETA, or IL1F2, and is encoded by the IL1B gene. IL-1b plays roles in the inflammatory response and is involved in cell proliferation, differentiation, and apoptosis, as well as the development of fibrosis.

The term “transforming growth factor-β” or “TGFβ” refers to a multifunctional cytokine of the transforming growth factor superfamily that includes three different isoforms (TGFβ-1, TGFβ-2, and TGFβ-3. The three isoforms are encoded by the genes TGFB1, TGFB2, and TGFB3, respectively. TGFβ plays roles in cell proliferation, wound healing, and synthesis of extracellular matrix molecules. TGFβ is associated with the development of fibrosis in many different organs via its promotion of mesenchymal cell proliferation, migration, and accumulation following an inflammatory response.

The term “tumor necrosis factor alpha” or “TNFα” refers to the cytokine encoded by the gene TNF (also known as TNFA). TNFα is also known as tumor necrosis factor, TNF, TNFA, DIF, TNFSF2, cachexin or cachectin. TNFα is involved in systemic inflammation and is one of the cytokines that comprise the acute phase reaction. TNFα is produced primarily by activated macrophages, but is also produced by CD4+ lymphocytes, natural killer cells, neutrophils, mast cells, eosinophils, and neurons. Non-limiting examples of TNFα amino acid sequences are set forth in GenBank Accession Nos. NM_000594.3→NP_000585.2 (human) and NM_001278601.1→NP_001265530.1 (mouse).

The term “connective tissue growth factor” or “CTGF” refers to the matricellular protein of the CCN family of extracellular matrix-associated heparin binding proteins that is encoded by the CTGF gene. CTGF is also known as CCN2, HCS24, IGFBP8, or NOV2. Non-limiting examples of CTGF amino acid sequences are set forth in GenBank Accession Nos. NM_001901.2→NP_001892.1 (human) and NM_010217.2→NP_034347.2 (mouse). CTGF is associated with virtually all fibrotic pathology, in addition to wound healing. It has also been shown that CTGF cooperates with TGFβ to promote sustained fibrosis.

The term “platelet derived growth factor receptor beta” or “PDGFRβ” refers to the beta form of the platelet derived growth factor receptor that is encoded by the gene PDGFRB. Platelet derived growth factor receptor beta is also known as PDGFRB, CD140B, IBGC4, IMF1, JTK12, PDGFR, PDGFR-1, PDGFR1, KOGS, or PENTT. PDGFRβ is a cell surface tyrosine kinase receptor that, when activated following binding of a PDGF ligand and subsequent dimerization with another PDGFR beta receptor or a PDGFR alpha receptor, activates cellular signaling pathways that play roles in cell proliferation, differentiation, and growth. A non-limiting example of a PDGFRβ amino acid sequence is set forth in GenBank Accession No. NM_002609.3→NP_002600.1. PDGFRβ activation is associated with the replication, survival and migration of myofibroblasts during the progression of fibrotic diseases.

The term “aspartate aminotransferase” or “AST” refers to a pyridoxal phosphate (PLP)-dependent transaminase enzyme (Enzyme Commission number 2.6.1.1) that is also known as aspartate transaminase, AspAT, ASA, AAT, or serum glutamic oxaloacetic transaminase (SGOT). AST plays important roles in amino acid metabolism, catalyzing the transfer of alpha amino groups between aspartate and glutamate. AST is a common biochemical marker of liver disease, as it is released from liver cells following liver injury, manifesting as elevated AST concentrations when measured using a blood test. Normal AST reference ranges for blood tests are 8-40 IU/L for males and 6-34 IU/L for females. The ratio of AST to ALT is also a common clinical biomarker for liver disease.

The term “alanine aminotransferase” or “ALT” refers to a transaminase enzyme (Enzyme Commission number 2.6.1.2) that is also known as alanine transaminase, serum glutamate-pyruvate transaminase (SGPT), or serum glutamic-pyruvate transaminase (SGPT). ALT catalyzes the transfer of an amino group from L-alanine to α-ketoglutarate and plays important roles in the alanine cycle. ALT is a common biochemical marker of liver disease, as it is released from liver cells following liver injury, manifesting as elevated ALT concentrations when measured using a blood test. Normal ALT reference ranges for blood tests are <52 IU/L for males and <34 IU/L for females. The ratio of AST to ALT is also a common clinical biomarker for liver disease.

The term “AST to platelet ratio index” or “APRI” refers to a method of using a subject's AST level, as measured using a blood test, and the subject's platelet count to predict the amount of liver fibrosis in the subject, as non-invasive alternative to liver biopsy. APRI is calculated using the following formula:

$$A P R I = \frac{\frac{A S T level}{A S T upper limit of normal}}{platelet count} \times 100$$

wherein AST level and AST upper limit of normal are expressed in units of IU/L and platelet count is expressed in units of 10⁹/L. A commonly-recommended value of AST upper limit of normal is 40 IU/L. Higher APRI values are associated with greater positive predictive values of liver fibrosis.

The term “gamma-glutamyl transferase” or “GGT” refers to an enzyme that transfers gamma-glutamyl functional groups and is also known as gamma-glutamyl transpeptidase, GGTP or gamma-GT (Enzyme Commission number 2.3.2.2). GGT catalyzes the transfer of the gamma-glutamyl moiety of glutathione to acceptors that include amino acids, peptides, and water (i.e., the formation of glutamate), and plays a role in the gamma-glutamyl cycle, which functions in glutathione degradation and drug detoxification. GGT is useful for determining whether an increase in alkaline phosphatase is due to skeletal disease (in which case GGT levels will be normal) or liver disease (in which case GGT will be elevated).

The term “alkaline phosphatase” or “AP” or “ALP” refers to the hydrolase enzyme (Enzyme Commission number 3.1.3.1) that is also known as alkaline phosphomonoesterase, phosphomonoesterase, glycerophosphatase, alkaline phosphohydrolase, alkaline phenyl phosphatase, or orthophosphoric-monoester phosphohydrolase (alkaline optimum). AP removes phosphate groups from many different molecules, including nucleotides, proteins, and alkaloids. When liver cells are damaged, AP is released, thus elevated levels of AP in blood tests can be indicative of liver disease.

The term “bilirubin” refers to the yellow breakdown product of normal heme catabolism, and has a chemical formula of C₃₃H₃₆N₄O₆ and a molar mass of 584.67 g/mol. Measurement of bilirubin can be “indirect” (i.e., unconjugated bilirubin) or “direct” (i.e., conjugated bilirubin). Normal bilirubin levels, when measured using a blood test, range between 0 and 0.3 mg/dl for conjugated bilirubin, and 0.3 to 1.9 mg/dl for total bilirubin (i.e., conjugated and unconjugated bilirubin combined). Bilirubin is excreted from the liver into the bile duct and stored in the gallbladder, and is released into the small intestine as bile to aid digestion. When liver function is impaired, bilirubin is not adequately removed from the blood, resulting in elevated bilirubin levels.

The term “ferritin” refers to a hollow globular protein having a molecular weight of 450 kDa and consisting of 24 subunits that functions to store iron in a non-toxic form and transport and release iron to areas where iron is needed. The light type ferritin subunit is encoded by the FTL gene, and the heavy type subunit is encoded by FTH1 gene (also known as FTHL6). Non-limiting examples of ferritin amino acid sequences are set forth in GenBank Accession Nos. NM_000146.3→NP_000137.2 (light chain) and NM_002032.2→NP_002023.2 (heavy chain). Ferritin is stored in many types of cells, including liver cells. When liver cells are damaged, ferritin is released, resulting in elevated serum ferritin levels. Serum ferritin levels greater than 300 ng/mL in men and 200 ng/mL in women are commonly considered to be abnormal.

The term “fibroblast growth factor 21” or “FGF21” refers to a protein that is encoded by the FGF21 gene in mammals. FGF21 is an important metabolic regulator and plays several roles, including controlling AMP activation and insulin sensitivity. FGF21 stimulates glucose uptake in adipocytes, which is an additive effect to that of insulin. In particular, FGF21 levels are increased in patients who have type 2 diabetes. A non-limiting example of an FGF21 amino acid sequence in humans is set forth under GenBank Accession number NM_019113.3→NP_061986.1.

The term “FGFR1c” refers to a cognate receptor of FGF21. In humans, FGFR1c is expressed from the FGFR1 gene. A non-limiting example of an FGFR1c amino acid sequence in humans is set forth under GenBank Accession number NM_001174063.1→NP_001167534.1.

The term “Beta-klotho” refers to a protein encoded by the KLB gene in humans that increases the ability of FGF21 to bind to FGFR1 (e.g., FGFR1c) and FGFR4. In particular, Beta-klotho, FGF21, and the FGF21 receptor must all be present in a binding complex in order for proper activation of the FGF21 receptor by FGF21 to occur. A non-limiting example of a human amino acid sequence is set forth under GenBank Accession number NM_175737.3→NP_783864.1.

### III. Methods for Preventing or Treating Diseases

In one aspect, the present invention provides methods for preventing or treating cancer in a subject (e.g., a subject in need thereof). In some embodiments, the method comprises administering to the subject a therapeutically effective amount of a retinoid and a histone deacetylase (HDAC) inhibitor. In some embodiments, the HDAC inhibitor is a short-chain fatty acid and/or suberanilohydroxamic acid (SAHA). In particular embodiments, the retinoid and/or HDAC inhibitor is a derivative (e.g., enantiomer) thereof or a prodrug thereof (e.g., tributyrin).

As non-limiting examples, retinoids that can be used in methods of the present invention include retinoic acid (RA), retinol, retinal, isotretinoin, alltretinoin, etretinate, acitretin, tazarotene, bexarotene, adapalene, seletinoid G, a retinyl ester, fenretinide, derivatives thereof, and any combination thereof. Suitable retinyl esters include retinyl acetate, retinyl butyrate, retinyl propionate, retinyl palmitate, and any combination thereof. In particular embodiments, the retinoid is RA.

In some embodiments, the HDAC inhibitor that is used is SAHA. In some embodiments, the HDAC inhibitor is an SCFA. Suitable SCFAs include, but are not limited to, formate, acetate, propionate, butyrate, isobutyrate, valerate, isovalerate, and any combination thereof. In particular embodiments, the SCFA is butyrate, propionate, and/or valerate. Any other HDAC inhibitor described herein or known to one of skill in the art can be used.

In some embodiments, a method for preventing or treating cancer in a subject (e.g., a subject in need thereof) comprises administering a microRNA (miR) to the subject. In some embodiments, the miR is miR-22. In some embodiments, the miR is miR-34a. In some embodiments, both miR-22 and miR-34a are administered to the subject. In some embodiments, the miR-22 comprises a nucleotide sequence having at least about 75% identity (e.g., at least about 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to SEQ ID NO:1. In particular embodiments, the miR-22 comprises the nucleotide sequence set forth in SEQ ID NO:1.

In some embodiments, the miR-34a comprises a nucleotide sequence having at least about 75% identity (e.g., at least about 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to SEQ ID NO:2. In particular embodiments, the miR-34a comprises the nucleotide sequence set forth in SEQ ID NO:2.

In some embodiments, a miR mimic is used to prevent or treat cancer in a subject (e.g., a subject in need thereof). Generally, miR mimics are small, chemically modified double-stranded RNA molecules that mimic the effects or activity (e.g., the ability to decrease the expression of a target gene) of an endogenous miR (e.g., miR-22, miR-34a). Generally, miR mimics are designed such that they contain a sequence motif at the 5′ end of the mimic that is at least partially complementary to the target sequence (e.g., in the 3′ untranslated region of a target gene). miR mimics are available commercially, for example from Thermo Fisher or Sigma-Aldrich.

In some embodiments, a miR or mimic thereof used in carrying out the present invention is expressed from a virus. In particular embodiments, a virus containing a nucleotide sequence encoding the miR or mimic thereof is introduced into a cell (e.g., target cell), and then the miR or miR mimic is expressed within the cell. One of ordinary skill in the art will understand that the choice of viral delivery system will depend on the particular indication, target cell type, etc. Non-limiting examples of suitable viruses for expression of miRs and miR mimics include adenovirus, retrovirus, vaccinia virus, poxvirus, adeno-associated virus, herpes simplex virus, and lentivirus. In some embodiments, a polynucleotide comprising a nucleotide sequence encoding the miR or a mimic thereof is introduced into a cell by another method (e.g., lipofection) or as a naked DNA molecule or plasmid.

Methods of the present invention for preventing or treating cancer in a subject (e.g., a subject in need thereof) are suitable for any type of cancer, including but not limited to those described above in Section II. In some embodiments, the subject has liver cancer. In some embodiments, the subject has colon cancer. In some embodiments, the subject has one or more colon polyps.

In some of embodiments, the cancer is an advanced stage cancer (e.g., advanced stage liver or colon cancer). In some embodiments, the cancer is metastatic (e.g., metastatic liver or colon cancer). In some embodiments, treating the subject comprises inhibiting cancer cell growth; inhibiting cancer cell migration; inhibiting cancer cell invasion; ameliorating the symptoms of cancer; reducing the size of a cancer tumor; reducing the number of cancer tumors; reducing the number of cancer cells; inducing cancer cell necrosis, pyroptosis, oncosis, apoptosis, autophagy, or other cell death; or enhancing the therapeutic effects of another anti-cancer agent.

As used herein, the phrase “ameliorating the symptoms of cancer” includes alleviating or improving the symptoms or condition of a patient having cancer (e.g., liver or colon cancer). Ameliorating the symptoms includes reducing the pain or discomfort associated with cancer. Ameliorating the symptoms also includes reducing the markers of cancer, e.g., reducing the number of cancer cells or reducing the size of cancer tumors.

In another aspect, the present invention provides methods for preventing or treating a metabolic disease in a subject (e.g., a subject in need thereof). In some embodiments, the method comprises administering to the subject a therapeutically effective amount of a retinoid and a histone deacetylase (HDAC) inhibitor. In some embodiments, the HDAC inhibitor is a short-chain fatty acid and/or suberanilohydroxamic acid (SAHA). Any of the retinoids and/or HDAC inhibitors described herein can be used to prevent or treat a metabolic disease according to methods of the present invention.

In any of the methods described herein, in some embodiments the method further comprises administering a starch to the subject. In some embodiments, the method further comprises administering a probiotic and/or prebiotic agent to the subject. In some instances, the probiotic agent comprises a bacterium that produces an SCFA. In some instances, the prebiotic comprises apple pectin and/or an inulin.

In any of the methods described herein, in some embodiments, the method further comprises administering a delivery-enhancing agent to the subject. Non-limiting examples of suitable delivery-enhancing agents include cyclodextrins, hepatitis E virus-like particle, an inactivated yeast, an inactivated bacterium, polyvinyl acetate (PVA), an inulin or ester thereof, and a combination thereof. Suitable inulin esters include, but are not limited to, an inulin butyrate ester, an inulin propionate ester, and a combination thereof. In some embodiments, compositions of the present invention are delivered as a nanoemulsion.

When PVA is used, an HDAC inhibitor and retinoid can be packaged into the PVA. In some embodiments, the HDAC inhibitor and the retinoid are packaged in an HDAC inhibitor-retinoid ratio of about 1:10, 1:20, 1:30, 1:40, 1:50, 1:60, 1:70, 1:80, 1:90, 1:100, 1:150, 1:200, 1:250, 1:300, 1:350, 1:400, 1:450, 1:500, 1:550, 1:600, 1:650, 1:700, 1:750, 1:800, 1:850, 1:900, 1:950, or 1:1,000 by weight.

Methods of the present invention are useful for preventing or treating any number of metabolic diseases. In some embodiments, a method or composition of the present invention is used to prevent or treat obesity. In some embodiments, a method or composition of the present invention is used to prevent or treat diabetes (e.g., type 2 diabetes). In particular embodiments, a method or composition of the present invention is used to increase insulin sensitivity. In some embodiments, a method or composition of the present invention is used to prevent or treat NAFLD or NASH. Fatty liver disease (FLD), also known simply as fatty liver or hepatic steatosis, is a condition wherein large vacuoles of triglyceride fat accumulate in hepatocytes via the process of steatosis (i.e., infiltration of liver cells with fat). FLD can occur in individuals who consume little or no alcohol, in which case the disease is known as non-alcoholic fatty liver disease (NAFLD). The accumulation of fat in the liver leads to inflammation and the development of fibrosis within the liver. As the extent of liver fibrosis increases, the development of more severe non-alcoholic steatohepatitis (NASH) occurs. Accompanying the progression of liver fibrosis due to NAFLD and NASH is a progressive deterioration of liver function, possibly leading to liver failure. FLD is estimated to affect about 10 to 20 percent of Americans, with an additional about 2 to 5 percent being affected by the more severe NASH. NASH is often first suspected in an individual who is found to have elevated levels of one or more biomarkers of liver disease (e.g., ALT and AST), particularly when there is no other apparent reason for liver disease (e.g., heavy alcohol intake, medication, or infection such as hepatitis). A suspicion of NASH may also occur when X-ray or other imaging studies show evidence of fatty liver. The gold standard for distinguishing NASH from more benign FLD is to perform a liver biopsy. Suitable biomarkers for the detection and monitoring of liver disease, including NAFLD and NASH, include but are not limited to aspartate aminotransferase (AST), alanine aminotransferase (ALT), the ratio of AST to ALT (i.e., the AST/ALT ratio is often greater than 2 in progressive NASH), gamma-glutamyl transferase (GGT), the aspartate to platelet ratio index (APRI), alkaline phosphatase (AP), bilirubin, and ferritin.

In particular embodiments, a test sample is obtained from the subject. The test sample can be obtained before and/or after the retinoid and HDAC inhibitor, the miR or mimic thereof, or pharmaceutical composition is administered to the subject. Non-limiting examples of suitable samples include blood, serum, plasma, cerebrospinal fluid, tissue, saliva, urine or any combination thereof. In some instances, the sample comprises normal tissue. In other instances, the sample comprises cancer tissue. The sample can also be made up of normal and/or cancer cells. Tissue samples can be obtained by biopsy or surgical resection.

In some embodiments, a reference sample is obtained. The reference sample can be obtained, for example, from the subject and can comprise normal tissue. The reference sample can be also be obtained from a different subject and/or a population of subjects. In some instances, the reference sample is either obtained from the subject, a different subject, or a population of subjects before and/or after the retinoid and HDAC inhibitor, the miR or mimic thereof, or pharmaceutical composition is administered to the subject, and comprises normal tissue. However, in some instances the reference sample comprises cancer tissue and is obtained from the subject and/or from a different subject or a population of subjects.

In some embodiments, the level of one or more biomarkers is determined in the test sample and/or reference sample. Non-limiting examples of suitable biomarkers include miR such as miR-22 and miR-34a. In some embodiments, at least one of the biomarkers is a miR. Other non-limiting examples of suitable biomarkers include FGF21, FGFR1c, Beta-klotho, blood glucose, aspartate aminotransferase (AST), alanine aminotransferase (ALT), the ratio of AST to ALT, gamma-glutamyl transferase (GGT), the aspartate to platelet ratio index (APRI), alkaline phosphatase (AP), bilirubin, ferritin, alpha-smooth muscle actin (αSMA), procollagen α1 (procol1), transforming growth factor-β (TGFβ), monocyte chemoattractant protein-1 (MCP1), interleukin-1β (IL-1b), tumor necrosis factor alpha (TNFα), connective tissue growth factor (CTGF), and platelet derived growth factor receptor beta (PDGFRβ). Any combination of biomarkers, including those described herein and others that will readily be known to one of skill in the art, can be used.

Typically, the level of the one or more biomarkers in one or more test samples is compared to the level of the one or more biomarkers in one or more reference samples. As a non-limiting example, levels of one or biomarkers in test samples taken before and after the retinoid and HDAC inhibitor, the miR or mimic thereof, or pharmaceutical composition is administered to the subject are compared to the level of the one or more biomarkers in a reference sample that is either normal tissue obtained from the subject, or normal tissue that is obtained from a different subject or a population of subjects. In some instances, the biomarker in a test sample obtained from the subject before the subject is treated is lower than the level of the biomarker in the reference sample. In other instances, the level of biomarker in a test sample obtained from the subject after the subject is treated is increased relative to the level of the biomarker in a test sample obtained prior to administration.

The differences between the reference sample or value and the test sample need only be sufficient to be detected. In some embodiments, a decreased level of a biomarker in the test sample, and hence the presence of cancer or increased risk of cancer, or the presence of a metabolic disease or the risk of a metabolic disease, is determined when the biomarker levels are at least, e.g., 10%, 25%, 50% or more lower in comparison to a negative control. In some embodiments, an increased level of a biomarker in the test sample, and hence the presence of cancer or increased risk of cancer, or the presence of a metabolic disease or the risk of a metabolic disease, is determined when the biomarker levels are at least, e.g., 10%, 25%, 50% or more greater in comparison to a negative control.

The biomarker levels can be detected using any method known in the art, including the use of antibodies specific for the biomarkers. Exemplary methods include, without limitation, Western Blot, dot blot, enzyme-linked immunosorbent assay (ELISA), radioimmunoassay (RIA), immunoprecipitation, immunofluorescence, FACS analysis, electrochemiluminescence, and multiplex bead assays (e.g., using Luminex or fluorescent microbeads).

In some embodiments, the antibody or plurality thereof used to detect the biomarker(s) can be immobilized on a solid support. The solid support can be, for example, a multiwell plate, a microarray, a chip, a bead, a porous strip, or a nitrocellulose filter. In some instances, the bead comprises chitin. The immobilization can be via covalent or non-covalent binding.

Labeled secondary antibodies can be used to detect binding between antibodies and biomarkers. Secondary antibodies bind to the constant or “C” regions of different classes or isotypes of immunoglobulins IgM, IgD, IgG, IgA, and IgE. Usually, a secondary antibody against an IgG constant region is used in the present methods. Secondary antibodies against the IgG subclasses, for example, IgG1, IgG2, IgG3, and IgG4, also find use in the present methods. Secondary antibodies can be labeled with any directly or indirectly detectable moiety, including a fluorophore (e.g., fluorescein, phycoerythrin, quantum dot, Luminex bead, fluorescent bead), an enzyme (e.g., peroxidase, alkaline phosphatase), a radioisotope (e.g., ³H, ³²P, ¹²⁵I) or a chemiluminescent moiety. Labeling signals can be amplified using a complex of biotin and a biotin binding moiety (e.g., avidin, streptavidin, neutravidin). Fluorescently labeled anti-human IgG antibodies are commercially available from Molecular Probes, Eugene, Oreg. Enzyme-labeled anti-human IgG antibodies are commercially available from Sigma-Aldrich, St. Louis, Mo. and Chemicon, Temecula, Calif.

General immunoassay techniques are well known in the art. Guidance for optimization of parameters can be found in, for example, Wu, Quantitative Immunoassay: A Practical Guide for Assay Establishment, Troubleshooting, and Clinical Application, 2000, AACC Press; Principles and Practice of Immunoassay, Price and Newman, eds., 1997, Groves Dictionaries, Inc.; The Immunoassay Handbook, Wild, ed., 2005, Elsevier Science Ltd.; Ghindilis, Pavlov and Atanassov, Immunoassay Methods and Protocols, 2003, Humana Press; Harlow and Lane, Using Antibodies: A Laboratory Manual, 1998, Cold Spring Harbor Laboratory Press; and Immunoassay Automation: An Updated Guide to Systems, Chan, ed., 1996, Academic Press.

In certain embodiments, the presence or decreased or increased presence of one or more biomarkers is indicated by a detectable signal (e.g., a blot, fluorescence, chemiluminescence, color, radioactivity) in an immunoassay. This detectable signal can be compared to the signal from a control sample or to a threshold value. In some embodiments, decreased presence is detected, and the presence or increased risk of cancer is indicated, when the detectable signal of biomarker(s) in the test sample is at least about 10%, 20%, 30%, 50%, 75% lower in comparison to the signal of antibodies in the reference sample or the predetermined threshold value. In other embodiments, an increased presence is detected, and the presence or increased risk of cancer is indicated, when the detectable signal of biomarker(s) in the test sample is at least about 1-fold, 2-fold, 3-fold, 4-fold or more, greater in comparison to the signal of antibodies in the reference sample or the predetermined threshold value.

In some embodiments, the results of the biomarker level determinations are recorded in a tangible medium. For example, the results of diagnostic assays (e.g., the observation of the presence or decreased or increased presence of one or more biomarkers) and the diagnosis of whether or not there is an increased risk or the presence of cancer or a metabolic disease can be recorded, e.g., on paper or on electronic media (e.g., audio tape, a computer disk, a CD, a flash drive, etc.).

In other embodiments, the methods further comprise the step of providing the diagnosis to the patient (i.e., the subject) and/or the results of treatment.

### IV. Compositions and Administration

In another aspect, the present invention provides pharmaceutical compositions. In some embodiments, the pharmaceutical composition comprises a retinoid, a histone deacetylase (HDAC) inhibitor, and a pharmaceutically acceptable carrier. In some embodiments, the HDAC inhibitor is a short-chain fatty acid and/or suberanilohydroxamic acid (SAHA). In particular embodiments, the retinoid and/or HDAC inhibitor is a derivative (e.g., enantiomer) thereof or a prodrug thereof (e.g., tributyrin).

As non-limiting examples, retinoids that can be used in compositions of the present invention include retinoic acid (RA), retinol, retinal, isotretinoin, alltretinoin, etretinate, acitretin, tazarotene, bexarotene, adapalene, seletinoid G, a retinyl ester, fenretinide, derivatives thereof, and any combination thereof. Suitable retinyl esters include retinyl acetate, retinyl butyrate, retinyl propionate, retinyl palmitate, and any combination thereof. In particular embodiments, the retinoid is RA.

In some embodiments, the concentration of the retinoid (e.g., RA) is about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 μM, or more. In particular embodiments, the concentration of the retinoid (e.g., RA) is about 10 μM.

In some embodiments, the HDAC inhibitor that is used is SAHA. In some embodiments, the HDAC inhibitor is an SCFA. Suitable SCFAs include, but are not limited to, formate, acetate, propionate, butyrate, isobutyrate, valerate, isovalerate, and any combination thereof. In particular embodiments, the SCFA is butyrate, propionate, and/or valerate. Any other HDAC inhibitor described herein or known to one of skill in the art can be used.

In some embodiments, the concentration of SAHA is about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 μM, or more. In particular embodiments, the concentration of SAHA is about 5 μM.

In some embodiments, the concentration of the SCFA is about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 mM, or more. In particular embodiments, the concentration is about 5 mM. In some embodiments, the concentration of butyrate, propionate, and/or valerate is about 5 mM. In some embodiments, the concentration of butyrate, propionate, and/or valerate is about 10 mM.

In some embodiments, a pharmaceutical composition of the present invention comprises a microRNA (miR) or a mimic thereof and a pharmaceutically acceptable carrier. In some embodiments, the pharmaceutical composition comprises miR-22. In some embodiments, the pharmaceutical composition comprises miR-34a. In some embodiments, the pharmaceutical composition comprises both miR-22 and miR-34a. In some embodiments, the miR-22 comprises a nucleotide sequence having at least about 75% identity (e.g., at least about 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to SEQ ID NO:1. In particular embodiments, the miR-22 comprises the nucleotide sequence set forth in SEQ ID NO:1.

In some embodiments, the miR-34a comprises a nucleotide sequence having at least about 75% identity (e.g., at least about 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to SEQ ID NO:2. In particular embodiments, the miR-34a comprises the nucleotide sequence set forth in SEQ ID NO:2.

Pharmaceutical compositions or medicaments for use in the present invention can be formulated by standard techniques using one or more physiologically acceptable carriers or excipients. Suitable pharmaceutical carriers are described herein and in “Remington's Pharmaceutical Sciences” by E. W. Martin. Compounds and agents of the present invention and their physiologically acceptable salts and solvates can be formulated for administration by any suitable route, including via inhalation, topically, nasally, orally, intravenously, parenterally, or rectally.

In some embodiments, a pharmaceutical composition described herein comprises a nanoemulsion. In some embodiments, a pharmaceutical composition of the present invention further comprises a starch. In other embodiments, the pharmaceutical composition further comprises a probiotic agent. In some other embodiments, the pharmaceutical composition comprises a prebiotic agent. In some embodiments, the pharmaceutical composition further comprises both a probiotic agent and a prebiotic agent. Suitable prebiotic agents include, but are not limited to, apple pectin, inulin (or an ester thereof), and a combination thereof. In some instances, a probiotic agent is a bacterium that produces an SCFA (e.g., butyrate, propionate) such as Roseburia hominis or Propionibacterium freudenreichii.

In some embodiments, a pharmaceutical composition of the present invention further comprises a delivery-enhancing agent. In some embodiments, the delivery-enhancing agent comprises a cyclodextrin. Cyclodextrins, which are a family of compounds that comprise cyclic oligosaccharides, can take the form of alpha-cyclodextrins (having a 6-membered ring), beta-cyclodextrins (having a 7-membered ring), or gamma cyclodextrins (having an 8-membered ring). Cyclodextrins can increase the aqueous solubility of compounds and can increase bioavailability and stability. Folate-conjugated amphiphilic cyclodextrins and derivatives thereof can be used for tumor targeting. Polycationic amphiphilic cyclodextrins enhance the interaction of compounds with cell membranes. Non-limiting examples of particularly useful cyclodextrins include Captisol® and Dexolve™ (sulfobutyl-ether-beta-cyclodextrin). Captisol® is useful for, among other things, improving the solubility, stability, bioavailability or compounds for administration, as well as decreasing volatility, irritation, smell, or taste.

In some embodiments, a delivery-enhancing agent comprises a hepatitis E virus-like particle (HEV-VLP). HEV-VLP can serve as both an immunogen and a non-infectious drug delivery agent. HEV-VLP is particularly useful in the setting of cancer, where the agent can target compounds of interest to cancer tissue. HEV-VLP is further described in U.S. Pat. Nos. 8,906,862, 8,906,863, and 9,637,524 and U.S. Patent Application Publication No. US 2017/0107261, hereby incorporated by reference for all purposes.

In some embodiments, a delivery-enhancing agent comprises inactivated bacteria or yeast. Encapsulating retinoids and/or HDAC inhibitors described herein, or miRs described herein, into inactivated bacteria and yeast is especially useful for oral administration, as the retinoids, HDAC inhibitors, and miRs can be delivered to the gut with increased inactivity. This method is further described in PCT Application Publication No. WO/2016/069740, hereby incorporated by reference for all purposes.

As a non-limiting example, heat-inactivated yeast cells can be suspended in 35% ethanol that contains an SCFA (e.g., butyrate or propionate) and sealed in 6×8 boilable vacuum bags (Prime Source). The sample is then subjected to pressure. Subsequently, the samples are pelleted and washed (e.g., using 35% ethanol, followed by water) to remove un-encapsulated chemicals. The encapsulation efficiency can be quantified by sonication and extraction using 100% methanol followed by gas chromatography to determine the recovery rate.

In some embodiments, the delivery-enhancing agent comprises polyvinyl acetate (PVA), which is a synthetic resin having the formula (C₄H₆O₂)ₙ and is formed by the polymerization of vinyl acetate. PVA allows two or more compounds, including those having differences in solubility (e.g., aqueous solubility) to be packaged together (e.g., in one tablet). As a non-limiting example, an HDAC inhibitor and retinoid can be packaged into the PVA. In some instances, a retinoid (e.g., retinoic acid) and propionate are packaged together in the PVA. In some embodiments, a retinoid and butyrate are packaged together in the PVA. In some embodiments, a retinoid and valerate are packaged together in the PVA.

In some embodiments, the HDAC inhibitor and the retinoid are packaged in an HDAC inhibitor-retinoid ratio of about 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:150, 1:200, 1:250, 1:300, 1:350, 1:400, 1:450, 1:500, 1:550, 1:600, 1:650, 1:700, 1:750, 1:800, 1:850, 1:900, 1:950, or 1:1,000 by weight.

In some embodiments, the HDAC inhibitor and the retinoid are packaged in an HDAC inhibitor-retinoid ratio between about 1:10 and about 1:100, about 1:50 and about 1:100, about 1:20 and about 1:100, about 1:50 and about 1:200, about 1:100 and about 1:200, about 1:100 and about 1:300, about 1:100 and about 1:400, about 1:100 and about 1:500, about 1:100 and about 1:600, about 1:100 and about 1:700, about 1:100 and about 1:800, about 1:100 and about 1:900, or about 1:500 and about 1:1,000 by weight.

Furthermore, for compounds that do not have carboxyl group (e.g., fenretinide), a linker (e.g., succinic acid) can be conjugated to the compound to enable packaging of the compound into PVA. will be introduced to package them into PVA. PVA is further described in PCT Application Publication No. WO/2017/031084, hereby incorporated by reference for all purposes.

In some embodiments, the delivery-enhancing agent comprises an inulin. Inulins are a class of naturally occurring polysaccharides that belong to a class of dietary fibers known as fructans. In humans, inulins are indigestible, whereas bacterial fermentation can lead to the generation of butyrate and propionate from inulins. Because of their resistance to acids and human digestive enzymes, inulins find utility for oral drug delivery, in particular the delivery of drugs to the colon, where they can be readily absorbed through the gut epithelium. Inulin esters are also useful for methods and compositions of the present invention. Suitable inulin esters include, but are not limited to inulin butyrate esters, inulin propionate esters, and a combination thereof.

In some embodiments, an active agent (e.g., a retinoid, HDAC inhibitor, or a combination thereof, or a miR) in encapsulated (e.g., nanoencapsulated). In some embodiments, the compositions of the present invention comprise active agents that are encapsulated (e.g., with glucosamine butyrate or a glucosamine butyrate-gelatin matrix). In some embodiments, an active agent is encapsulated in a matrix that comprises an emulsifier (e.g., a monoester, diester, or organic ester of a glyceride), a carbohydrate hydrocolloid, an unmodified or modified starch, a pectin, a giucan, a cyclodextrin, a maltodextrin, or a protein (e.g., a casein, whey, soy).

Furthermore, one or more active agents (e.g., a retinoid, an HDAC inhibitor, or a miR) can be complexed, e.g., in a liposome, in a nanoparticle, in a supramolecular assembly, or an ion pair.

In some embodiments, a composition of the present invention comprises a Eudragit® polymer. Eudragit® is useful for protecting compounds from being dissolved in the stomach, allowing them to be available for release and in more distal regions of the GI tract. Eudragit® L, S, FS, and E polymers are available with acidic or alkaline groups that allow for pH-dependent drug release. Eudragit® RL and RS polymers (cationic groups) and Eudragit® NM polymer with neutral groups enable time-release of drugs. Eudragit® 15 commercially available from Evonik.

In some embodiments, targeting delivery of an active agent or compound (e.g., delivery of a retinoid and an HDAC inhibitor, or delivery of a miR) to the colon is especially desired. While useful for other routes and modes of delivery, encapsulation of active agents or compounds in polymeric micelles, inulins (and esters thereof), nanoparticles, or cross-linked chitosan microspheres are especially useful for delivery to the colon.

For inulin-based delivery (e.g., tablets and capsules), a three-component design can be used, wherein the three components include: (1) a hard gelatin enteric-coated capsule (for carrying two pulses), (2) first-pulse granules (for rapid release in intestine), and (3) second-pulse matrix tablet (for slow release in the colon).

Nanoparticles can be made with Eudragit® S100. Alternatively, mucoadhesive nanoparticles can be created with trimethylchitosan (TMC). Also, a mix of polymers (e.g., PLGA, PEG-PLGA, and PEG-PCL) can be used to obtain a sustained drug delivery.

For cross-linked chitosan microspheres, a multiparticulate system comprising pH-sensitive properties and specific biodegradability for colon-targeted delivery of agents such as retinoids, HDAC inhibitors, and miRs can be used. As a non-limiting example, cross-linked chitosan microspheres can be prepared from an emulsion system using liquid paraffin as the external phase and a solution of chitosan in acetic acid as the disperse phase. The multiparticulate system is prepared by coating cross-linked chitosan microspheres exploiting Eudragit® L-100 and S-100 as pH-sensitive polymers.

Furthermore, cellulose acetate butyrate (CAB) can be used to enhance colonic delivery (e.g., of a retinoid and an HDAC inhibitor, or a miR).

In some embodiments, a composition of the present invention comprises an active agent (e.g., a retinoid, HDAC inhibitor, or miR) in an amount that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% by weight. In some embodiments, the active agent is between about 1%-10%, 1%-20%, 1%-30%, 1%-40%, 1%-50%, 1%-60%, 1%-70%, 1%-80%, 1%-90%, 1%-100%, 10%-20%, 10%-30%, 10%-40%, 10%-50%, 10%-60%, 10%-70, 10%-80, 10%-90, 10%-100%, 20%-30%, 20%-40%, 20%-50%, 20%-60%, 20%-70%, 20%-80%, 20%-90%, 20%-100%, 30%-40%, 30%-50%, 30%-60%, 30%-70%, 30%-80%, 30%-90%, 30%-100%, 40%-50%, 40%-60%, 40%-70%, 40%-80%, 40%-90%, 40%-100%, 50%-60%, 50%-70%, 50%-80%, 50%-90%, 50%-100%, 60%-70%, 60%-80%, 60%-90%, 60%-100%, 70%-80%, 70%-90%, 70%-100%, 80%-90%, 80%-100%, or 90%-100% by weight.

In some embodiments, a composition of the present invention comprises an active agent (e.g., a retinoid, HDAC inhibitor, or miR) in an amount that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% by volume. In some embodiments, the active agent is between about 1%-10%, 1%-20%, 1%-30%, 1%-40%, 1%-50%, 1%-60%, 1%-70%, 1%-80%, 1%-90%, 1%-100%, 10%-20%, 10%-30%, 10%-40%, 10%-50%, 10%-60%, 10%-70, 10%-80, 10%-90, 10%-100%, 20%-30%, 20%-40%, 20%-50%, 20%-60%, 20%-70%, 20%-80%, 20%-90%, 20%-100%, 30%-40%, 30%-50%, 30%-60%, 30%-70%, 30%-80%, 30%-90%, 30%-100%, 40%-50%, 40%-60%, 40%-70%, 40%-80%, 40%-90%, 40%-100%, 50%-60%, 50%-70%, 50%-80%, 50%-90%, 50%-100%, 60%-70%, 60%-80%, 60%-90%, 60%-100%, 70%-80%, 70%-90%, 70%-100%, 80%-90%, 80%-100%, or 90%400% by volume.

In some embodiments, a composition of the present invention comprises an inactive agent (i.e., not a retinoid, HDAC inhibitor, or miR) in an amount that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% by weight. In some embodiments, the inactive agent is between about 1%-10%, 1%-20%, 1%-30%, 1%-40%, 1%-50%, 1%-60%, 1%-70%, 1%-80%, 1%-90%, 1%-100%, 10%-20%, 10%-30%, 10%-40%, 10%-50%, 10%-60%, 10%-70, 10%-80, 10%-90, 10%-100%, 20%-30%, 20%-40%, 20%-50%, 20%-60%, 20%-70%, 20%-80%, 20%-90%, 20%-100%, 30%-40%, 30%-50%, 30%-60%, 30%-70%, 30%-80%, 30%-90%, 30%-100%, 40%-50%, 40%-60%, 40%-70%, 40%-80%, 40%-90%, 40%-100%, 50%-60%, 50%-70%, 50%-80%, 50%-90%, 50%-100%, 60%-70%, 60%-80%, 60%-90%, 60%-100%, 70%-80%, 70%-90%, 70%-100%, 80%-90%, 80%-100%, or 90%400% by weight.

In some embodiments, a composition of the present invention comprises an inactive agent (i.e., an agent or compound present in the composition that is not a retinoid, HDAC inhibitor, or miR) in an amount that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% by volume. In some embodiments, the inactive agent is between about 1%-10%, 1%-20%, 1%-30%, 1%-40%, 1%-50%, 1%-60%, 1%-70%, 1%-80%, 1%-90%, 1%-100%, 10%-20%, 10%-30%, 10%-40%, 10%-50%, 10%-60%, 10%-70, 10%-80, 10%-90, 10%-100%, 20%-30%, 20%-40%, 20%-50%, 20%-60%, 20%-70%, 20%-80%, 20%-90%, 20%-100%, 30%-40%, 30%-50%, 30%-60%, 30%-70%, 30%-80%, 30%-90%, 30%-100%, 40%-50%, 40%-60%, 40%-70%, 40%-80%, 40%-90%, 40%-100%, 50%-60%, 50%-70%, 50%-80%, 50%-90%, 50%-100%, 60%-70%, 60%-80%, 60%-90%, 60%-100%, 70%-80%, 70%-90%, 70%-100%, 80%-90%, 80%-100%, or 90%400% by volume.

In pharmaceutical compositions that comprise a delivery enhancing agent, in some embodiments, the delivery-enhancing agent is present in an amount that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% by weight. In some embodiments, the delivery-enhancing agent is between about 1%-10%, 1%-20%, 1%-30%, 1%-40%, 1%-50%, 1%-60%, 1%-70%, 1%-80%, 1%-90%, 1%-100%, 10%-20%, 10%-30%, 10%-40%, 10%-50%, 10%-60%, 10%-70, 10%-80, 10%-90, 10%-100%, 20%-30%, 20%-40%, 20%-50%, 20%-60%, 20%-70%, 20%-80%, 20%-90%, 20%-100%, 30%-40%, 30%-50%, 30%-60%, 30%-70%, 30%-80%, 30%-90%, 30%-100%, 40%-50%, 40%-60%, 40%-70%, 40%-80%, 40%-90%, 40%400%, 50%-60%, 50%-70%, 50%-80%, 50%-90%, 50%-100%, 60%-70%, 60%-80%, 60%-90%, 60%-100%, 70%-80%, 70%-90%, 70%-100%, 80%-90%, 80%-100%, or 90%400% by weight.

In some embodiments, the delivery-enhancing agent is present in an amount that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% by volume. In some embodiments, the delivery-enhancing agent is between about 1%-10%, 1%-20%, 1%-30%, 1%-40%, 1%-50%, 1%-60%, 1%-70%, 1%-80%, 1%-90%, 1%-100%, 10%-20%, 10%-30%, 10%-40%, 10%-50%, 10%-60%, 10%-70, 10%-80, 10%-90, 10%-100%, 20%-30%, 20%-40%, 20%-50%, 20%-60%, 20%-70%, 20%-80%, 20%-90%, 20%-100%, 30%-40%, 30%-50%, 30%-60%, 30%-70%, 30%-80%, 30%-90%, 30%-100%, 40%-50%, 40%-60%, 40%-70%, 40%-80%, 40%-90%, 40%-100%, 50%-60%, 50%-70%, 50%-80%, 50%-90%, 50%-100%, 60%-70%, 60%-80%, 60%-90%, 60%-100%, 70%-80%, 70%-90%, 70%-100%, 80%-90%, 80%-100%, or 90%400% by volume

a. Routes of Administration

Typical formulations for topical administration include creams, ointments, sprays, lotions, and patches. The pharmaceutical composition can, however, be formulated for any type of administration, e.g., intradermal, subdermal, intravenous, intramuscular, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral or rectal administration is also contemplated.

Suitable formulations for transdermal application include an effective amount of one or more compositions or compounds described herein, optionally with a carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

For oral administration, a pharmaceutical formulation or a medicament can take the form of, for example, a tablet or a capsule prepared by conventional means with a pharmaceutically acceptable excipient. The present invention provides tablets and gelatin capsules comprising: (1) a HDAC inhibitor and/or a retinoid, alone or in combination with other compounds, or a dried solid powder of these drugs, or (2) a miR, alone or in combination with other compounds, or a dried solid powder of these compounds, together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, talcum, stearic acid, magnesium or calcium salt, metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, and/or (0 absorbents, colorants, flavors and sweeteners. In some embodiments, an amorphous solid dispersion of an active agent (e.g., an HDAC inhibitor, retinoid, or analog thereof, or a miR) is prepared that is suitable for oral delivery.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound(s).

The compositions and formulations set forth herein can be formulated for parenteral administration by injection, for example by bolus injection or continuous infusion. Formulations for injection can be presented in unit dosage form, for example, in ampules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient(s) can be in powder form for constitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the active ingredient(s).

For administration by inhalation, the compositions of the present invention may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

The compositions set forth herein can also be formulated in rectal compositions, for example, suppositories or retention enemas, for example, containing conventional suppository bases, for example, cocoa butter or other glycerides.

Furthermore, the active ingredient(s) can be formulated as a depot preparation. Such long-acting formulations can be administered by implantation (for example, subcutaneously or intramuscularly) or by intramuscular injection. Thus, for example, one or more of the compounds described herein can be formulated with suitable polymeric or hydrophobic materials (for example as an emulsion in an acceptable oil) or ion exchange resins, or as sparingly soluble derivatives, for example, as a sparingly soluble salt.

In particular embodiments, a pharmaceutical composition or medicament of the present invention can comprise (i) a therapeutically effective amount of a retinoid (e.g., retinoic acid), and/or (ii) a therapeutically effective amount of an HDAC inhibitor (e.g., SAHA or an SCFA such as butyrate, propionate, or valerate), alone or in combination with other compounds. In other embodiments, a pharmaceutical composition or medicament of the present invention can comprise a therapeutically effective amount of a miR (e.g., miR-22 or miR-34a) or a mimic thereof, alone or in combination with other compounds. The therapeutic agent(s) may be used individually, sequentially, or in combination with one or more other such therapeutic agents (e.g., a first therapeutic agent, a second therapeutic agent, a compound of the present invention, etc.). Administration may be by the same or different route of administration or together in the same pharmaceutical formulation.

b. Dosage

Pharmaceutical compositions or medicaments can be administered to a subject at a therapeutically effective dose to prevent, treat, re-sensitize, or control cancer (e.g., liver or colon cancer), or prevent, treat, or control a metabolic disease (e.g., NASH, NAFLD, diabetes, or obesity), as described herein. The pharmaceutical composition or medicament is administered to a subject in an amount sufficient to elicit an effective therapeutic response in the subject.

The dosage of active agents administered is dependent on the subject's body weight, age, individual condition, surface area or volume of the area to be treated and on the form of administration. The size of the dose also will be determined by the existence, nature, and extent of any adverse effects that accompany the administration of a particular formulation in a particular subject. A unit dosage for oral administration to a mammal of about 50 to about 70 kg may contain between about 5 and about 500 mg, about 25-200 mg, about 100 and about 1000 mg, about 200 and about 2000 mg, about 500 and about 5000 mg, or between about 1000 and about 2000 mg of the active ingredient. A unit dosage for oral administration to a mammal of about 50 to about 70 kg may contain about 10 mg, 20 mg, 25 mg, 50 mg, 75 mg, 100 mg, 200 mg, 300 mg, 400 mg, 500 mg, 600 mg, 700 mg, 800 mg, 900 mg, 1,000 mg, 1,250 mg, 1,500 mg, 2,000 mg, 2,500 mg, 3,000 mg, or more of the active ingredient. Typically, a dosage of the active compound(s) of the present invention is a dosage that is sufficient to achieve the desired effect. Optimal dosing schedules can be calculated from measurements of active agent accumulation in the body of a subject. In general, dosage may be given once or more of daily, weekly, or monthly. Persons of ordinary skill in the art can easily determine optimum dosages, dosing methodologies and repetition rates.

Optimum dosages, toxicity, and therapeutic efficacy of the compositions of the present invention may vary depending on the relative potency of the administered composition and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Agents that exhibit large therapeutic indices are preferred. While agents that exhibit toxic side effects can be used, care should be taken to design a delivery system that targets such agents to the site of affected tissue to minimize potential damage to normal cells and, thereby, reduce side effects.

Optimal dosing schedules can be calculated from measurements of active ingredient accumulation in the body of a subject. In general, dosage is from about 1 ng to about 1,000 mg per kg of body weight and may be given once or more daily, weekly, monthly, or yearly. Persons of ordinary skill in the art can easily determine optimum dosages, dosing methodologies and repetition rates. One of skill in the art will be able to determine optimal dosing for administration of retinoids (e.g., retinoic acid), HDAC inhibitors (e.g., SAHA and SCFAs such as butyrate, propionate, and valerate), and miRs, to a human being following established protocols known in the art and the disclosure herein.

The data obtained from, for example, animal studies (e.g. rodents and monkeys) can be used to formulate a dosage range for use in humans. The dosage of compounds of the present invention lies preferably within a range of circulating concentrations that include the ED₅₀ with little or no toxicity. The dosage can vary within this range depending upon the dosage form employed and the route of administration. For any composition for use in the methods of the invention, the therapeutically effective dose can be estimated initially from cell culture assays. A dose can be formulated in animal models to achieve a circulating plasma concentration range that includes the ICso (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of a chimeric protein, preferably a composition is from about 1 ng/kg to about 100 mg/kg for a typical subject.

A typical composition of the present invention for oral or intravenous administration can be about 0.1 to about 10 mg of active ingredient per patient per day; about 1 to about 100 mg per patient per day; about 25 to about 200 mg per patient per day; about 50 to about 500 mg per patient per day; about 100 to about 1000 mg per patient per day; or about 1000 to about 2000 mg per patient per day. Exemplary dosages include, but are not limited to, about 10 mg, 20 mg, 25 mg, 50 mg, 75 mg, 100 mg, 200 mg, 300 mg, 400 mg, 500 mg, 600 mg, 700 mg, 800 mg, 900 mg, 1,000 mg, 1,250 mg, 1,500 mg, 2,000 mg, 2,500 mg, 3,000 mg, or more of the active ingredient per patient per day. Methods for preparing administrable compositions will be known or apparent to those skilled in the art and are described in more detail in such publications as Remington: The Science and Practice of Pharmacy, 21^{st} Ed., University of the Sciences in Philadelphia, Lippencott Williams & Wilkins (2005).

Exemplary doses of the compositions described herein include milligram or microgram amounts of the composition per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a composition depend upon the potency of the composition with respect to the desired effect to be achieved. When one or more of these compositions is to be administered to a mammal, a physician, veterinarian, or researcher may, for example, prescribe a relatively low dose at first, subsequently increasing the dose until an appropriate response is obtained. In addition, it is understood that the specific dose level for any particular mammal subject will depend upon a variety of factors including the activity of the specific composition employed, the age, body weight, general health, gender, and diet of the subject, the time of administration, the route of administration, the rate of excretion, any drug combination, and the degree of expression or activity to be modulated.

In some embodiments, a pharmaceutical composition or medicament of the present invention is administered, e.g., in a daily dose in the range from about 1 mg of compound per kg of subject weight (1 mg/kg) to about 1 g/kg. In another embodiment, the dose is a dose in the range of about 5 mg/kg to about 500 mg/kg. In yet another embodiment, the dose is about 10 mg/kg to about 250 mg/kg. In another embodiment, the dose is about 25 mg/kg to about 150 mg/kg. A preferred dose is about 2, 3, 4, 5, 6, 7, 8, 9, 10, 12, 15, 18, 20, 25, 30, 40, or 50 mg/kg. The daily dose can be administered once per day or divided into subdoses and administered in multiple doses, e.g., twice, three times, or four times per day. However, as will be appreciated by a skilled artisan, compositions described herein may be administered in different amounts and at different times. The skilled artisan will also appreciate that certain factors may influence the dosage and timing required to effectively treat a subject, including but not limited to the severity of the disease or malignant condition, previous treatments, the general health and/or age of the subject, and other diseases present. Moreover, treatment of a subject with a therapeutically effective amount of a composition can include a single treatment or, preferably, can include a series of treatments.

To achieve the desired therapeutic effect, compounds or agents described herein may be administered for multiple days at the therapeutically effective daily dose. Thus, therapeutically effective administration of compounds to treat cancer (e.g., liver or colon cancer) or a metabolic disease (e.g., obesity, diabetes, NASH, or NAFLD) in a subject may require periodic (e.g., daily) administration that continues for a period ranging from three days to two weeks or longer. Compositions set forth herein may be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the agents are not administered daily, so long as the administration is repeated frequently enough to maintain a therapeutically effective concentration of the agents in the subject. For example, one can administer the agents every other day, every third day, or, if higher dose ranges are employed and tolerated by the subject, once a week, once every two weeks, once every three weeks, once every four weeks, or even less frequently.

In some cases, the recitation of a dose “per day” refers to the amount of drug administered each day. In other cases, the “per day” dose refers to the average amount per day of drug administered over a period of time. Thus, if a drug is administered once a week at 100 mg, then the “per day” dose would be approximately equal to (100 mg/7 days=) 14.3 mg per day.

Following successful treatment, it may be desirable to have the subject undergo maintenance therapy to prevent the recurrence of the cancer (e.g., liver or colon cancer) or metabolic disease (e.g., obesity, diabetes, NASH, or NAFLD).

Determination of an effective amount is well within the capability of those skilled in the art, especially in light of the detailed disclosure provided herein. Generally, an efficacious or effective amount of an composition is determined by first administering a low dose or small amount of the composition, and then incrementally increasing the administered dose or dosages, adding a second or third medication as needed, until a desired effect of is observed in the treated subject with minimal or no toxic side effects.

Single or multiple administrations of the compositions are administered depending on the dosage and frequency as required and tolerated by the patient. In any event, the composition should provide a sufficient quantity of the compositions of this invention to effectively treat the patient. Generally, the dose is sufficient to treat, improve, or ameliorate symptoms or signs of disease without producing unacceptable toxicity to the patient.

### V. Kits, Containers, Devices, and Systems

A wide variety of kits, systems, and compositions can be prepared according to the present invention, depending upon the intended user of the kit and system and the particular needs of the user. In some embodiments, the present invention provides a kit that includes a retinoid (e.g., retinoic acid (RA)) and/or an HDAC inhibitor (e.g., a short-chain fatty acid (SCFA) or suberanilohydroxamic acid (SAHA)), alone or in combination with other compounds. In some embodiments, the kit comprises a microRNA (miR), e.g., miR-22 or miR-34a. In some embodiments, the kit contains a pharmaceutical composition of the present invention as described herein.

In some embodiments, the present invention provides a kit that includes a container containing a retinoid (e.g., retinoic acid) and a container (e.g., a separate container) containing an HDAC inhibitor (e.g., a short-chain fatty acid (SCFA) or suberanilohydroxamic acid (SAHA)). In some embodiments, the kit includes a container containing a miR (e.g., miR-22 or miR-34a).

The retinoid in the kit can be any suitable retinoid including, but not limited to, retinoic acid (RA), retinol, retinal, isotretinoin, alltretinoin, etretinate, acitretin, tazarotene, bexarotene, adapalene, seletinoid G, a retinyl ester, fenretinide, derivatives thereof, or a combination thereof. The retinyl ester can be, for example, retinyl acetate, retinyl butyrate, retinyl propionate, retinyl palmitate, or a combination thereof.

The HDAC inhibitor in the kit can be any suitable HDAC inhibitor including, but not limited to, a short-chain fatty acid (SCFA), suberanilohydroxamic acid (SAHA), or a combination thereof. The SCFA can be, for example, butyrate, propionate, valerate, or a combination thereof.

The compositions of the present invention, including but not limited to compositions containing a miR or compositions comprising a retinoid, such as retinoic acid, and/or an HDAC inhibitor, such as a SCFA or SAHA, may, if desired, be presented in a bottle, jar, vial, ampoule, tube, or other container-closure system approved by the Food and Drug Administration (FDA) or other regulatory body, which may provide one or more dosages containing the active ingredient. The package or dispenser may also be accompanied by a notice associated with the container in a form prescribed by a governmental agency regulating the manufacture, use, or sale of pharmaceuticals, or the notice indicating approval by the agency. In certain aspects, the kit may include a formulation or composition as taught herein, a container closure system including the formulation or a dosage unit form including the formulation, and a notice or instructions describing a method of use as taught herein.

In some embodiments, the kit includes a container which is compartmentalized for holding the various elements of a formulation (e.g., the dry ingredients and the liquid ingredients) or composition, instructions for making the formulation or composition, and instructions for preventing, treating, or controlling cancer (e.g., liver cancer, colon cancer (e.g., colon cancer in a subject who has one or more colon polyps)) or a metabolic disease (e.g., diabetes, obesity, NASH, NAFLD). In some instances, kits of the present invention are used to treat colon cancer in a subject who has one or more colon polyps. In certain embodiments, the kit may include the pharmaceutical preparation in dehydrated or dry form, with instructions for its rehydration (or reconstitution) and administration.

Kits with unit doses of the active composition, e.g. in oral, rectal, transdermal, or injectable doses (e.g., for intramuscular, intravenous, or subcutaneous injection), are provided. In such kits, in addition to the containers containing the unit doses will be an informational package insert describing the use and attendant benefits of the composition in preventing, treating, or controlling cancer (e.g., liver cancer, colon cancer) or a metabolic disease. Suitable active compositions and unit doses are those described herein.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

### VI. Examples

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1. miR-22-Mediated Silencing of Histone Deacetylases and Triggering of Colon Cancer Cell Apoptosis by Retinoic Acid and HDAC Inhibitors

This example shows that all-trans retinoic acid (RA; also known as tretinoin) and HDAC inhibitors such as suberanilohydroxamic acid (SAHA) and the short-chain fatty acid butyrate induced colon cancer cell apoptosis through microRNA-22 (miR-22)-mediated silencing of histone deacetylases.

### Introduction

More than 95% of colorectal cancers develop from adenomas. Adenomatous polyps are lesions that can develop into colon cancer. It has been found that two microRNAs (miR-22 and miR-34a) can be unregulated by bacterial-generated short-chain fatty acids, retinoic acid (an active vitamin A metabolite), and bile acid (an important chemical for nutrient metabolism and absorption). When animals have dysbiosis (a condition characterized by an inbalance of gut microbiota), a lack of nutrients, or dysregulated bile acid synthesis, the expression level of those two microRNAs is reduced. Moreover, the expression level of these two microRNAs has been shown to be substantially reduced in liver and colon cancers. Furthermore, the levels of these two microRNAs are reduced in patients who have polyps. In particular, the levels of these two microRNAs have been shown to be lower in polyps compared to normal colonic specimens within the same individuals. These findings indicate that the microenvironment within the colon can be altered due to dysbiosis or dysregulated bile acid synthesis resulting, for example, from an unhealthy diet. Detection of these two microRNAs in polyps or colon and liver cancer specimens provides a clear indication that the pathological condition can be caused by dysbiosis and/or dysregulated bile acid synthesis, which can result, for example, from not eating healthy foods such as fiber. Detecting the expression levels of microRNAs provides information to help inform patients of the cause of detected polyps, enabling them to make informed choices about altering their dietary habits.

The findings presented here demonstrate, among other things, the importance of nutritional supplements and dietary interventions to prevent and treat colon cancer. RA and histone deacetylase (HDAC) inhibitors such as short-chain fatty acids, including butyrate, act through miR-22 to inhibit HDACs and induce apoptosis in colon cancer cells. The epigenetic regulation of retinoic acid receptor beta (RARE) transcription as mediated by miR-22 is a novel mechanism for the anti-carcinogenic effect of RA and HDAC inhibitors such as butyrate.

### Methods and Results

Retinoic Acid and Butyrate Synergistically Promoted Apoptosis and Induced RARβ and miR-22 Expression

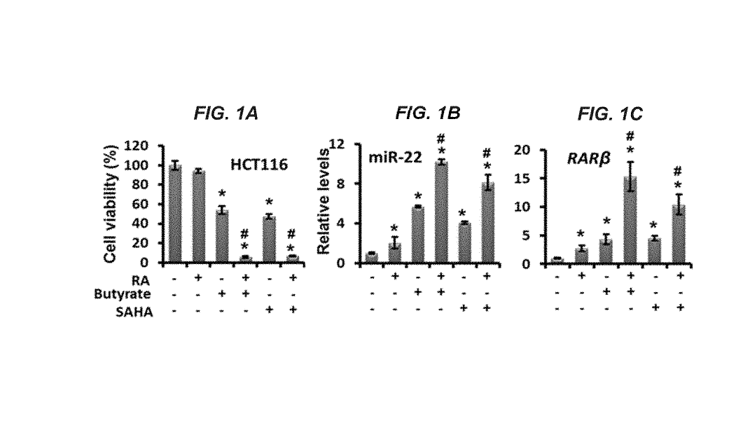

HCT116 cells were treated with retinoic acid (RA; 10 μM), butyrate (5 mM), SAHA (5 μM), or a combination of RA and butyrate or SAHA for 24 hours. SAHA (vorinostat) is an FDA-approved anti-tumor drug and was included as a positive control. Cell viability (shown in FIG. 1A) was measured using an MTT assay. Treatment with butyrate or SAHA alone reduced cell viability. Furthermore, treatment with butyrate and RA or SAHA and RA produced a synergistic effect. FIGS. 1B and 1C show the expression levels of miR-22 and retinoic acid receptor beta (RARβ), respectively, in HCT116 cells treated as described above. Expression was measured using qRT-PCR. Expression of miR-22 and RARB was upregulated by treatment with RA, butyrate, or SAHA alone. Furthermore, this upregulation was synergistically increased when RA was combined with butyrate or SAHA.

RA and Butyrate Regulate miR-22 Expression Through RARβ

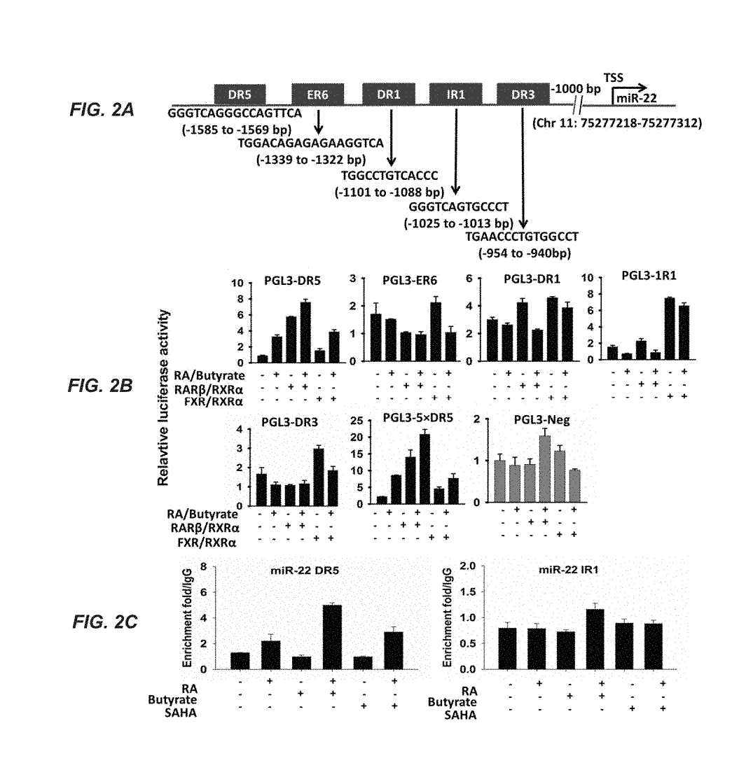

FIG. 2A shows a map of miR-22 promoters illustrating the location of each binding motif. A putative DR5, ER6, DR1, IR1, or DR3 motif was cloned into a PGL3 vector and HCT116 cells were subsequently co-transfected with a PGL3 vector construct and either RARB and retinoid X receptor alpha (RXRα) or farnesoid X receptor (FXR) and RXRα. Six hours post-transfection, cells were treated with DMSO or a combination of RA and butyrate for 24 hours. The results of luciferase activity assays following treatment are shown in FIG. 2B. PGL3-Neg and PGL3-5×DR5 were used as a negative control and positive control, respectively. FIG. 2C depicts fold enrichment of DR5 and IR1 binding using an anti-RARβ antibody in HCT116 cells as measured by ChIP-qPCR.

miR-22 Target HDAC4, SIRT1 and Cyclin A2

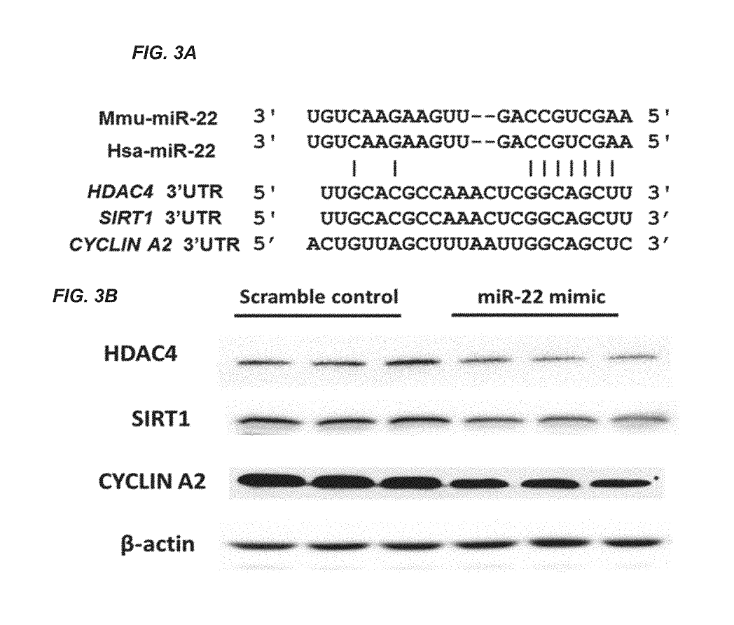

The sequence alignments depicted in FIG. 3A show that human and mouse variants of miR-22 can partially pair with the 3′UTRs of the genes encoding histone deacetylase 4 (HDAC4), sirtuin 1 (SIRT1) and cyclin A2 (HDAC4, SIRT1, and CCNA2, respectively). HCT116 cells were transfected with either an miR-22 mimic or a scramble control, and 48 hours later the protein levels of HDAC4, SIRT1, and cyclin A2 were measured (FIG. 3B). The expression of all three proteins was reduced by treatment with the miR-22 mimic.

Reversal of RA- and Butyrate-Induced HDAC and Cyclin A2 Inhibition by miR-22 Inhibitor

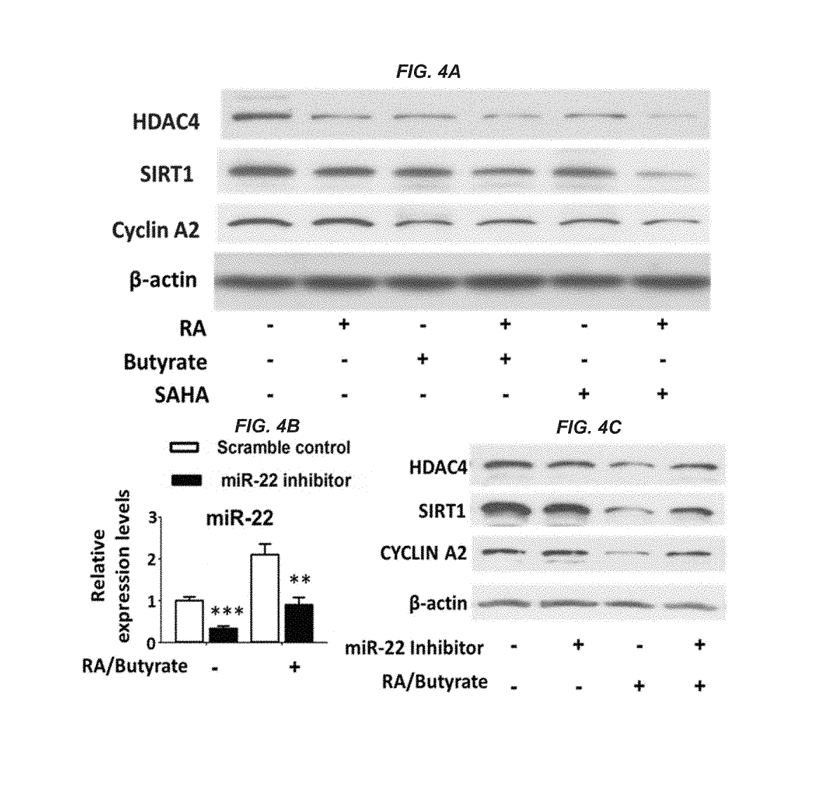

HCT116 cells were treated with RA (10 μM), butyrate (5 mM), SAHA (5 μM), or a combination of RA and butyrate or RA and SAHA, and the protein expression of cyclin A2 and the HDACs HDAC4 and SIRT1 was measured (FIG. 4A). The most striking reductions in protein expression were observed when a combination of RA and butyrate or SAHA were used for treatment. In separate experiments, cells were transfected with either an miR-22 inhibitor or a scramble control and treated with a combination of RA and butyrate or DMSO only. As shown in FIG. 4B, treating the cells with RA and butyrate increased the expression of miR-22, but this increased expression was blunted by incubation with the miR-22 inhibitor. As shown in FIG. 4C, protein expression of HDAC4, SIRT1, and cyclin A2 was inhibited by treatment with RA and butyrate, but this inhibition was reversed by co-treatment with the miR-22 inhibitor.

### RA and Butyrate Increased RARβ Transcription Through Histone Modification

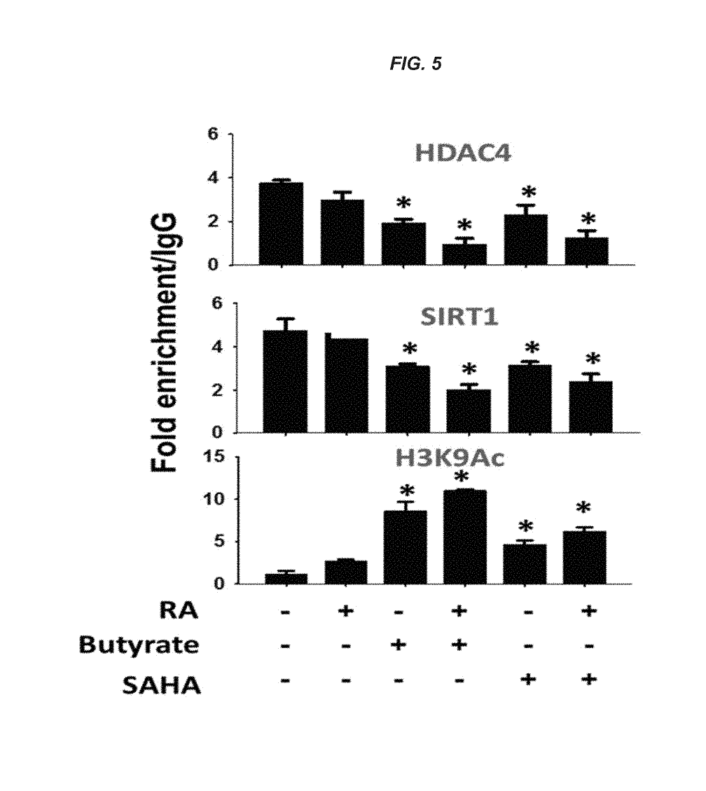

The results of ChIP-qPCR experiments are shown in FIG. 5. Treatment with butyrate (5 mM), SAHA (5 μM) or a combination of RA (10 μM) and butyrate or SAHA reduced the recruitment of HDAC4 and SIRT1. Furthermore, treatment with butyrate or SAHA alone, or a combination of RA and butyrate or SAHA, increased histone acetylation in the gene encoding RARβ (RABβ). The most pronounced effects were observed when a combination of RA and butyrate were used for treatment.

### Discussion

In these experiments, it was shown that all-trans retinoic acid (RA) and butyrate, which are normally present in the digestive tract, induce miR-22. RA is a biologically active metabolite of vitamin A and a natural agonist for the tumor suppressor retinoic acid receptor β (RARβ). Butyrate is a short-chain fatty acid and histone deacetylase inhibitor, produced by fiber-fed Gram-positive bacteria. Both agents can induce apoptosis in cancer cells. This work tested the hypothesis that RA and butyrate act through miR-22 to inhibit HDACs and induce apoptosis in colon cancer cells. These results showed that RA/butyrate combination treatment synergistically promoted apoptosis and potently induced RARβ expression compared to single compound treatment in colon HCT116 cancer cells. The induction of miR-22 by RA and butyrate was transcriptionally regulated by RARβ/RXRα direct binding to a DR-5 motif located at −1569 to −1585 bp upstream of miR-22, as demonstrated by transient transfection assay. Furthermore, miR-22 mimics also reduced the protein levels of HDAC4 and SIRT1, which were previously identified as miR-22 targets in rat cardiomyocytes. Consistently, RA and butyrate reduced HDAC4 and SIRT1 protein levels in HCT116 cells. In addition, ChIP-qPCR revealed that butyrate, but not RA, decreased the recruitment of HDAC4 and SIRT1 to the transcriptional regulatory region of the RARB gene (−53 to −36 from the transcription start site) in HCT116 cells. When RA was added in combination with butyrate, the recruitment of each protein deacetylase was further reduced. Moreover, butyrate, SAHA, and their combination with RA increased histone acetylation as revealed by ChIP using an anti-H3K9Ac antibody. These data showed that butyrate and RA increased RARB transcription through histone modification. Moreover, miR-22 inhibitors abolished the reduction of protein deacetylase levels induced by RA and butyrate.

In conclusion, a combination of retinoids (such as RA) and HDAC inhibitors (including SCFAs such as butyrate) induced miR-22 to promote apoptosis of colon cancer cells. miR-22-mediated inhibition of HDAC4 and SIRT1 is a novel pathway to explain the anti-carcinogenic effect of RARβ.

### Example 2. Inhibition of Colon Cancer Cells with Retinoic Acid and HDAC Inhibitors

This example shows that the viability of two different colon cancer cell lines was inhibited by treatment with retinoic acid (RA) and/or a histone deacetylase (HDAC) inhibitor. Furthermore, this example shows that a combination of RA and an HDAC inhibitor was more effective at inhibiting the viability of both cancer cell lines than treatment with the compounds individually.

### Methods

HCT116 and DLD-1 colon cancer cells were treated with dimethyl sulfoxide (DMSO) only (as a negative control), RA (10 μM), Fenretinide (10 μM), or an HDAC inhibitor. The HDAC inhibitors were selected from SAHA (5 μM) and a short-chain fatty acid (SCFA). SCFAs were selected from butyrate (5 mM), propionate (10 mM), and valerate (10 mM). Some HCT116 and DLD-1 cells were treated separately with a combination of RA and one of the SCFAs. Cells were treated for 48 hours before viability was assessed.

### Results

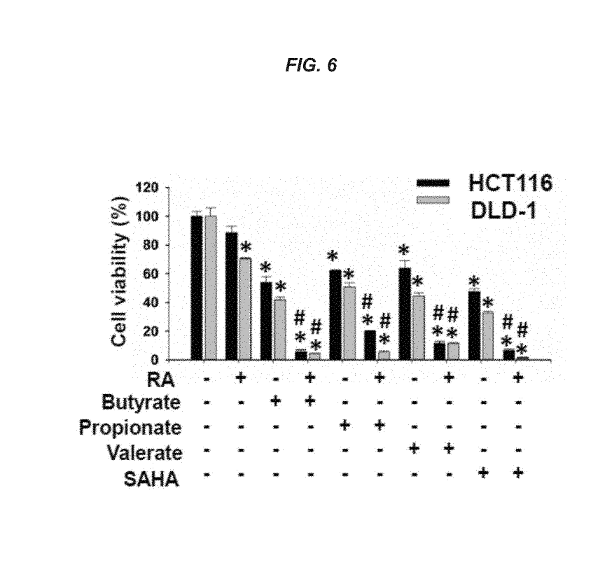

As shown in FIG. 6, treatment of DLD-1 cells with RA alone or any of the HDAC inhibitors alone reduced cell viability compared to DMSO control (p<0.05). A similar result was observed with the HCT116 cells. Furthermore, treatment with SAHA alone or any of the SCFAs alone significantly reduced cell viability (p<0.05).

FIG. 6 also shows that treating cells with a combination of RA and an HDAC inhibitor produced a synergistic effect. Treating either cell line with RA and an HDAC inhibitor produced a significant reduction in cell viability compared to DMSO control (p<0.05). Furthermore, combination treatments produced a striking reduction in cell viability compared to individual treatments alone (p<0.05).

In summary, these experiments showed that not only were HDAC inhibitors, including various SCFAs, effective at inhibiting colon cancer cell viability, but a remarkable synergistic effect was also observed when HDAC inhibitors were combined with a retinoid.

### Example 3. The Combination Effect of Short-Chain Fatty Acids and Retinoic Acid in Inducing miR-22 to Reduce Protein Deacetylation and Induce NUR77-Mediated Apoptosis of Colon Cancer Cells

This example shows that a combination of a retinoid and a short-chain fatty acid can induced apoptosis in colon cancer cells.

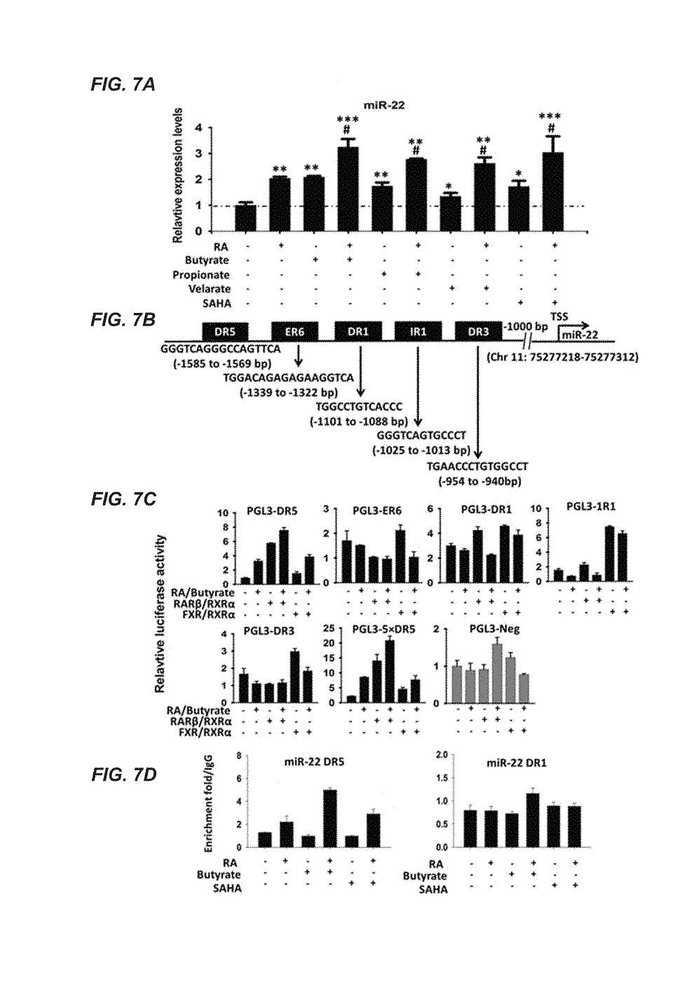

As shown in FIG. 7, retinoic acid (RA) and short-chain fatty acids (SCFAs) that have HDAC inhibitory effect induced miR-22 expression through the binding of RARβ to a DR5 motif. The expression level of miR-22 in HCT116 cells 24 hours after treatment with butyrate (5 mM), propionate (10 mM), valerate (10 mM), SAHA (5 μM), RA (10 μM), or a combination of RA and one of the SCFAs or SAHA is shown in FIG. 7A.

FIG. 7B shows a map of miR-22 promoters illustrating the location of each binding motif. A putative DR5, ER6, DR1, IR1, or DR3 motif was cloned into a PGL3 vector and HCT116 cells were subsequently co-transfected with a PGL3 vector construct and either a combination of RARB and retinoid X receptor alpha (RXRα) or a combination of farnesoid X receptor (FXR) and RXRα. Six hours post-transfection, cells were treated with DMSO or a combination of RA and butyrate for 24 hours. The results of luciferase activity assays following treatment are shown in FIG. 7C. PGL3-Neg and PGL3-5×DR5 were used as a negative control and positive control, respectively.

As shown in FIG. 7D, chromatin immunoprecipitation followed by qPCR (ChIP-qPCR) data showed that the combination effect of butyrate (5 mM) or SAHA (5 uM) plus RA (10 μM) was more effective than the single treatments for enhancing the binding of RARβ to a DR5 motif, but not the IR1 motif.

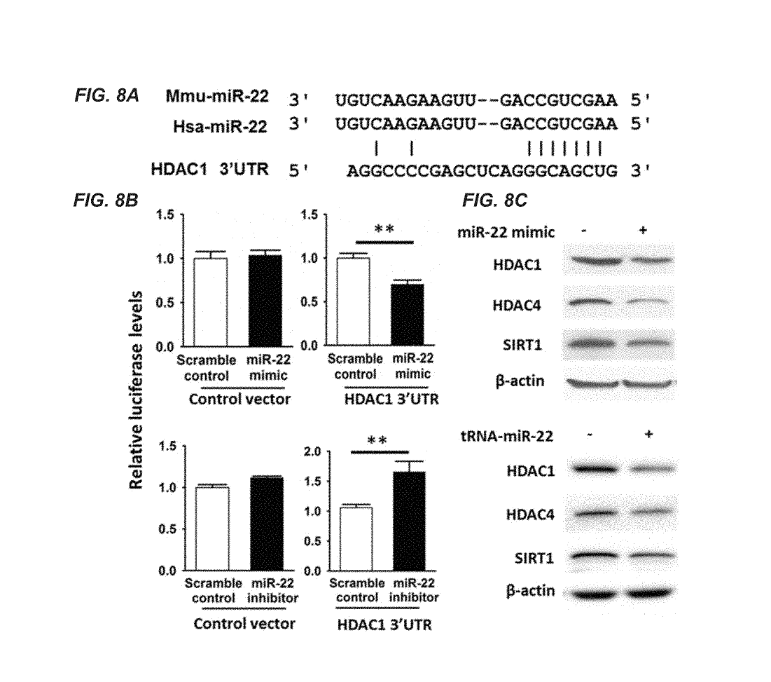

The sequence alignments depicted in FIG. 8A show that human and mouse variants of miR-22, which are conserved, can partially pair with the 3′UTR of the gene encoding histone deacetylase 1 (HDAC1). FIG. 8B shows the relative luciferase activity levels of HCT116 cells that were transfected with psiCHECK2-HDAC1, which contained the 3′UTR of HDAC1. Cells were also transfected with either miR-22 mimics or miR-22 inhibitors. Scramble controls were used as negative controls. FIG. 8C shows the protein levels of HDAC1, histone deacetylase-4 (HDAC4), and sirtuin 1 (SIRT1) as determined by Western blot in HCT116 cells that were transfected with either miR-22 mimics or scramble controls.

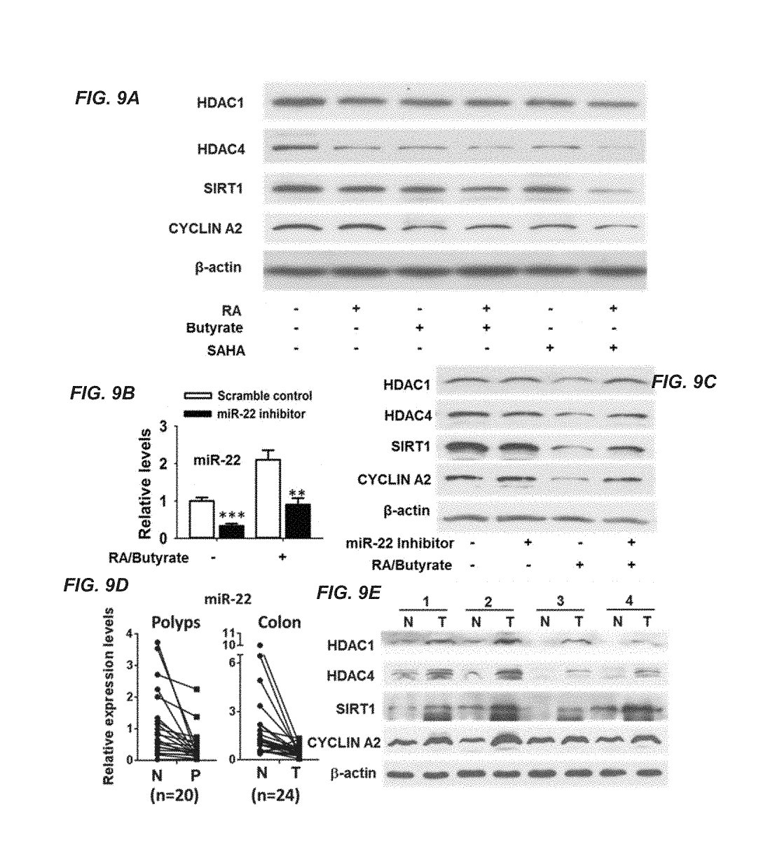

The reduced expression of HDAC1, HDAC4, SIRT1, and cyclin A2 by a combination of RA and butyrate was reversed by miR-22 inhibitors. FIG. 9A shows the expression levels of HDAC1, HDAC4, SIRT1, and cyclin A2 in HCT116 cells that were treated with RA (10 μM), butyrate (5 mM), SAHA (5 μM), or a combination of RA and butyrate or SAHA. As shown in FIG. 9B, the levels of miR-22 were reduced by treating HCT116 cells with the mir-22 inhibitor, compared to the scramble control. FIG. 9C depicts the protein expression levels of HDAC1, HDAC4, SIRT1, and cyclin A2 (with (3-actin as a control) in HCT116 cells 48 hours after transfection of miR-22 inhibitors or scramble controls. Cells were also treated with a combination of RA and butyrate, either alone or in combination with the mir-22 inhibitor or scramble control.

FIG. 9D shows the levels of miR-22 in polyps (P) and colon cancers (T) and their paired adjacent normal (N) tissues from 20 patients who had polyps, and another 24 patients who had colon cancer. The levels of HDAC1, HDAC4, SIRT1, and cyclin A2 (with (3-actin as a control) in colon cancers (T) and their adjacent benign (N) tissues from four patients who had colon cancers are shown in FIG. 9E.

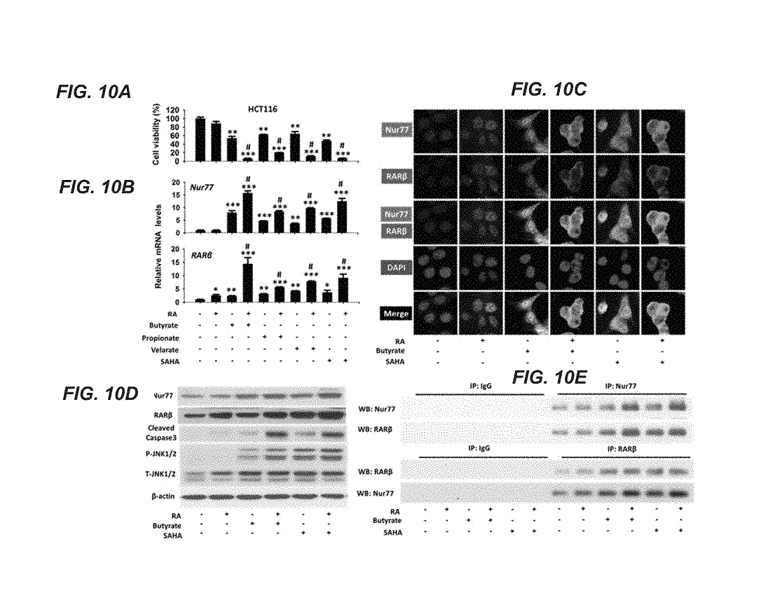

The combination of RA and HDAC inhibitors synergistically promoted apoptosis and the induction and interaction of NUR77 and RARB in HCT116 cells. FIG. 10A shows the viability of HCT116 cells that were treated with RA and/or butyrate, propionate, valerate, or SAHA for 48 hours. Cell viability was monitored by MTT assay. The relative mRNA expression of Nur77 and RARB for cells that were treated as above is shown in FIG. 10B. The levels of cell death were inversely correlated with the induction of NUR77 and RARB mRNA, which were quantified by qRT-PCR.

FIG. 10C shows the induction and co-localization of NUR77 and RARβ in HCT116 cells that were treated by RA and/or butyrate or SAHA. Cells were immunostained with antibodies specific to NUR77 or RARβ, followed by species appropriate Alexa Fluor secondary antibodies. The protein levels of NUR77, RARβ, cleaved Capspase 3, phospho (P)-JNK1/2, and total (T)-JNK1/2 were determined in HCT116 cells that were treated with RA and/or butyrate or SAHA (FIG. 10D). A combination of RA and butyrate or SAHA induced cleaved caspase 3 that was associated with JNK1/2 activation. FIG. 10E illustrates the expression levels of proteins extracted from HCT116 cells that were treated as indicated above. Proteins were immunoprecipitated by anti-NUR77 or anti-RARβ antibodies, or IgG, followed by Western blot using anti-RARβ or anti-NUR77 antibodies.

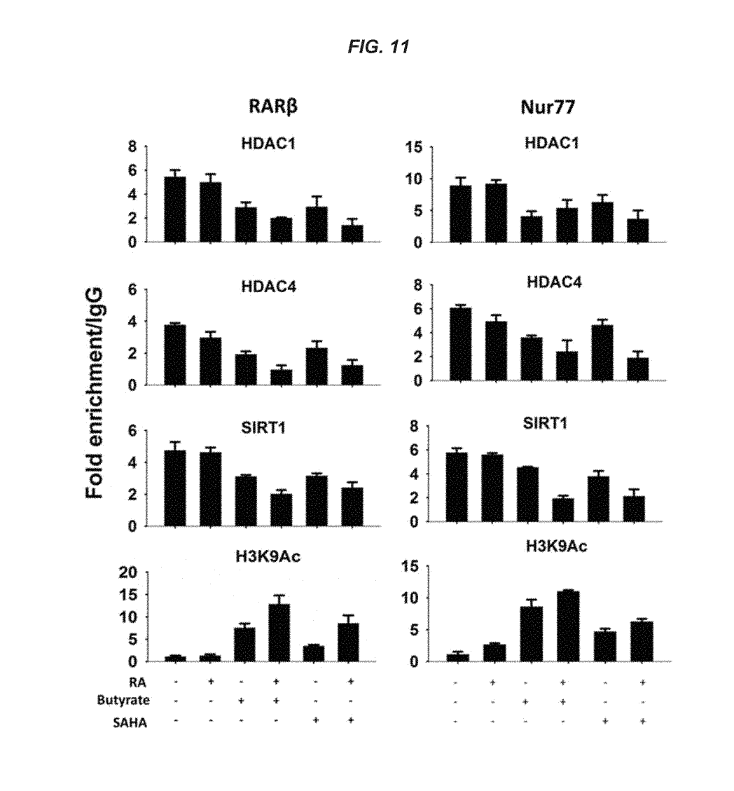

FIG. 11 shows the combination effects of RA plus the HDAC inhibitors butyrate and SAHA in reducing the recruitment of HDAC1, HDAC4, and SIRT1, as well as in increasing histone acetylation in the RARB and NUR77 genes. HCT116 cells were treated with RA (10 μM), butyrate (5 mM), SAHA (5 μM), or a combination of RA and butyrate or SAHA for 24 hours. Cell lysates were used for ChIP with antibodies specific for HDAC1, HDAC4, SIRT1, or acetylated H3K9, followed by qPCR using primers specific for the RARB and NUR77 genes. DMSO treated cells and IP using IgG were included as negative controls. □inding was expressed relative to the IgG negative controls.

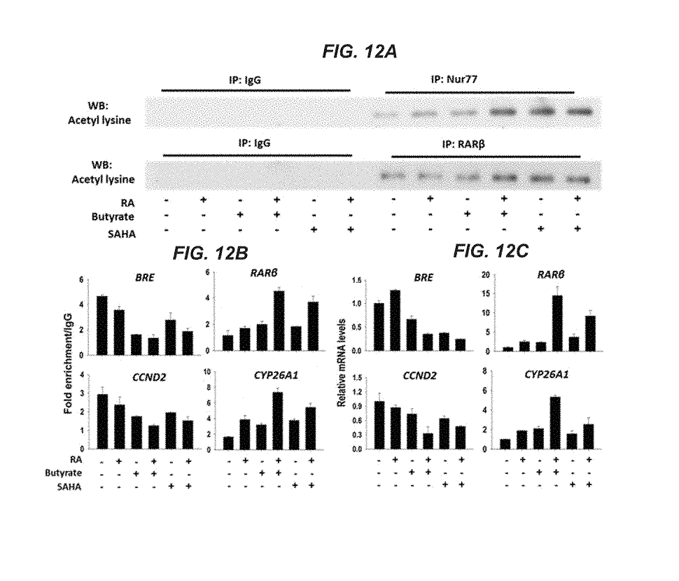

RA and butyrate-induced NUR77 acetylation was accompanied by reduced NUR77 transcriptional regulatory activity. FIG. 12A depicts the expression levels of proteins that were extracted from HCT116 cells and immunoprecipitated by anti-NUR77 and RARβ antibodies or IgG, followed by Western blot using an anti-acetyl lysine antibody. FIG. 12B illustrates ChIP-qPCR data that showed the binding of NUR77 and RARβ to their target genes. The mRNA levels of NUR77 target genes BRE as well as CCND2 and RARβ target genes RARβ and CYP26A1 in HCT116 cells are shown in FIG. 12C.

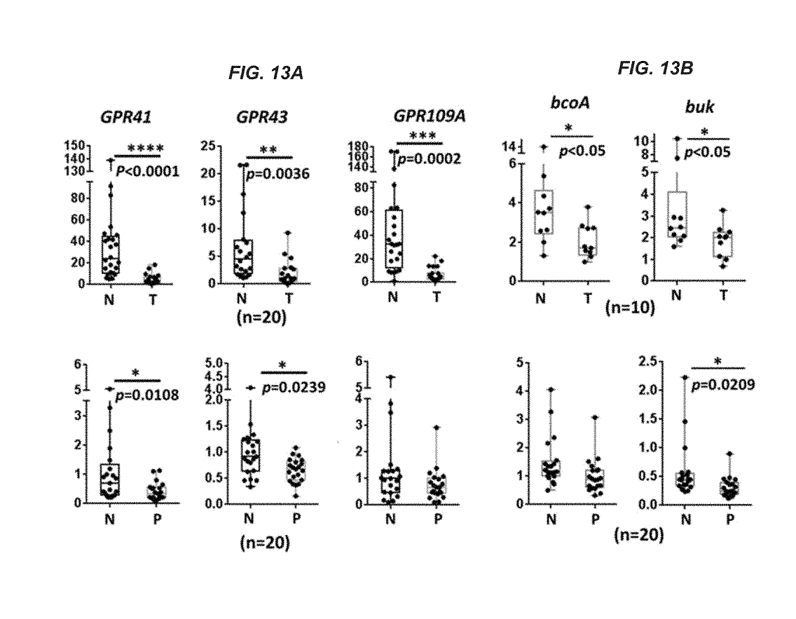

FIG. 13 illustrates that butyrate signaling was reduced in colon polyps and colon cancers. FIG. 13A depicts the mRNA levels of the short-chain fatty acid receptors GPR41, GPR43, and GPR109A in colon adenocarcinomas (T, N=20) and colon polyps (P, N=20) as well as their paired adjacent normal (N) specimens. FIG. 13B depicts the abundance of the butyrate-generating bacteria genes bcoA and buk in colon adenocarcinomas (T, N=10) and colon polyps (P, N=20) as well as their paired adjacent normal (N) specimens.

### Example 4. Retinoids and HDAC Inhibitors for the Treatment of Colon and Liver Cancers

### Identification and Significance of the Problem

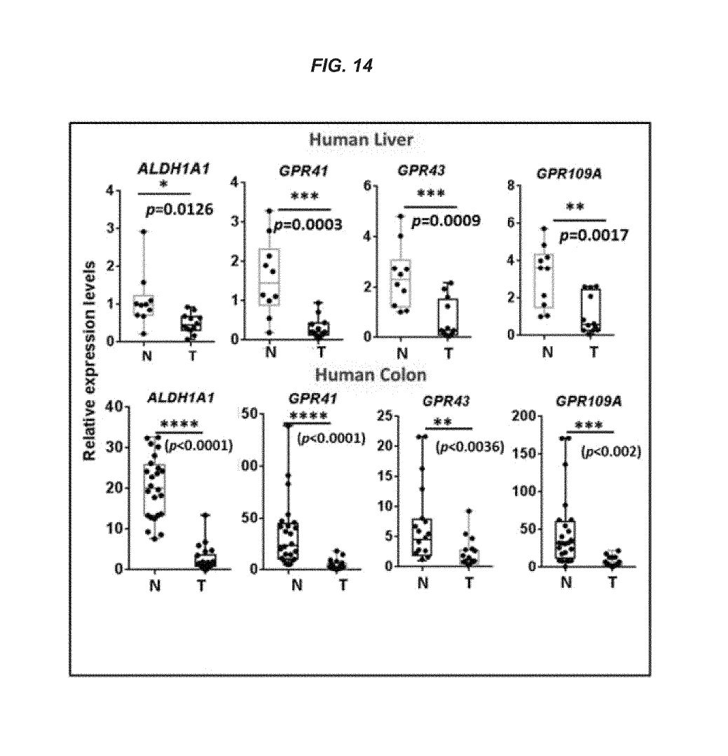

The incidence of liver cancer is rising because of obesity and metabolic syndrome. The only FDA-approved drug for liver cancer is sorafenib, which increases the survival in primary liver cancer by only 6 months with many side effects. There is an urgent need to develop alternative treatments for liver cancer. Our data revealed the combination effect of retinoids and histone deacetylase (HDAC) inhibitory property in inducing the apoptosis of liver cancer cells. Jointly those two types of chemicals also reduce inflammation and improve metabolism as well as insulin sensitivity, which are huge risks for liver carcinogenesis. The interactive health benefits of retinoids and HDAC inhibitors can be a natural way for the human body to fight liver cancer because some of those chemicals we studied, such as all-trans retinoic acid (RA) and short-chain fatty acids (SCFAs), are naturally present in the digestive tract. This application studies the combination of beneficial effects of retinoids and SCFAs. The SCFAs we refer to in this application are those that have HDAC inhibitors. We focus on butyrate and propionate because of their high concentrations found in the human body. Excitingly, our novel findings revealed that the mRNA levels of aldehyde dehydrogenase (ALDH1A1), encoding an enzyme generates RA, as well as the receptors for SCFAs (GPR41, GPR43, and GPR109A), were reduced in hepatocellular carcinoma (HCC) and colon cancer specimens suggesting the significance of those signaling pathways in the liver and colon (FIG. 14).

### Retinoids and Cancer

The effect of retinoids on cancer prevention and treatment has received a lot of attention. Altered expression of RA receptors is associated with malignant transformation. In addition, retinoids suppress carcinogenesis in tumorigenic animal models for skin, oral, lung, breast, bladder, ovarian, and prostate [1-7]. In humans, retinoids reverse premalignant skin lesions, effectively induce the differentiation of myeloid cells, and prevent lung, liver, and breast cancer [8-12]. However, retinoids have side effects and RA syndrome is a life-threatening complication in patients treated with RA. This syndrome is characterized by dyspnea, fever, weight gain, hypotension, and pulmonary infiltrates. Thus, there is a need to develop alternative treatments in order to reduce toxicity, which is one of the goals of this project.

### A Unique Quality of a Synthetic Retinoid

N-(4-hydroxyphenyl) retinamide (fenretinide) is a synthetic retinoid and one of the most promising clinically tested retinoids. The modification of the carboxyl end of all-trans RA with an N-4-hydroxyphenyl group increases apoptotic efficacy and reduces side effects in comparison with other retinoids [13]. In addition, fenretinide does not induce point mutations or chromosomal aberrations and is not genotoxic [14]. The safe quality of fenretinide has been extensively examined in humans. The key findings are summarized here: (1) Large breast cancer chemoprevention study (3000 patients, 5 year study) demonstrating fenretinide to be well tolerated in humans; (2) NCI case report confirming tumor response in an advanced cutaneous T-cell lymphoma patient; (3) The effect on cutaneous T-cell lymphoma and angioimmunoblastic T cell lymphoma were confirmed, and (4) Phase II clinical study showing fenretinide to be well tolerated in patients with small cell lung cancer as well as a stabilization of the disease in 24% of the patients [15]. These qualities suggest that fenretinide is suitable for long-term usage. SciTech Development, LLC has generated a formulation that has improved solubility (ST-001). Fenretinide is currently in the public domain and the NCI has specifically made the Good Manufacturing Practice Guide for Active Pharmaceutical Ingredients available to SciTech without charge for further study. One of the goals of this project is to improve the bioactivity of fenretinide using natural chemicals.

Retinoids and HDAC Inhibitors:

Retinoids exert their biological effects via transcriptional regulation controlled by retinoic acid receptors (RARs) and retinoid x receptors (RXRs) [16, 17]. HDAC inhibitors can epigenetically enhance the transcriptional activity of those nuclear receptors. HDAC inhibitors are used in clinics to combat cancer. Our data revealed that a combination of chemicals that have retinoid and HDAC inhibitory properties can effectively induce apoptosis of liver and colon cancer cells. In addition, they can jointly improve metabolism and insulin sensitivity to reduce cancer risks. Furthermore, they both have anti-inflammatory effects and can increase the expression of tumor suppressor miR-22, which inhibits multiple HDACs. Furthermore, our exciting data revealed that bacterial-generated SCFAs that have HDAC inhibitory property such as butyrate, propionate, and valerate have the same effect in inducing apoptosis and miR-22. The long-term goal of the current study is to use dietary modification to shift gut microbiota in order to produce specific SCFAs, thereby enhancing the effect of retinoids to combat cancer in the digestive tract.

Because the tested HDAC inhibitors (i.e. SCFAs) are naturally generated from gut bacteria fermentation, this combination therapy we propose can be a natural mechanism by which human body can combat metabolism-associated digestive cancer. The current study focuses on liver since there is no effective treatment available for liver cancer other than surgery. Since the chemicals we studied are present in the gut and liver, it is likely that the same principle can be applied to colon cancer.

### Technical Objectives

Sorafenib prolongs the survival in primary liver cancer by only 6 months with many side effects. This outcome does not justify its high price, at up to $5,000 per patient per month, and other countries have refused to give sorafenib authorization for liver cancer treatment. Using a drug like sorafenib, liver cancer will never be treated; it can only be controlled at most for a couple months. An effective cancer treatment strategy should target the pathways by which cancer arises in the first place. It is important to note that the liver is constantly exposed to all types of chemicals generated from the gut because more than 70% of hepatic blood comes from the gut via enterohepatic circulation [18]. Additionally, emerging evidence reveals that gut microbiota are not only implicated in colon cancer, but also affect hepatic inflammation and liver carcinogenesis. The proposed technical objective of this project is to examine the interactive effects of natural chemicals in the gut-liver axis for liver cancer treatment, which is logical and innovative.

There are only a few FDA-approved HDAC inhibitors. Vorinostat (SAHA) and Romidepsin are approved for cutaneous T-cell lymphoma. Panobinostat is approved for refractory multiple myeloma. All those chemicals have side effects. The human body via gut bacterial fermentation naturally generates SCFAs that have HDAC inhibitory property. The effect of HDAC inhibitors should be tested in the liver, as certain SCFAs such as propionate can be efficiently taken up by the liver. Our data already revealed the novel interactive relationship between retinoids and SCFAs. For example, butyrate stimulates RA production in the gut dendritic cells. Moreover, RA and butyrate enhance the expression of each other's receptors. We also uncovered the novel effects of retinoid and SCFAs in inducing tumor suppressor miR-22, which inhibits multiple HDACs; this can be the endpoint assay to monitor the efficacy of the combination treatment. Regarding the treatment strategy, we will use a combination of retinoids that include both natural chemical all-trans RA and a synthetic retinoid fenretinide, which has very low toxicity, plus diet that modulates gut microbiota to produce specific SCFAs. Such novel treatment strategy is guaranteed to have low toxicity. Moreover, understanding the cross talk between genetic and epigenetic transcriptional regulators helps us understand how the effect generated from microbiota and host is coordinated. Together, the objective is to use of combinational diets, probiotics, and prebiotics, decreasing the dosage of drugs and thus reducing unwanted side effects.

### Work Plan

### Aim 1: Study the Combination Effect of Retinoids and SCFAs in Liver Cancer Cells

### Related Research for Aim 1

Commensal microbiota through fermentation of dietary fiber generate propionate, butyrate, and valerate, which all exhibit a HDAC inhibitory and anti-inflammatory properties that can combat cancer [19, 20]. SCFAs are also present in dairy products such as milk and cheese. SCFA-generating microbiota play a crucial role in energy harvesting and metabolism for colonocytes renewal. SCFAs are associated with increased satiety and reduced food intake [21, 22]. Transplant of gut microbiota from lean donors to patients with metabolic syndrome increased the abundance of butyrate-generating bacteria and improved insulin sensitivity [23]. T2DM patients have a reduced proportion of butyrate-producing Clostridiales (Roseburia and Faecalibacterium prausnitzii) [24, 25]. In addition, vancomycin removal of Gram-positive bacteria reduced insulin sensitivity in obese people [26]. Furthermore, SCFAs are implicated in cancer. Intestinal butyrate and butyrate-generating bacteria are consistently reduced in colon cancer patients and patients who have liver cirrhosis [27-30]. Thus, SCFAs provide benefits ranging from metabolic facilitation to cancer prevention, which are against the negative outcomes of Western diet consumption.

### The Interactions Between RA and SCFAs

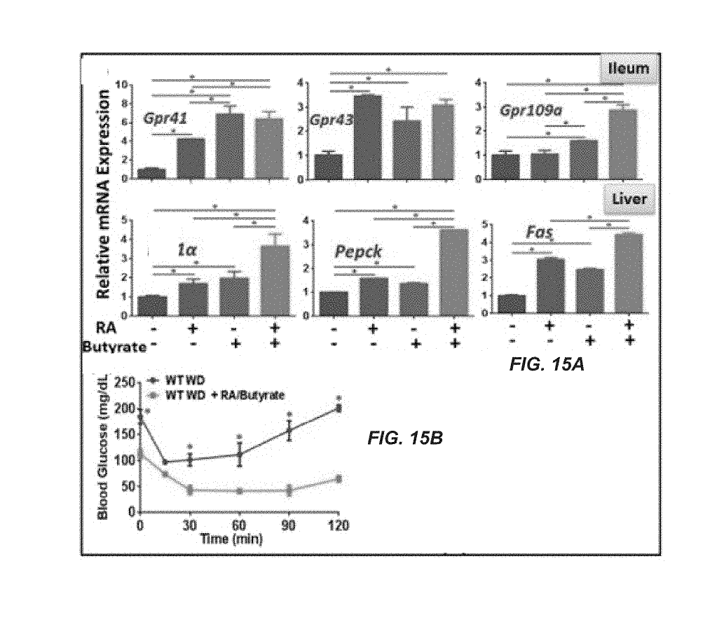

Our data showed the interacting effects of SCFAs and RA. Butyrate and RA alone could induce the expression of receptors for SCFAs Gpr41 and Gpr43, but a combination of both did not further increase their expression in mice (FIG. 15A). In addition, butyrate, but not RA, induced Gpr109a (the receptor for butyrate) and a combination of both was even more effective than butyrate alone (FIG. 15A). This might be the first evidence suggests that RA may be able to boost SCFA effects by increasing their receptors. Moreover, butyrate increases the expression of ALDH1A1 in CD103+ dendritic cells in the gut to promote RA production [31-33]. The interactive metabolic beneficial effects of RA and butyrate were revealed. Our data revealed that combined treatment was more effective than a single chemical in inducing hepatic Pgc1α, Pepck, and Fas in mice (FIG. 15A), which suggested increased metabolism, gluconeogenesis, and fatty acid uptake. Moreover, butyrate plus RA substantially improved insulin sensitivity, thereby reducing the risk for cancer (FIG. 15B). Moreover, butyrate has anti-inflammatory effects; butyrate induces IL-10 expression in dendritic cells in a GPR109a-dependent manner [31]. Further, GPR109a induces IL-18, which suppresses inflammation-associated cancers [31, 34].

RA and HDAC Inhibitors Induce miR-22 and Reduce Protein Deacetylases

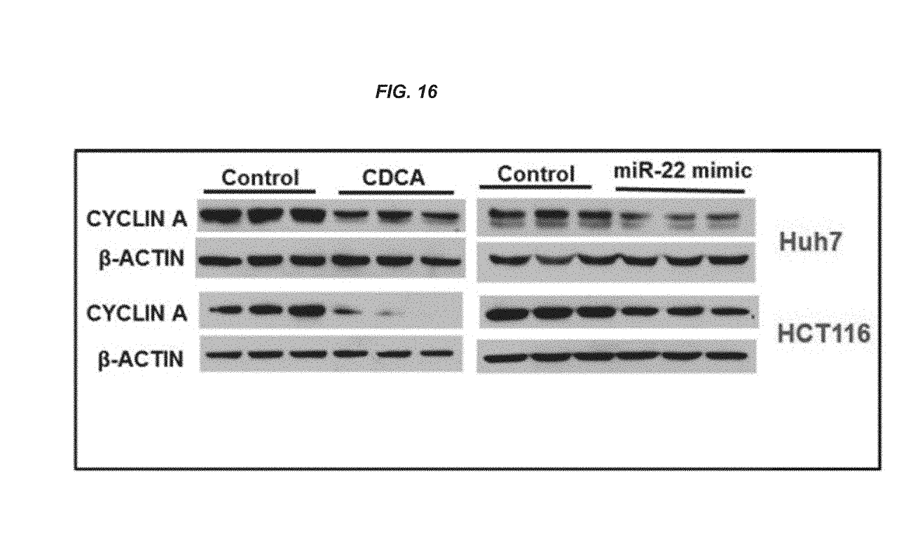

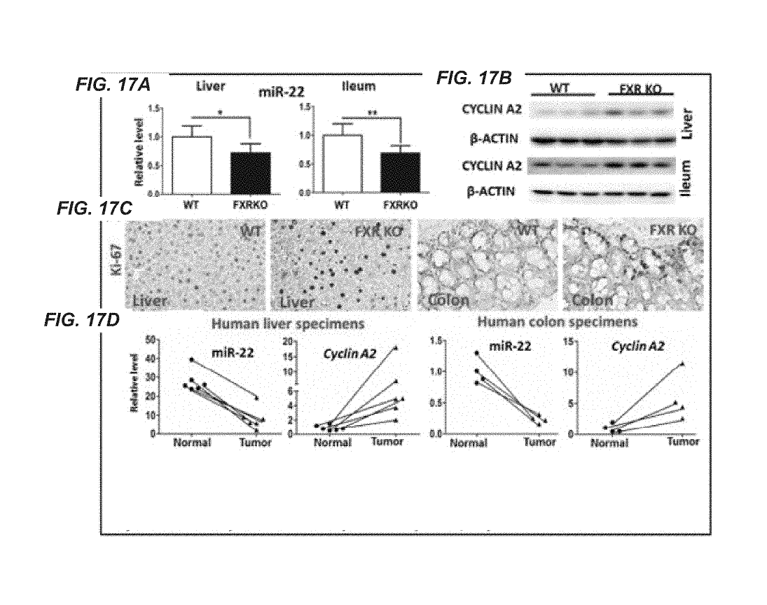

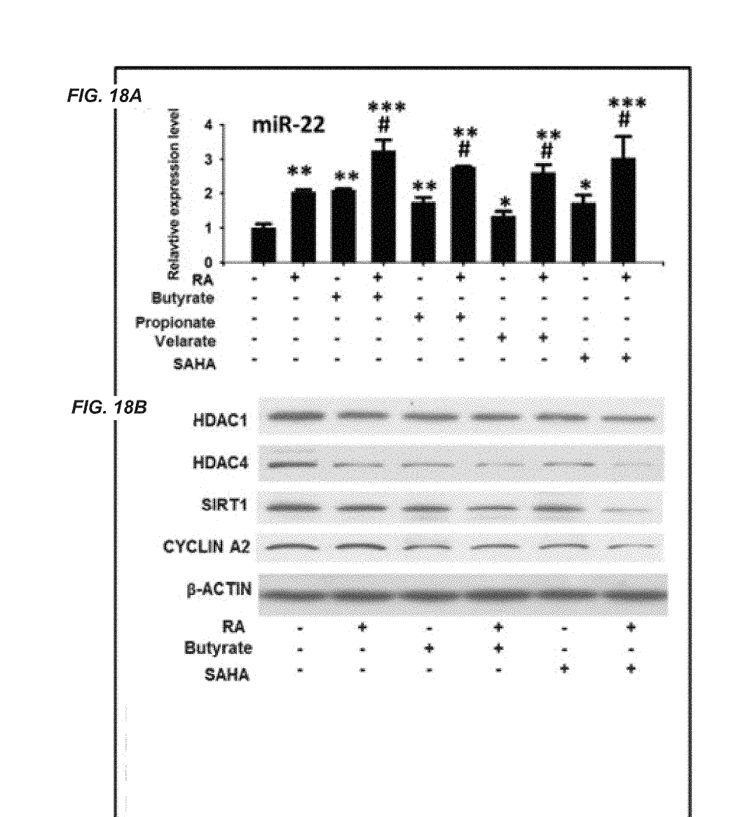

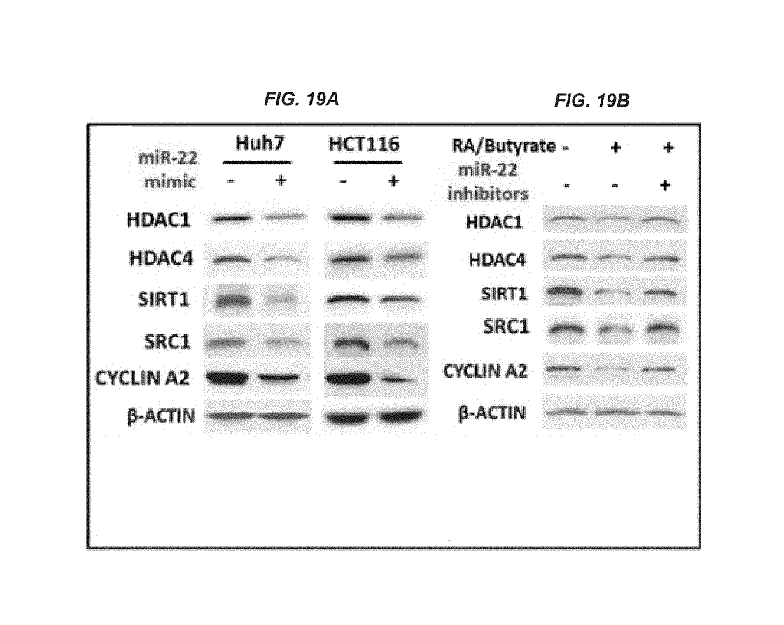

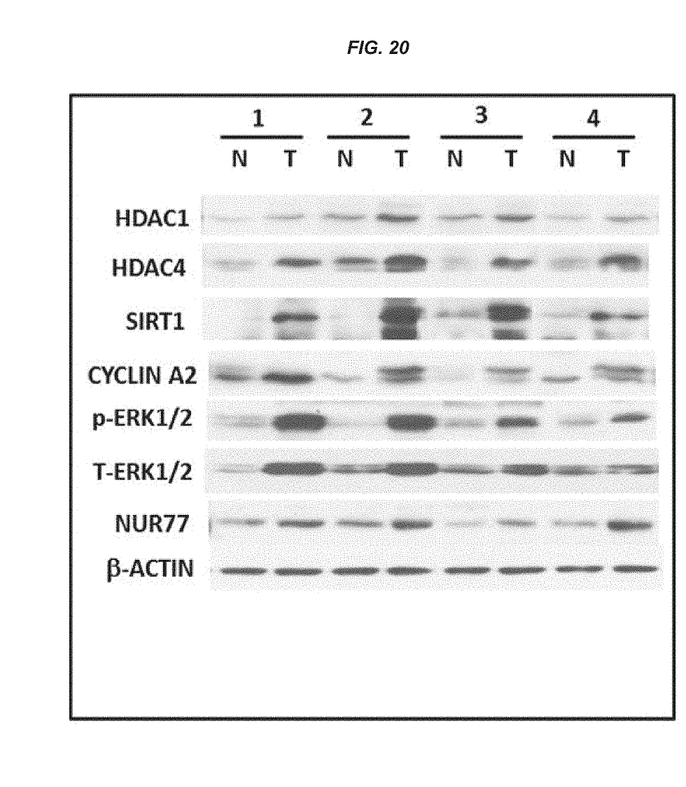

Due to the significant tumor suppressive role of miR-22, we have studied the regulation of miR-22 [35]. Our published data showed that chenodeoxycholic acid (CDCA), which has the highest binding affinity to FXR, the bile acid receptor, induces miR-22. In addition, our unpublished data showed that obetacholic acid is also effective in inducing miR-22 [35]. In a Phase II clinical trial, obetacholic acid improves insulin sensitivity and reduces liver inflammation as well as fibrosis in T2DM and NAFLD patients [36]. In dissecting the miR-22 downstream effect, we have discovered CYCLIN A2 as a brand new miR-22 target [35]. CDCA treatment and miR-22 mimics reduced CYCLIN A2 levels in liver Huh7 and colon HCT116 cancer cells (FIG. 16) [35]. miR-22 mimics also increased the number of G₀/G₁ Huh7 and HCT116 cells, supporting a role for miR-22 in cell cycle inhibition [35]. In contrast to the activation of FXR, FXR KO mice had reduced miR-22 and increased CYCLIN A2 as well as Ki-67 positive cells in liver and ileum, indicating the pathways we studied are commonly found in the digestive tract (FIGS. 17A and 17B). This can be one of the mechanisms by which FXR KO mice develop liver cancer spontaneously and have increased susceptibility to colon cancer [37-41]. In humans, the levels of miR-22 and Cyclin A2 were inversely correlated in liver and colon cancers (FIG. 17C). Taken together, this is the first study to show that miR-22, a tumor suppressor, is regulated by BA-activated FXR [35]. Moreover, our unpublished data showed that RA and three SCFAs, as well as SAHA, could induce miR-22 (FIG. 18A). Moreover, RA plus butyrate or SAHA inhibited the protein levels of HDACs, SIRT1, and CYCLIN A2 (FIG. 18B). The tumor suppressive effect of miR-22 has been demonstrated in different models [42-49]. Our novel data showed that miR-22 inhibited multiple HDACs such as HDAC1, HDAC4, and SIRT1 in liver Huh7 and colon HCT116 cancer cells (FIG. 19A). Moreover, miR-22 inhibitors prevented the reduction of HDAC1, HDAC4, SIRT1, and CYCLIN A2 caused by RA and butyrate treatment (FIG. 19B). Thus, it is likely that miR-22-inhibited HDAC is a mechanism by which RA and butyrate together exert their anti-cancer effects. Conversely, it is possible that dysbiosis-associated SCFA deficiency reduces miR-22, thereby increasing HDACs as a mechanism for carcinogenesis. This scenario is supported by our exciting novel data that reduced miR-22 in HCCs was accompanied by increased CYCLIN A2 and protein deacetylases HDAC1, HDAC4, and SIRT1, as well as activated p-ERK1/2 (FIGS. 17C and 20).

### Retinoids and HDAC Inhibitors Induce RARβ and NUR77-Dependent Cell Apoptosis

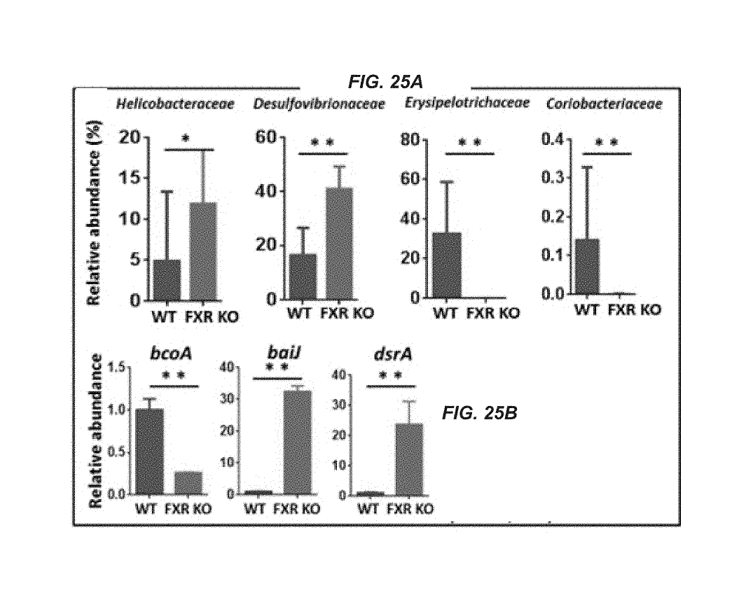

High fat diet increases secondary BAs deoxycholic acid (DCA) and lithocholic acid (LCA), which have known pro-inflammatory, DNA damaging, and tumor promoting effects as shown in many published papers including ours [35]. DCA and LCA are generated via 7-α dehydroxylation of primary BAs using bacteria such as Clostridium scindens-generated enzyme [50]. This finding clearly implicated the role of microbiota and secondary BAs in liver carcinogenesis [51]. Similarly, FXR KO mice, which have elevated BAs and develop spontaneous liver cancer, also have increased abundance of the BaiJ (BA inducible operon J) encoding enzyme that generates secondary BAs (FIG. 25B). Our published data showed that a long-term exposure to DCA and LCA increases the number of BA-resistant cells that had elevated nuclear NUR77, which is an oncogene overexpressed in liver and colon cancers, making NUR77 an attractive target for prevention and therapy [52, 53]. Our published data, as well as data presented in FIG. 20, showed that NUR77 is overexpressed in human HCCs [54]. Like RARβ, NUR77 is a nuclear receptor that controls transcription [54-56]. The transcription of the NUR77 can be activated by mitogens like epidermal growth factor (EGF) to induce cell proliferation [57]. However, NUR77 has dual roles in survival and death, and its intracellular location dictates these opposing effects [53]. Apoptosis inducers not only induce NUR77, but also nuclear export it, such that cytosolic NUR77 converts BCL-2 from an anti-apoptotic into a pro-apoptotic molecule [58].

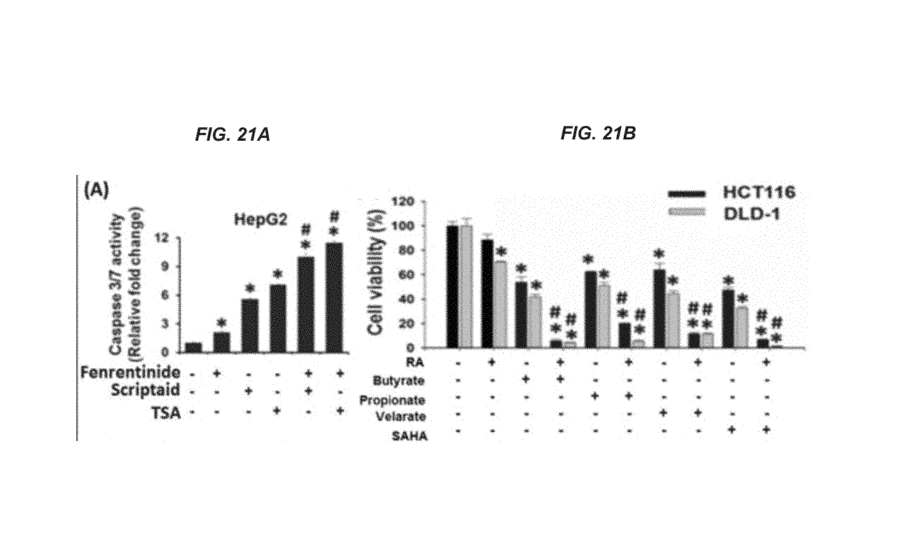

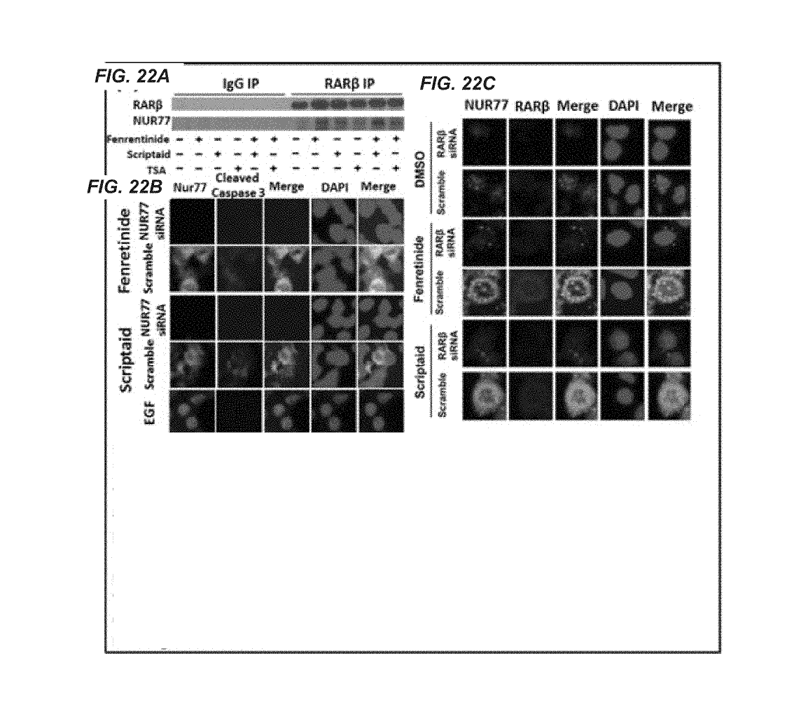

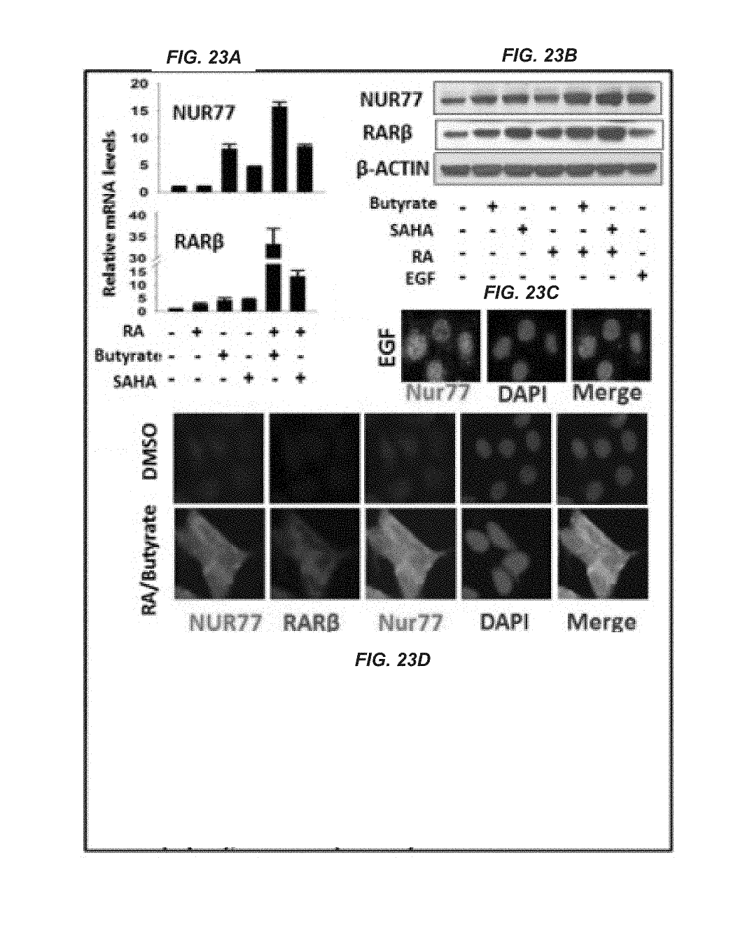

We have published a series of studies that showed fenretinide effectively induces apoptosis of liver cancer cells via a RARβ and NUR77-dependent mechanism [59]. In addition, the apoptotic effect is enhanced when a combination of retinoids plus HDAC inhibitors was used [59]. RA alone was not as effective as fenretinide in inducing apoptosis of liver cancer cells, but apoptosis occurs when HDAC inhibitors were combined with RA. Thus far, we have tested HDAC inhibitors trichostatin (TSA) [59], scriptaid [59], SAHA, and 3 SCFAs in inducing liver and colon cancer cell death (FIG. 21). Examples of those findings are shown in FIG. 21 [59]. Upon fenretinide and HDAC inhibitor treatment, RARβ and NUR77 were induced and could be co-precipitated (FIG. 22A). Moreover, fenretinide and scriptaid-induced apoptosis was NUR77-dependent, and cytosolic NUR77 became undetectable when RARβ was knocked down by siRNA (FIGS. 22B and 22C). We subsequently tested the effect of RA plus SCFAs to avoid using synthetic chemicals. Consistently, RA and butyrate or SAHA jointly induced NUR77 and RARβ mRNA and protein. They also exported those nuclear receptors to cytosol and induced apoptosis. In contrast, EGF induced nuclear NUR77, but not RARβ, to exert its proliferative survival effect (FIGS. 23A-23D). Based on these exciting findings, we propose to study the combination effects of retinoids and SCFAs in inducing of apoptosis of liver cancer cells.

### Methodology and Analyses for Aim 1

We have studied the effect of fenretinide in inducing apoptosis of liver cancer cells using various liver cancer lines, including Huh7, Hep3B, and HepG2. HepG2 was the only line resistant to the apoptotic effect of fenretinide. However, this resistance could be overcome by adding HDAC inhibitors or ERK1/2 inhibitor shown in our publications [59, 60]. To compare the efficacy of fenretinide plus butyrate and propionate, we propose to perform time course and dose response experiments in all 3 lines to determine the minimal dose and time required for fenretinide or RA alone or in combination with SCFAs to induce apoptosis. The treatment groups include 2 retinoids and 2 SCFAs individually or in combination plus vehicle control (DMSO). SAHA will be included as a positive control for comparison. The studied dose range will be 10 nM-10 μM for retinoids and 5-80 mM for butyrate and propionate. The biological dose of RA in human serum is 1-10 nM [61, 62] and butyrate and propionate is 20-40 mM in the gut [63, 64].

The following assays will be performed as the endpoints to evaluate the success of the treatment. TUNEL assay and Western blot for quantification of cleaved caspase 3 will be done to monitor the level of apoptosis. The expression of receptors for RA and SCFAs will be quantified by real-time PCR. The level of miR-22 induction will be monitored. Western blot will be done to quantify the level of HDAC1, HDAC4, and SIRT1, as well as CYCLIN A as the downstream of miR-22. HDAC Activity Assay Kit (Cayman Chemical) will be performed to monitor the overall HDAC activity. The protein level RARβ and NUR77 will be quantified by Western blot, and the intracellular location of those proteins will be studied by immunohistochemistry. Moreover, we will study the expression of genes that control hepatic metabolism. Examples include PEPCK, G6PASE, and FBP1, which are the rate limiting enzymes for gluconeogenesis. Fatty acid synthesis (SREBP1), translocation (CD36), and oxidation (CYP4A10) will be monitored. The expression of hepatic FGF21 and PGC1α, the key metabolism regulators, will be studied as well. We will also study the level of inflammatory genes such as IL-1β, IL-6, IL-10, IL-18, and TNFα in the liver, as they are implicated in digestive diseases [64].

Relationship with Future R&D for Aim 1

It is expected that RA may have a stronger effect than fenretinide in metabolism or anti-inflammation, but fenretinide definitely is a much better apoptosis inducer than RA [65]. We anticipate the best outcomes will be observed when combination treatments are used especially in miR-22 induction and HDAC inhibition. Three cell lines are included to ensure the observed effects are not cell line specific. Other liver cancer lines available from ATCC such as SNU398 and PLC/PRF/5 will be considered for additional data validation. It is also possible the propionate may have a better outcome than butyrate because the liver effectively uptakes propionate [66, 67]. In unlikely event that the apoptosis effect is not robust, the alternative approach is to include a low dose of SAHA and study the combined effect of retinoids, SCFAs, and low dose SAHA. The goal is to avoid using high doses of toxic chemicals such as synthetic HDAC inhibitors or RA. The best combination will be selected for the liver cancer treatment study proposed in Aim 2.

### Aim 2: Study the Combined Effect of Retinoids and Prebiotic or SCFA in Liver Cancer Treatment in Mice.

### Related Research for Aim 2

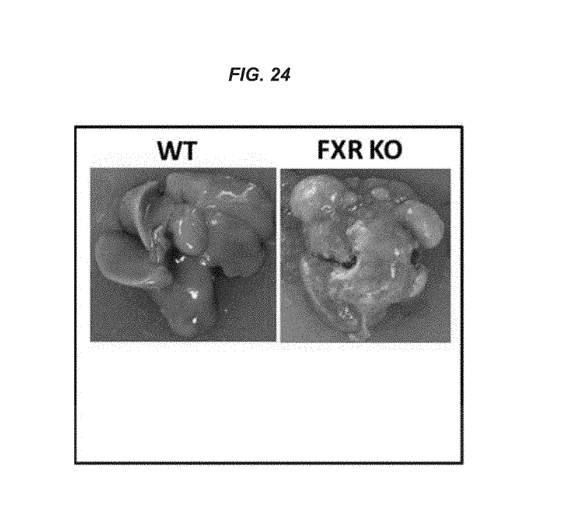

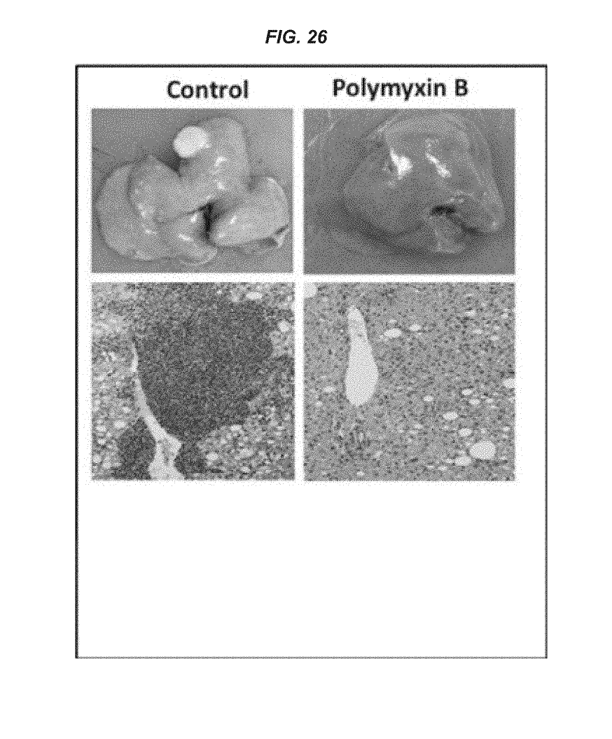

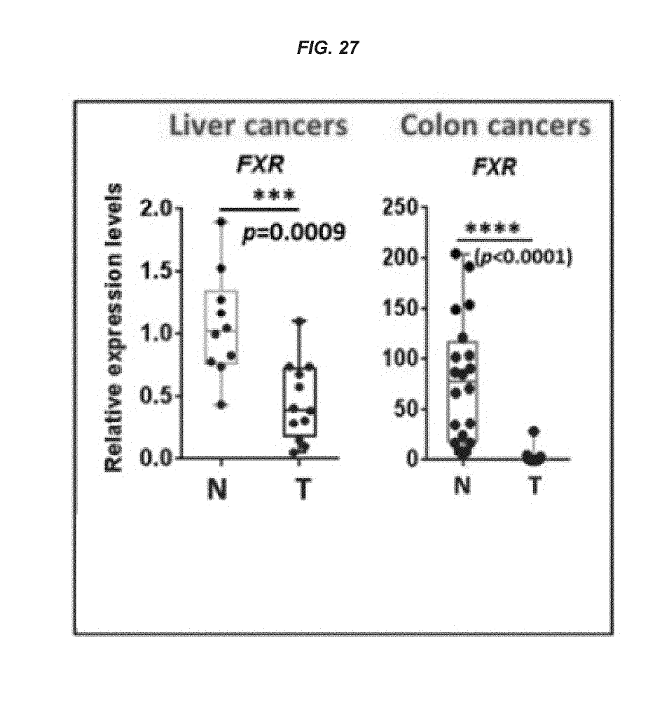

Emerging evidence reveals that dysbiosis associated dysregulated BA synthesis caused by Western diet consumption contributes to liver cancer [68]. FXR KO mice possess those features and are ideal models for liver cancer treatment. FXR KO mice have increased insulin resistance accompanied by elevated BAs with increased DCA and develop liver cancer when they are about 15 months old (FIG. 24) [69]. Treating FXR KO mice with cholestyramine to deplete BAs prevented liver cancer formation [38]. Moreover, data generated from pyrosequencing of tagged 16S rRNA gene amplicons revealed dysregulated BA synthesis is accompanied by dysbiosis. Specifically, our data showed FXR KO mice had increased abundance of Helicobacteraceae that can induce gastric cancer and Desulfovibrionaceae, which produce hydrogen sulfide, a gut barrier breaker [70, 71]. In addition, FXR KO mice also had reduced Erysipelotrichaceae and Coriobacteriaceae, which have many beneficial health effects [72, 73] (FIG. 25A). Consistently, FXR KO mice had reduced bcoA, which generates butyrate, and increased baiJ and dsrA that generate DCA and hydrogen sulfide, respectively (FIG. 25B). Moreover, 10 months old Western diet-fed FXR KO mice developed severe steatohepatitis with massive hepatic lymphocyte infiltration, which could be eliminated by polymyxin B. This exciting finding clearly indicated the role of Gram-negative bacteria in contributing to hepatic inflammation (FIG. 26). Furthermore, FXR is markedly reduced in patients with liver cirrhosis as well as liver and colon cancer, making FXR KO mice clinically relevant models [39, 69, 74]. Consistently, our data showed FXR mRNA levels were reduced in human liver and colon cancers (FIG. 27). Because it takes about 10-15 months for FXR KO mice to develop liver cancer, we will introduce Met/β-catenin plasmids by using sleep beauty transposon system into FXR KO mice via hydrodynamic tail vein injection to induce liver cancer. In wild type mice, these plasmids, which have been provided by Dr. Xin Chen at UCSF, can induce liver cancer within a month post-injection [75, 76]. In the presence of dysbiosis and dysregulated BAs, liver cancer formation is expected to be facilitated and may occur even earlier. Using this model, we will study the effect of combined effect of retinoid plus HDAC inhibitors in liver cancer treatment.

Similar to Aim 1, we will test the combined effect of retinoid and SCFAs in liver cancer treatment. For example, if fenretinide plus butyrate gives the most robust apoptosis with the highest HDAC inhibitory effect, we will treat the mice using fenretinide plus 6% inulin (fructo-oligosaccharide). Inulin is a highly fermentable fiber that generates butyrate [77]. It has been shown this high fiber diet can increase butyrate and its HDAC inhibitory activity [78]. High fiber intake is a feasible dietary modification to shift gut microbiota and increase butyrate. In parallel, we will orally administer butyrate because this method will provide a clear dose effect for comparison. Moreover, our preliminary data already revealed the beneficial effect of butyrate delivered by oral gavage. Furthermore, it has been shown that oral delivery of ¹³C-label butyrate can reach the gut and alter histone modification [79].

### Methodology and Analyses for Aim 2

FXR KO mice in C57BL/6 background will receive hydrodynamic injection of Met/β-catenin plasmids by using sleep beauty transposon system when they are 2 months old using the published protocol [80]. Liver cancer formation will occur within a month. Mice will be divided into 6 groups (at least 12 mice per group) to receive vehicle control, fenretinide (60 mg/kg body weight [81]), 6% inulin, butyrate (5 g/kg body weight [82]), and combination of fenretinide plus inulin as well as fenretinide plus butyrate. All chemicals will be orally administered aiming to shift gut microbiota. The treatment will last for one month and mice will be euthanized for following assays. This experiment will be repeated for at least 2 times.

Morphological analysis: Tumor growth will be monitored by Positron Emission Tomography (PET) using the Core Service provided by Center for Molecular and Genomic Imaging at UC Davis. We will record liver-to-body weight ratio. Additionally, tumor burden will be determined by serial section of the livers every 3 mm to grossly count the nodules. Liver morphological analysis will be done using hematoxylin and eosin staining to determine the tumor type, and immunostaining of Ki67 will be performed for monitoring cell proliferation.

Quantification of bacterial genes: We will quantify the abundance of cecal microbial genes. The studied genes include, but are not limited to, butyryl-coenzyme-A-CoA transferase (bcoA) and butyrate kinase (buk) for butyrigenesis, methylmalonyl-CoA decarboxylase α-subunit (mmdA), CoA-dependent propionaldehyde dehydrogenase (pduP), and lactoyl-CoA dehydratase subunit a (lcdA) for propionate, baiJ and baiCD for secondary BAs production, as well as dsrA [51, 71, 83-86]. Due to the duration of this project (9 months), we will save the cecal DNA and perform 16S rRNA gene pyrosequencing in the future.

All other endpoints proposed under Aim 1 will be performed. Those include monitoring the level of apoptosis of cancer cells, the receptors for RA and SCFAs, miR-22, NUR77, RARβ, and histone deacetylase activity, inflammatory cytokines and metabolism genes, etc. Additionally, using immunohistochemistry, we will study tumor markers that include the expression of c-MYC and β-CATENIN as well as SIRT1, HDAC1, HDAC4, ands CYCLIN A2, which are all highly expressed in human liver cancers.

Relationship with Future R&D for Aim 2

If data generated from Aim 1 show the combination effect of fenretinide plus propionate is more robust than that of fenretinide plus butyrate, we will use apple pectin rather than inulin in the animal study because fermentation of pectin predominantly generates propionate [87].

We expect that retinoid including fenretinide by itself can shift microbiota since animal study shows that fenretinide can improve insulin sensitivity just like RA [88, 89], In addition, our published data already revealed that treating mice with RA prior to liver resection results in a lean microbiota phenotype, improved metabolism as well as accelerated liver regeneration. RA-treated mice had a reduced ratio of Firmicutes to Bacteroidetes one day after liver resection. In addition, elevated butyrate-generating Lachnospiraceae was observed in RA-treated mice post liver resection [90]. Those exciting data suggested that the anti-cancer effect of RA in part may due to altering gut microbiota to improve metabolism and boost gut immunity. We anticipate that treating mice with fenretinide plus inulin will further enrich the abundance of butyrate-generating bacteria, thereby increasing HDAC inhibitory effect to kill the cancer cells. SCFAs are not expected to damage normal cells because they are natural chemicals found in human and mice.

If the anti-cancer effect is not robust, we propose the following alternative approaches. We will consider adding a low dose of SAHA as mentioned under Aim 1. We can also increase the amount of inulin or pectin or consider using both. The generated data will also provide a foundation for the Phase II R&D. For example, we can consider using nano-formulated fenretinide that targets liver specifically to further improve the efficacy of fenretinide in combination treatment.

### Potential Commercial Application

Out of all the matrices proposed to evaluate success, we consider experimental success if a combined treatment using SCFAs plus fenretinide can generate similar HDAC inhibitory effect as SAHA alone since SAHA is very toxic. Alternatively, we will consider using synbiotic approaches i.e. treat mice with inulin plus butyrate-generating Roseburia hominis that is FDA approved or apple pectin plus propionate-generating Propionibacterium freudenreichii, which is also FDA GRAS approved [91-93]. Those experiments can also be considered in a future Phase II study. Because we only use natural chemicals or FDA-approved reagents, the success of this project should have immediate commercial values.

### REFERENCES

[1] Bukhari M H, Qureshi S S, Niazi S, Asef M, Naheed M, Khan S A, Chaudhry N A, Tayyab M, Hasan M. Chemotherapeutic/chemopreventive role of retinoids in chemically induced skin carcinogenesis in albino mice. Int J Dermatol. 2007; 46:1160-5.
[2] Wang Y, Wen W, Yi Y, Zhang Z, Lubet R A, You M. Preventive effects of bexarotene and budesonide in a genetically engineered mouse model of small cell lung cancer. Cancer Prev Res (Phila). 2009; 2:1059-64.
[3] Pisano C, Vesci L, Fodera R, Ferrara F F, Rossi C, De Cesare M, Zuco V, Pratesi G, Supino R, Zunino F. Antitumor activity of the combination of synthetic retinoid ST1926 and cisplatin in ovarian carcinoma models. Ann Oncol. 2007; 18:1500-5.
[4] Shah R K, Valdez T A, Wang Z, Shapshay S M. Pulsed-dye laser and retinoic acid delay progression of oral squamous cell carcinoma: a murine model. Laryngoscope. 2001; 111:1203-8.
[5] Li Y, Zhang Y, Hill J, Kim H T, Shen Q, Bissonnette R P, Lamph W W, Brown P H. The rexinoid, bexarotene, prevents the development of premalignant lesions in MMTV-erbB2 mice. Br J Cancer. 2008; 98:1380-8.
[6] Moon R C, Kelloff G J, Detrisac C J, Steele V E, Thomas C F, Sigman C C. Chemoprevention of OH-BBN-induced bladder cancer in mice by oltipraz, alone and in combination with 4-HPR and DFMO. Anticancer Res. 1994; 14:5-11.
[7] Liu B, Lee K W, Li H, Ma L, Lin G L, Chandraratna R A, Cohen P. Combination therapy of insulin-like growth factor binding protein-3 and retinoid X receptor ligands synergize on prostate cancer cell apoptosis in vitro and in vivo. Clin Cancer Res. 2005; 11:4851-6.
[8] Muto Y, Moriwaki H, Shiratori Y. Prevention of second primary tumors by an acyclic retinoid, polyprenoic acid, in patients with hepatocellular carcinoma. Digestion. 1998; 59 Suppl 2:89-91.
[9] Recchia F, Sica G, Candeloro G, Necozione S, Bisegna R, Bratta M, Rea S. Beta-interferon, retinoids and tamoxifen in metastatic breast cancer: long-term follow-up of a phase II study. Oncol Rep. 2009; 21:1011-6.
[10] Edelman M J, Smith R, Hausner P, Doyle L A, Kalra K, Kendall J, Bedor M, Bisaccia S. Phase II trial of the novel retinoid, bexarotene, and gemcitabine plus carboplatin in advanced non-small-cell lung cancer. J Clin Oncol. 2005; 23:5774-8.
[11] Yamane A, Tsukamoto N, Saitoh T, Uchiumi H, Handa H, Karasawa M, Nojima Y, Murakami H. Successful treatment by all-trans retinoic acid in a patient with acute promyelocytic leukemia complicated by liver cirrhosis and polycystic kidney. Intern Med. 2009; 48:1691-4.
[12] Colombo N, Formelli F, Cantu M G, Parma G, Gasco M, Argusti A, Santinelli A, Montironi R, Cavadini E, Baglietto L, Guerrieri-Gonzaga A, Viale G, Decensi A. A phase I-II preoperative biomarker trial of fenretinide in ascitic ovarian cancer. Cancer Epidemiol Biomarkers Prev. 2006; 15:1914-9.
[13] Hail N, Jr., Kim H J, Lotan R. Mechanisms of fenretinide-induced apoptosis. Apoptosis. 2006; 11:1677-94.
[14] Paulson J D, Oldham J W, Preston R F, Newman D. Lack of genotoxicity of the cancer chemopreventive agent N-(4-hydroxyphenyl)retinamide. Fundam Appl Toxicol. 1985; 5:144-50.
[15] Schneider B J, Worden F P, Gadgeel S M, Parchment R E, Hodges C M, Zwiebel J, Dunn R L, Wozniak A J, Kraut M J, Kalemkerian G P. Phase II trial of fenretinide (NSC 374551) in patients with recurrent small cell lung cancer. Invest New Drugs. 2009; 27:571-8.
[16] Evans R M, Mangelsdorf D J. Nuclear Receptors, RXR, and the Big Bang. Cell. 2014; 157:255-66.
[17] Bushue N, Wan Y J. Retinoid pathway and cancer therapeutics. Adv Drug Deliv Rev. 2010; 62:1285-98.
[18] Compare D, Coccoli P, Rocco A, Nardone O M, De Maria S, Carteni M, Nardone G. Gut—liver axis: the impact of gut microbiota on non alcoholic fatty liver disease. Nutr Metab Cardiovasc Dis. 2012; 22:471-6.
[19] Bultman S J. The microbiome and its potential as a cancer preventive intervention. Semin Oncol. 2016; 43:97-106.
[20] Manal M, Chandrasekar M J, Gomathi Priya J, Nanjan M J. Inhibitors of histone deacetylase as antitumor agents: A critical review. Bioorg Chem. 2016; 67:18-42.
[21] Canfora E E, Jocken J W, Blaak E E. Short-chain fatty acids in control of body weight and insulin sensitivity. Nat Rev Endocrinol. 2015; 11:577-91.
[22] Khan S, Jena G. The role of butyrate, a histone deacetylase inhibitor in diabetes mellitus: experimental evidence for therapeutic intervention. Epigenomics. 2015; 7:669-80.
[23] Vrieze A, Van Nood E, Holleman F, Salojarvi J, Kootte R S, Bartelsman J F, Dallinga-Thie G M, Ackermans M T, Serlie M J, Oozeer R, Derrien M, Druesne A, Van Hylckama Vlieg J E, Bloks V W, Groen A K, Heilig H G, Zoetendal E G, Stroes E S, de Vos W M, Hoekstra JB, Nieuwdorp M. Transfer of intestinal microbiota from lean donors increases insulin sensitivity in individuals with metabolic syndrome. Gastroenterology. 2012; 143:913-6 e7.
[24] Karlsson F H, Tremaroli V, Nookaew I, Bergstrom G, Behre C J, Fagerberg B, Nielsen J, Backhed F. Gut metagenome in European women with normal, impaired and diabetic glucose control. Nature. 2013; 498:99-103.
[25] Qin J, Li Y, Cai Z, Li S, Zhu J, Zhang F, Liang S, Zhang W, Guan Y, Shen D, Peng Y, Zhang D, Jie Z, Wu W, Qin Y, Xue W, Li J, Han L, Lu D, Wu P, Dai Y, Sun X, Li Z, Tang A, Zhong S, Li X, Chen W, Xu R, Wang M, Feng Q, Gong M, Yu J, Zhang Y, Zhang M, Hansen T, Sanchez G, Raes J, Falony G, Okuda S, Almeida M, LeChatelier E, Renault P, Pons N, Batto J M, Zhang Z, Chen H, Yang R, Zheng W, Li S, Yang H, Wang J, Ehrlich S D, Nielsen R, Pedersen O, Kristiansen K, Wang J. A metagenome-wide association study of gut microbiota in type 2 diabetes. Nature. 2012; 490:55-60.
[26] Vrieze A, Out C, Fuentes S, Jonker L, Reuling I, Kootte R S, van Nood E, Holleman F, Knaapen M, Romijn J A, Soeters M R, Blaak E E, Dallinga-Thie G M, Reijnders D, Ackermans M T, Serlie M J, Knop F K, Holst J J, van der Ley C, Kema I P, Zoetendal E G, de Vos W M, Hoekstra J B, Stroes E S, Groen A K, Nieuwdorp M. Impact of oral vancomycin on gut microbiota, bile acid metabolism, and insulin sensitivity. J Hepatol. 2014; 60:824-31.
[27] Wu N, Yang X, Zhang R, Li J, Xiao X, Hu Y, Chen Y, Yang F, Lu N, Wang Z, Luan C, Liu Y, Wang B, Xiang C, Wang Y, Zhao F, Gao G F, Wang S, Li L, Zhang H, Zhu B. Dysbiosis signature of fecal microbiota in colorectal cancer patients. Microb Ecol. 2013; 66:462-70.
[28] Marchesi J R, Adams D H, Fava F, Hermes G D, Hirschfield G M, Hold G, Quraishi M N, Kinross J, Smidt H, Tuohy K M, Thomas L V, Zoetendal E G, Hart A. The gut microbiota and host health: a new clinical frontier. Gut. 2016; 65:330-9.
[29] Rossen N G, Fuentes S, Boonstra K, D'Haens G R, Heilig H G, Zoetendal E G, de Vos W M, Ponsioen C Y. The mucosa-associated microbiota of PSC patients is characterized by low diversity and low abundance of uncultured Clostridiales I I. J Crohns Colitis. 2015; 9:342-8.
[30] Qin N, Yang F, Li A, Prifti E, Chen Y, Shao L, Guo J, Le Chatelier E, Yao J, Wu L, Zhou J, Ni S, Liu L, Pons N, Batto J M, Kennedy S P, Leonard P, Yuan C, Ding W, Chen Y, Hu X, Zheng B, Qian G, Xu W, Ehrlich S D, Zheng S, Li L. Alterations of the human gut microbiome in liver cirrhosis. Nature. 2014; 513:59-64.
[31] Singh N, Gurav A, Sivaprakasam S, Brady E, Padia R, Shi H, Thangaraju M, Prasad P D, Manicassamy S, Munn D H, Lee J R, Offermanns S, Ganapathy V. Activation of Gpr109a, receptor for niacin and the commensal metabolite butyrate, suppresses colonic inflammation and carcinogenesis. Immunity. 2014; 40:128-39.
[32] Iwata M, Hirakiyama A, Eshima Y, Kagechika H, Kato C, Song S Y. Retinoic acid imprints gut-homing specificity on T cells. Immunity. 2004; 21:527-38.
[33] Julia V, Macia L, Dombrowicz D. The impact of diet on asthma and allergic diseases. Nat Rev Immunol. 2015; 15:308-22.
[34] Pages F, Berger A, Lebel-Binay S, Zinzindohoue F, Danel C, Piqueras B, Carriere O, Thiounn N, Cugnenc P H, Fridman W H. Proinflammatory and antitumor properties of interleukin-18 in the gastrointestinal tract. Immunol Lett. 2000; 75:9-14.
[35] Yang F, Hu Y, Liu H X, Wan Y J. MiR-22-silenced cyclin A expression in colon and liver cancer cells is regulated by bile acid receptor. J Biol Chem. 2015; 290:6507-15.
[36] Mudaliar S, Henry R R, Sanyal A J, Morrow L, Marschall H U, Kipnes M, Adorini L, Sciacca C I, Clopton P, Castelloe E, Dillon P, Pruzanski M, Shapiro D. Efficacy and safety of the farnesoid X receptor agonist obeticholic acid in patients with type 2 diabetes and nonalcoholic fatty liver disease. Gastroenterology. 2013; 145:574-82 el.
[37] Kim I, Morimura K, Shah Y, Yang Q, Ward J M, Gonzalez F J. Spontaneous hepatocarcinogenesis in farnesoid X receptor-null mice. Carcinogenesis. 2007; 28:940-6.
[38] Yang F, Huang X, Yi T, Yen Y, Moore D D, Huang W. Spontaneous development of liver tumors in the absence of the bile acid receptor farnesoid X receptor. Cancer Res. 2007; 67:863-7.
[39] Su H, Ma C, Liu J, Li N, Gao M, Huang A, Wang X, Huang W, Huang X. Downregulation of nuclear receptor FXR is associated with multiple malignant clinicopathological characteristics in human hepatocellular carcinoma. Am J Physiol Gastrointest Liver Physiol. 2012; 303:G1245-53.
[40] Maran R R, Thomas A, Roth M, Sheng Z, Esterly N, Pinson D, Gao X, Zhang Y, Ganapathy V, Gonzalez F J, Guo G L. Farnesoid X receptor deficiency in mice leads to increased intestinal epithelial cell proliferation and tumor development. J Pharmacol Exp Ther. 2009; 328:469-77.
[41] Modica S, Murzilli S, Salvatore L, Schmidt D R, Moschetta A. Nuclear bile acid receptor FXR protects against intestinal tumorigenesis. Cancer Res. 2008; 68:9589-94.
[42] Ling B, Wang G X, Long G, Qiu J H, Hu Z L. Tumor suppressor miR-22 suppresses lung cancer cell progression through post-transcriptional regulation of ErbB3. J Cancer Res Clin Oncol. 2012; 138:1355-61.
[43] Xiong J, Yu D, Wei N, Fu H, Cai T, Huang Y, Wu C, Zheng X, Du Q, Lin D, Liang Z. An estrogen receptor alpha suppressor, microRNA-22, is downregulated in estrogen receptor alpha-positive human breast cancer cell lines and clinical samples. FEBS J. 2010; 277:1684-94.
[44] Yamakuchi M, Yagi S, Ito T, Lowenstein C J. MicroRNA-22 regulates hypoxia signaling in colon cancer cells. PLoS One. 2011; 6:e20291.
[45] Tsuchiya N, Izumiya M, Ogata-Kawata H, Okamoto K, Fujiwara Y, Nakai M, Okabe A, Schetter A J, Bowman E D, Midorikawa Y, Sugiyama Y, Aburatani H, Harris C C, Nakagama H. Tumor suppressor miR-22 determines p53-dependent cellular fate through post-transcriptional regulation of p21. Cancer Res. 2011; 71:4628-39.
[46] Zhang J, Yang Y, Yang T, Liu Y, Li A, Fu S, Wu M, Pan Z, Zhou W. microRNA-22, downregulated in hepatocellular carcinoma and correlated with prognosis, suppresses cell proliferation and tumourigenicity. Br J Cancer. 2010; 103:1215-20.
[47] Xiong J, Du Q, Liang Z. Tumor-suppressive microRNA-22 inhibits the transcription of E-box-containing c-Myc target genes by silencing c-Myc binding protein. Oncogene. 2010; 29:4980-8.
[48] Xu D, Takeshita F, Hino Y, Fukunaga S, Kudo Y, Tamaki A, Matsunaga J, Takahashi R U, Takata T, Shimamoto A, Ochiya T, Tahara H. miR-22 represses cancer progression by inducing cellular senescence. J Cell Biol. 2011; 193:409-24.
[49] Li J, Zhang Y, Zhao J, Kong F, Chen Y. Overexpression of miR-22 reverses paclitaxel-induced chemoresistance through activation of PTEN signaling in p53-mutated colon cancer cells. Mol Cell Biochem. 2011; 357:31-8.
[50] Kitahara M, Sakata S, Sakamoto M, Benno Y. Comparison among fecal secondary bile acid levels, fecal microbiota and Clostridium scindens cell numbers in Japanese. Microbiol Immunol. 2004; 48:367-75.
[51] Yoshimoto S, Loo T M, Atarashi K, Kanda H, Sato S, Oyadomari S, Iwakura Y, Oshima K, Morita H, Hattori M, Honda K, Ishikawa Y, Hara E, Ohtani N. Obesity-induced gut microbial metabolite promotes liver cancer through senescence secretome. Nature. 2013; 499:97-101.
[52] Wu H, Lin Y, Li W, Sun Z, Gao W, Zhang H, Xie L, Jiang F, Qin B, Yan T, Chen L, Zhao Y, Cao X, Wu Y, Lin B, Zhou H, Wong A S, Zhang X K, Zeng J Z. Regulation of Nur77 expression by beta-catenin and its mitogenic effect in colon cancer cells. FASEB J. 2011; 25:192-205.
[53] Moll U M, Marchenko N, Zhang X K. p53 and Nur77/TR3—transcription factors that directly target mitochondria for cell death induction. Oncogene. 2006; 25:4725-43.
[54] Hu Y, Chau T, Liu H X, Liao D, Keane R, Nie Y, Yang H, Wan Y J. Bile acids regulate nuclear receptor (Nur77) expression and intracellular location to control proliferation and apoptosis. Mol Cancer Res. 2015; 13:281-92.
[55] Safe S, Jin U H, Hedrick E, Reeder A, Lee S O. Minireview: role of orphan nuclear receptors in cancer and potential as drug targets. Mol Endocrinol. 2014; 28:157-72.
[56] Lee S O, Jin U H, Kang J H, Kim S B, Guthrie A S, Sreevalsan S, Lee J S, Safe S. The orphan nuclear receptor NR4A1 (Nur77) regulates oxidative and endoplasmic reticulum stress in pancreatic cancer cells. Mol Cancer Res. 2014; 12:527-38.
[57] Martinez-Gonzalez J, Badimon L. The NR4A subfamily of nuclear receptors: new early genes regulated by growth factors in vascular cells. Cardiovasc Res. 2005; 65:609-18.
[58] Lin B, Kolluri S K, Lin F, Liu W, Han Y H, Cao X, Dawson M I, Reed J C, Zhang X K. Conversion of Bcl-2 from protector to killer by interaction with nuclear orphan receptor Nur77/TR3. Cell. 2004; 116:527-40.
[59] Yang H, Zhan Q, Wan Y J. Enrichment of Nur77 mediated by retinoic acid receptor beta leads to apoptosis of human hepatocellular carcinoma cells induced by fenretinide and histone deacetylase inhibitors. Hepatology. 2011; 53:865-74.
[60] Yang H, Nie Y, Li Y, Wan Y J. ERK1/2 deactivation enhances cytoplasmic Nur77 expression level and improves the apoptotic effect of fenretinide in human liver cancer cells. Biochem Pharmacol. 2011; 81:910-6.
[61] De Ruyter M G, Lambert W E, De Leenheer A P. Retinoic acid: an endogenous compound of human blood. Unequivocal demonstration of endogenous retinoic acid in normal physiological conditions. Anal Biochem. 1979; 98:402-9.
[62] Muindi J R, Frankel S R, Huselton C, DeGrazia F, Garland W A, Young C W, Warrell R P, Jr. Clinical pharmacology of oral all-trans retinoic acid in patients with acute promyelocytic leukemia. Cancer Res. 1992; 52:2138-42.
[63] Suzuki T, Yoshida S, Hara H. Physiological concentrations of short-chain fatty acids immediately suppress colonic epithelial permeability. Br J Nutr. 2008; 100:297-305.
[64] Cummings J H, Pomare E W, Branch W J, Naylor C P, Macfarlane G T. Short chain fatty acids in human large intestine, portal, hepatic and venous blood. Gut. 1987; 28:1221-7.
[65] Hu Y, Liu H X, He Y, Fang Y, Fang J, Wan Y J. Transcriptome profiling and genome-wide DNA binding define the differential role of fenretinide and all-trans RA in regulating the death and survival of human hepatocellular carcinoma Huh? cells. Biochem Pharmacol. 2013; 85:1007-17.
[66] Bloemen J G, Venema K, van de Poll M C, Olde Damink S W, Buurman W A, Dejong C H. Short chain fatty acids exchange across the gut and liver in humans measured at surgery. Clin Nutr. 2009; 28:657-61.
[67] den Besten G, van Eunen K, Groen A K, Venema K, Reijngoud D J, Bakker B M. The role of short-chain fatty acids in the interplay between diet, gut microbiota, and host energy metabolism. J Lipid Res. 2013; 54:2325-40.
[68] Tsuei J, Chau T, Mills D, Wan Y J. Bile acid dysregulation, gut dysbiosis, and gastrointestinal cancer. Exp Biol Med (Maywood). 2014; 239:1489-504.
[69] Liu N, Meng Z, Lou G, Zhou W, Wang X, Zhang Y, Zhang L, Liu X, Yen Y, Lai L, Forman B M, Xu Z, Xu R, Huang W. Hepatocarcinogenesis in FXR−/− mice mimics human HCC progression that operates through HNF1alpha regulation of FXR expression. Mol Endocrinol. 2012; 26:775-85.
[70] Attene-Ramos M S, Wagner E D, Plewa M J, Gaskins H R. Evidence that hydrogen sulfide is a genotoxic agent. Mol Cancer Res. 2006; 4:9-14.
[71] Ijssennagger N, Belzer C, Hooiveld G J, Dekker J, van Mil S W, Muller M, Kleerebezem M, van der Meer R. Gut microbiota facilitates dietary heme-induced epithelial hyperproliferation by opening the mucus barrier in colon. Proc Natl Acad Sci USA. 2015; 112:10038-43.
[72] Martinez I, Perdicaro D J, Brown A W, Hammons S, Carden T J, Carr T P, Eskridge K M, Walter J. Diet-induced alterations of host cholesterol metabolism are likely to affect the gut microbiota composition in hamsters. Appl Environ Microbiol. 2013; 79:516-24.
[73] Ridlon J M, Kang D J, Hylemon P B. Bile salt biotransformations by human intestinal bacteria. J Lipid Res. 2006; 47:241-59.
[74] Lee C G, Kim Y W, Kim E H, Meng Z, Huang W, Hwang S J, Kim S G. Farnesoid X receptor protects hepatocytes from injury by repressing miR-199a-3p, which increases levels of LKB1. Gastroenterology. 2012; 142:1206-17 e7.
[75] Tward A D, Jones K D, Yant S, Cheung S T, Fan S T, Chen X, Kay M A, Wang R, Bishop J M. Distinct pathways of genomic progression to benign and malignant tumors of the liver. Proc Natl Acad Sci USA. 2007; 104:14771-6.
[76] Chen X, Calvisi D F. Hydrodynamic transfection for generation of novel mouse models for liver cancer research. Am J Pathol. 2014; 184:912-23.
[77] Donohoe D R, Holley D, Collins L B, Montgomery S A, Whitmore A C, Hillhouse A, Curry K P, Renner S W, Greenwalt A, Ryan E P, Godfrey V, Heise M T, Threadgill D S, Han A, Swenberg J A, Threadgill D W, Bultman S J. A gnotobiotic mouse model demonstrates that dietary fiber protects against colorectal tumorigenesis in a microbiota- and butyrate-dependent manner. Cancer Discov. 2014; 4:1387-97.
[78] Zeng H, Lazarova D L, Bordonaro M. Mechanisms linking dietary fiber, gut microbiota and colon cancer prevention. World J Gastrointest Oncol. 2014; 6:41-51.
[79] Mathewson N D, Jenq R, Mathew A V, Koenigsknecht M, Hanash A, Toubai T, Oravecz-Wilson K, Wu S R, Sun Y, Rossi C, Fujiwara H, Byun J, Shono Y, Lindemans C, Calafiore M, Schmidt T C, Honda K, Young V B, Pennathur S, van den Brink M, Reddy P. Gut microbiome-derived metabolites modulate intestinal epithelial cell damage and mitigate graft-versus-host disease. Nat Immunol. 2016; 17:505-13.
[80] Bell J B, Podetz-Pedersen K M, Aronovich E L, Belur L R, McIvor R S, Hackett P B. Preferential delivery of the Sleeping Beauty transposon system to livers of mice by hydrodynamic injection. Nat Protoc. 2007; 2:3153-65.
[81] Kanagaratham C, Kalivodova A, Najdekr L, Friedecky D, Adam T, Hajduch M, De Sanctis J B, Radzioch D. Fenretinide prevents inflammation and airway hyperresponsiveness in a mouse model of allergic asthma. Am J Respir Cell Mol Biol. 2014; 51:783-92.
[82] Gao Z, Yin J, Zhang J, Ward R E, Martin R J, Lefevre M, Cefalu W T, Ye J. Butyrate improves insulin sensitivity and increases energy expenditure in mice. Diabetes. 2009; 58:1509-17.
[83] Louis P, Flint H J. Development of a semiquantitative degenerate real-time per-based assay for estimation of numbers of butyryl-coenzyme A (CoA) CoA transferase genes in complex bacterial samples. Appl Environ Microbiol. 2007; 73:2009-12.
[84] O'Keefe S J, Li J V, Lahti L, Ou J, Carbonero F, Mohammed K, Posma J M, Kinross J, Wahl E, Ruder E, Vipperla K, Naidoo V, Mtshali L, Tims S, Puylaert P G, DeLany J, Krasinskas A, Benefiel A C, Kaseb H O, Newton K, Nicholson J K, de Vos W M, Gaskins H R, Zoetendal E G. Fat, fibre and cancer risk in African Americans and rural Africans. Nat Commun. 2015; 6:6342.
[85] Ou J, Carbonero F, Zoetendal E G, DeLany J P, Wang M, Newton K, Gaskins H R, O'Keefe S J. Diet, microbiota, and microbial metabolites in colon cancer risk in rural Africans and African Americans. Am J Clin Nutr. 2013; 98:111-20.
[86] Vital M, Penton C R, Wang Q, Young V B, Antonopoulos D A, Sogin M L, Morrison H G, Raffals L, Chang E B, Huffnagle G B, Schmidt T M, Cole J R, Tiedje J M. A gene-targeted approach to investigate the intestinal butyrate-producing bacterial community. Microbiome. 2013; 1:8.
[87] Henningsson A M, Nyman E M, Bjorck I M. Content of short-chain fatty acids in the hindgut of rats fed processed bean (Phaseolus vulgaris) flours varying in distribution and content of indigestible carbohydrates. Br J Nutr. 2001; 86:379-89.
[88] Bikman B T, Guan Y, Shui G, Siddique M M, Holland W L, Kim J Y, Fabrias G, Wenk M R, Summers S A. Fenretinide prevents lipid-induced insulin resistance by blocking ceramide biosynthesis. J Biol Chem. 2012; 287:17426-37.
[89] Johansson H, Gandini S, Guerrieri-Gonzaga A, lodice S, Ruscica M, Bonanni B, Gulisano M, Magni P, Formelli F, Decensi A. Effect of fenretinide and low-dose tamoxifen on insulin sensitivity in premenopausal women at high risk for breast cancer. Cancer Res. 2008; 68:9512-8.
[90] Liu H X, Hu Y, Wan Y J. Microbiota and bile acid profiles in retinoic acid-primed mice that exhibit accelerated liver regeneration. Oncotarget. 2016; 7:1096-106.
[91] Travis A J, Kelly D, Flint H J, Aminov R I. Complete Genome Sequence of the Human Gut Symbiont Roseburia hominis. Genome Announc. 2015; 3.
[92] Ye M B, Lim B O. Dietary pectin regulates the levels of inflammatory cytokines and immunoglobulins in interleukin-10 knockout mice. J Agric Food Chem. 2010; 58:11281-6.
[93] Gueimonde M, Frias R, Ouwehand A C. Assuring the continued safety of lactic acid bacteria used as probiotics. Biologia. 2006; 61:755-60.
[94] Faul F, Erdfelder E, Lang A G, Buchner A. G*Power 3: a flexible statistical power analysis program for the social, behavioral, and biomedical sciences. Behav Res Methods. 2007; 39:175-91.

### Example 5. miR-22 Induced by Short-Chain Fatty Acids and Retinoid Acid Combats Colon Cancer Through HDAC Silencing and Epigenetic Regulation of NUR77 and RARβ

### Abstract

Fiber and β-carotene-enriched Mediterranean diet are considered healthy. This study investigated the interaction between retinoic acid (RA) and short-chain fatty acids (SCFAs) that have histone deacetylase (HDAC) inhibitory property. RA, SCFA, miR-22, and protein deacetylation signaling pathways were studied in human colon polyps and cancer specimens. The effects of RA and HDAC inhibitors on miR-22 expression as well as its downstream effects were analyzed in cells and mouse models. miR-22 and the receptors for RA and SCFAs were consistently reduced in colon polyps and cancers. Additionally, the abundance of bacterial butyrate-producing genes bcoA and buk was reduced, which was accompanied by elevated HDAC1, HDAC4, and SIRT1 in colon cancers. Luciferase reporter and ChIP-qPCR assays revealed that RA and HDAC inhibitors induced miR-22 via RARβ, and HDAC1 as a novel miR-22 target. Moreover, in a miR-22-dependent manner, butyrate and RA silenced HDAC1, HDAC4, and SIRT1, which were involved in chromatin remodeling of the NUR77 and RARβ genes and acetylation of those nuclear receptor proteins. Thus, through miR-22 induction and HDACs reduction, RA and butyrate promoted expression and nuclear export of NUR77 and RARβ to induce apoptosis. Furthermore, miR-22 and its inducers RA/butyrate inhibited colon cancer growth in xenograft mice. Moreover, treated tumors had elevated miR-22, NUR77, and RARβ as well as reduced HDACs. In conclusion, SCFAs plus RA-induced miR-22, which silences protein deacetylases to induce cytosolic NUR77 and RARβ, is an attractive therapeutic approach for colon cancer. This mechanism explains the beneficial effects of SCFAs and RA.

### Summary

Reduced miR-22 and impaired SCFA, RA, and protein deacetylase signaling are found in human polyps and colon cancer specimens.

miR-22 can be induced by RA as well as HDAC inhibitory SCFAs including butyrate, propionate, and valerate. In addition, a combination of RA plus HDAC inhibitor yielded greater miR-22 induction than a single agent.

HDAC1 is identified as a novel miR-22 target and miR-22 also inhibits HDAC1, HDAC4 and SIRT1 in colon cancer cells.

Through silencing of multiple HDACs, miR-22 epigenetically modifies nuclear receptors NUR77 and RARβ nuclear export, which cause colon cancer apoptosis.

HDAC inhibitor plus RA-induced miR-22 silencing of HDACs and upregulation of cytosolic NUR77/RARβ appears a novel therapeutic approach for colon cancer prevention and treatment.

### Introduction

MicroRNA-22 (miR-22) is highly conserved across vertebrate species, including chimp, mouse, rat, dog, and horse, suggesting its functional necessity. Additionally, miR-22 is ubiquitously expressed in various tissues and its protective effect against cancer has been demonstrated in various tissues and models 1, 2, 3, 4, 5, 6, 7. miR-22 is reduced in breast carcinoma, multiple myeloma, lung and colon as well as liver cancer^{2, 3, 6, 8}. In breast cancer, miR-22 restricts cell proliferation by targeting estrogen receptor α (ERα) and c-Myc-binding protein (MYCBP)^{4, 6}. In lung cancer, the tumor suppressive effect of miR-22 is associated with post-transcriptional regulation of Erb-B2 receptor tyrosine kinase 3 (ErbB3)⁷. In colon cancer, miR-22 inhibits tumorigenic hypoxia-inducible factor 1α (HIF-1α), vascular endothelial growth factor (VEGF), and P21 while enhancing paclitaxel-induced apoptosis in wild-type p53 colon cancer cells^{1, 5}. Moreover, bile acids induce miR-22 to silence CYCLIN A2 (CCNA2) and inhibit colon and liver cancer cell proliferation in a farnesoid X receptor (FXR)-dependent manner². Considering the beneficial effects of miR-22, it is crucial to elucidate the regulatory mechanism of miR-22 expression.

The Mediterranean diet, rich in fiber and β-carotene, has gained increasing recognition for its extensive health benefits. β-carotene is converted into vitamin A (retinol) which is then oxidized into retinoic acid (RA) to exert its biologic effects^{9, 10} Short-chain fatty acids (SCFAs) are generated from bacterial fermentation of indigestible dietary fiber. Among them, butyrate, propionate, and valerate have histone deacetylase (HDAC) inhibitory property that can combat cancer ^{11, 12, 13} ENREF 15. Butyrate is mainly utilized by colonocytes as an energy source ¹⁴, whereas, propionate is used for hepatic gluconeogenesis ¹⁵. In addition, butyrate also induces expression of tight junction proteins to maintain intestinal epithelium integrity. Moreover, butyrate increases expression of RA-producing enzyme aldehyde dehydrogenase 1A1 (ALDH1A1) in intestinal dendritic cells to enhance RA signaling¹⁶. Therefore, there appears to be a co-regulatory interaction between butyrate and RA.

The anti-carcinogenic effect of RA has traditionally been attributed to its anti-oxidant function while its other health benefits are less well-studied^{17, 18}. In a normal liver, RA is a mild mitogen that induces cell cycle genes to facilitate liver regeneration ^{19, 20} In cancer cells, RA is a weak apoptosis inducer²¹. However, a combination of retinoid plus HDAC inhibitor produces a potent pro-apoptotic effect^{22, 23, 24, 25, 26.} Similar to RA, orphan nuclear receptor NUR77 also has a dual role in regulating cell survival and death^{27, 28}. NUR77 transcriptional activity can be stimulated by mitogens such as EGF to increase the expression of proliferative cell cycle genes and anti-apoptotic genes²⁹. NUR77 is overexpressed in multiple cancer types including colon, liver, and pancreatic cancers, making NUR77 an attractive target for cancer treatment^{28, 30}. It has been shown that inactivation of NUR77 by NUR77-specific inhibitor DIM-C-pPhOH resulting in activation of ROS/ER stress and pro-apoptotic pathways represents a potential therapeutic strategy for pancreatic cancer³⁰. In contrast to oncogenic nuclear NUR77, cytosolic NUR77 is paradoxically pro-apoptotic³¹. Our published data revealed that retinoid plus HDAC inhibitor-induced apoptosis is cytosolic NUR77 and retinoic acid receptor β (RARβ)-dependent²².

Although their pro-apoptotic mechanism is not fully understood, HDAC inhibitors represent the most promising epigenetic cancer therapy due to their pleiotropic antitumor effects specifically targeting malignant cells³². The current study uncovered that miR-22 tumor suppressor can be induced by RA as well as HDAC inhibitory suberanilohydroxamic acid (SAHA) and SCFAs including butyrate, propionate, and valerate. In addition, a combination of RA plus HDAC inhibitor yielded greater miR-22 induction than a single agent. Moreover, our novel data revealed that miR-22 directly targeted HDAC1 and other protein deacetylases including HDAC4 and SIRT1, which were recruited to epigenetically modify the NUR77 and RARβ expression. Additionally, miR-22 and its inducers RA plus butyrate were effective in inhibiting the growth of colon cancer in athymic nude mice. Together, HDAC inhibitor plus RA-induced miR-22 silencing of protein deacetylases and upregulation of cytosolic NUR77/RARβ constitute a novel and attractive therapeutic approach for colon cancer.

### Results

Inverse Expression Pattern Between miR-22 and HDACs as Well as Impaired RA and SCFA Signaling in Human Colon Polyp and Cancer

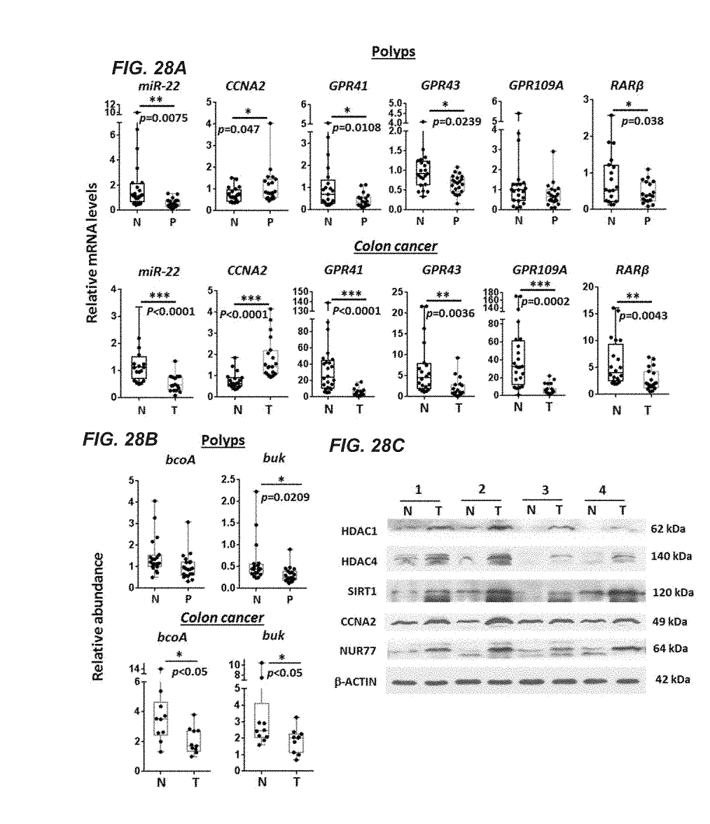

By screening various signaling pathways differentially expressed in normal versus colon polyps and cancer specimens, expression of SCFA receptors GPR41, 43, and 109A as well as RARβ, were reduced in disease specimens (FIG. 28A). Consistently, the abundance of bacterial genes bcoA and buk, which are responsible for butyrate production, were also reduced in colon polyp and cancer specimens (FIG. 28B)^{33, 34}. Moreover, the levels of miR-22 and CCNA2, a miR-22 target, were inversely correlated in normal versus polyp and cancer specimens (FIG. 28A). Furthermore, impaired miR-22 and SCFA signaling found in colon cancer specimens were accompanied by elevated levels of protein deacetylases HDAC1, HDAC4, and SIRT1 as well as CCNA2 and NUR77 (FIG. 28C). These findings suggest that SCFA and RA as well as miR-22 and HDAC signaling pathways are compromised during colon cancer carcinogenesis.

HDAC Inhibitors and RA Induce miR-22 Through Increased Recruitment of RARβ and RXRα to its Regulatory Region

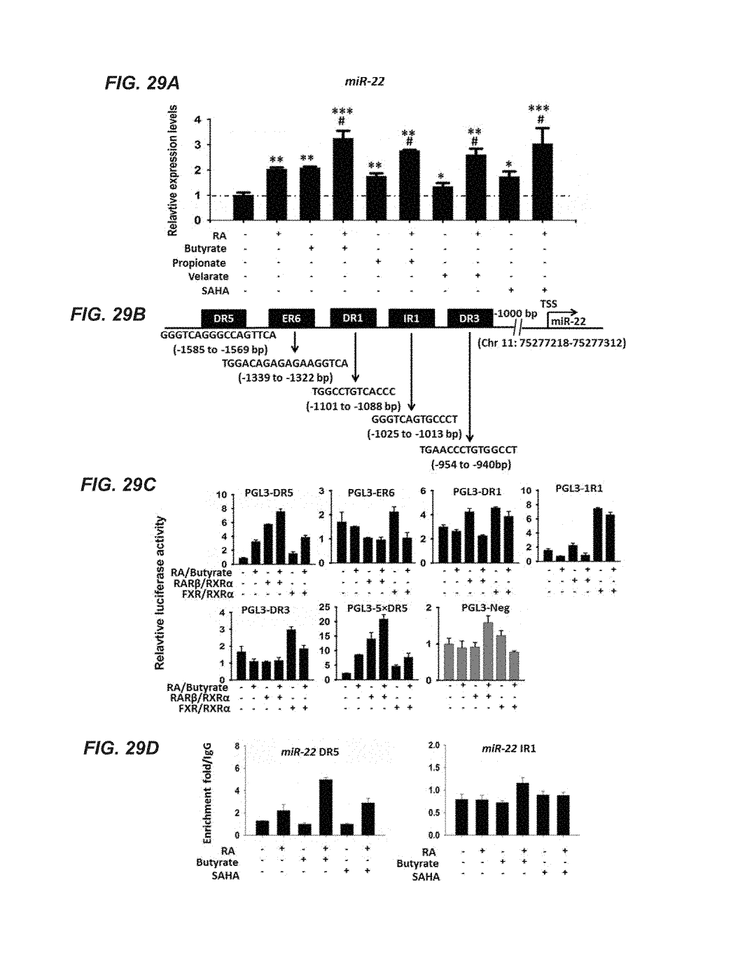

To study the effect of RA and HDAC inhibitors on miR-22 expression, HCT116 cells were treated with RA, butyrate, propionate, valerate, and SAHA. The SCFA concentrations used were within physiologic ranges³⁵. All agents increased miR-22 level within 24 hours. Additionally, combinations of RA plus HDAC inhibitors produced more robust miR-22 upregulation than the single agent (FIG. 29A). Moreover, RA/butyrate and RA/SAHA were equally effective in inducing miR-22. A combination of RA plus butyrate was used for subsequent studies.

By analyzing the sequence 2 Kb upstream of miR-22, multiple putative nuclear receptor binding motifs were identified (FIG. 29B). These motifs were cloned into the PGL-3 plasmid for transient transfection assays in HCT116 cells. PGL3-Neg and PGL3-5×DR5 were used as negative and positive controls, respectively. There was a 5-fold increase in luciferase activity after co-transfection with RARβ/RXRα expression plasmids along with PGL3-DR5 and a 7.5-fold increase with RA/butyrate treatment (FIG. 29C). In addition, co-transfection of PGL3-5×DR5, a positive control for RARβ, increased luciferase activity, indicating RARβ/RXRα binding effects. In comparison, co-transfection of PGL3-IR1 along with FXR/RXRα increased luciferase activity by 7-fold, but RA/butyrate did not further increase luciferase activity (FIG. 29C). In contrast, PGL3-Neg and other motifs including DR1, ER6, and DR3, responded to neither RARβ/RXRα overexpression nor RA/butyrate treatment (FIG. 29C). In addition, ChIP-qPCR data revealed that RA/butyrate as well as RA/SAHA increased the recruitment of RARβ to a DR-5, but did not change the binding of RARβ to an IR-1, a previously characterized FXR binding site (FIG. 29D)². Taken together, miR-22 induction by RA/butyrate was transcriptionally regulated by RARβ/RXRα binding to a DR-5 motif located in the miR-22 regulatory region.

miR-22 Silences Multiple Protein Deacetylases

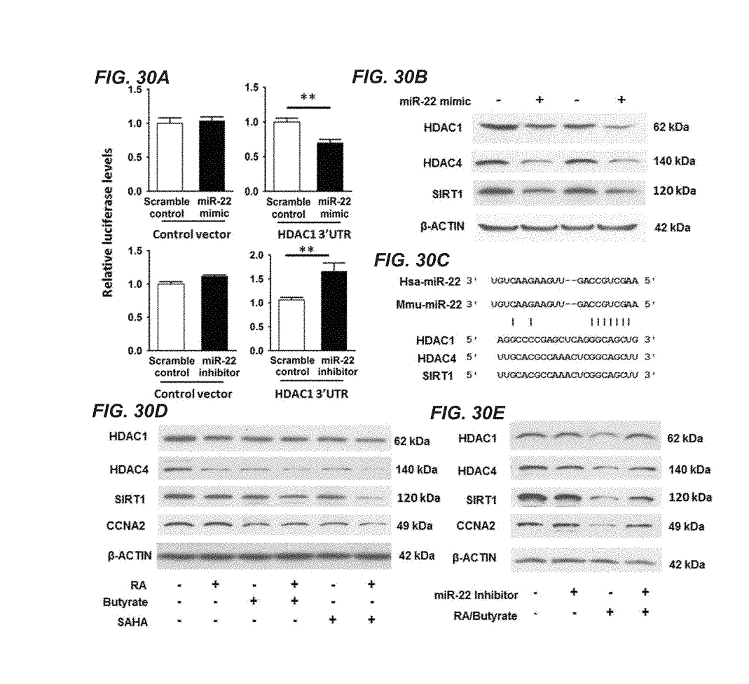

Sequence alignment shows that miR-22 partially pairs with the 3′UTR of HDAC1. To establish miR-22 inhibition of HDAC1, in vitro functional assays were conducted using miR-22 mimics and inhibitors. psiCHECK2-HDAC1 containing the 3′UTR of HDAC1 was constructed and co-transfected with either miR-22 mimics or inhibitors into HCT116 cells. Scramble mimics and inhibitors were included as controls. miR-22 mimics inhibited luciferase activity of psiCHECK2-HDAC1 by roughly 30%, while miR-22 inhibitors stimulated increased luciferase activity of psiCHECK2-HDAC1 (FIG. 30A). Furthermore, HDAC1 protein level was reduced by miR-22 transfection after 48 hours (FIG. 30B). In addition, miR-22 reduced HDAC4 and SIRT1 in HCT116 cells, consistent with the data shown in rat cardiomyocytes³⁶. Together, HDAC1 is a novel miR-22 direct target, and miR-22 inhibits multiple protein deacetylases in colon cancer cells

HDAC1, HDAC4, SIRT1, and CCNA2 Reduction by RA/Butyrate is miR-22 Dependent in Colon Cancer Cells

Consistent with miR-22 inhibition of HDACs, RA as well as butyrate and SAHA reduced HDAC1, HDAC4, SIRT1, and CCNA2 protein levels. In addition, a combination of RA plus HDAC inhibitor resulted in even greater reduction than single agent treatment (FIG. 30D). Furthermore, miR-22 inhibitors prevented RA/butyrate reduction of HDAC1, HDAC4, SIRT1, and CCNA2 protein levels in HCT116 cells (FIG. 30E). Together, RA plus HDAC inhibitors down-regulate HDACs and CCNA2 in a miR-22-dependent manner. Thus, miR-22-silenced HDACs and cell cycle arrest may serve as mechanisms by which butyrate/RA exert their anti-carcinogenic effect.

### RA/Butyrate Potently Promote Apoptosis by Inducing NUR77 and RARβ

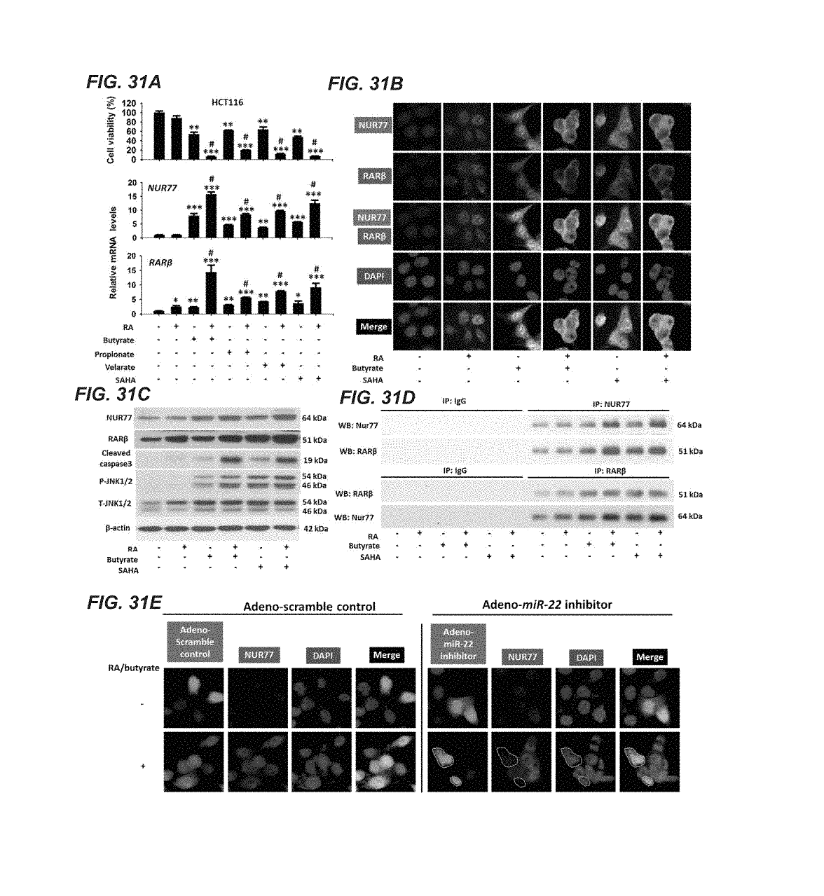

We previously reported that a combination of a synthetic retinoid plus HDAC inhibitors, i.e. scriptaid or trichostatin A induces apoptosis in a NUR77 and RARβ-dependent manner in liver cancer cells²². To determine the pro-apoptotic effect of RA in combination with HDAC inhibitors, HCT116 cells were treated with RA and/or butyrate, propionate, valerate, and SAHA. Markedly reduced cell viability was observed in all treatments except for RA alone. Moreover, combinations of RA plus HDAC inhibitors were more potent than single agent treatment (FIG. 31A). Greater than 80% of HCT116 cells died after 48 hours in all the combination treatment groups. Moreover, cell viability and NUR77 and RARβ mRNA levels were inversely correlated, supporting the involvement of these nuclear receptors (FIG. 31A).

Immunofluorescence staining data revealed that butyrate and SAHA markedly induced NUR77 and RARβ. Both receptors were diffusely found in both nucleus and cytosol whereas RA modestly induced both receptors in the nucleus. However, RA/butyrate or RA/SAHA induced greater cytosolic NUR77 and RARβ (FIG. 31B). Moreover, Western blot data revealed that induction of cytosolic NUR77 and RARβ by RA/butyrate or RA/SAHA was accompanied by increased cleaved caspase 3 and phosphorylated-JNK1/2 (FIG. 31C). Furthermore, immunoprecipitation followed by Western blot showed the interaction between NUR77 and RARβ (FIG. 31D). To prove that induction of cytosolic NUR77 by RA/butyrate is miR-22-mediated, adeno-miR-22 inhibitor-GFP were used to infect HCT116 cells. Immunostaining data revealed that miR-22 inhibitors prohibited NUR77 induction as well as nuclear export caused by RA/butyrate (FIG. 31E). Elevated NUR77 was only found in uninfected, GFP-negative cells (FIG. 31E). Taken together, miR-22 is essential for RA/butyrate to induce cytosolic NUR77 to enact apoptosis.

### RA and HDAC Inhibitors Epigenetically Regulate NUR77 and RARβ Expression

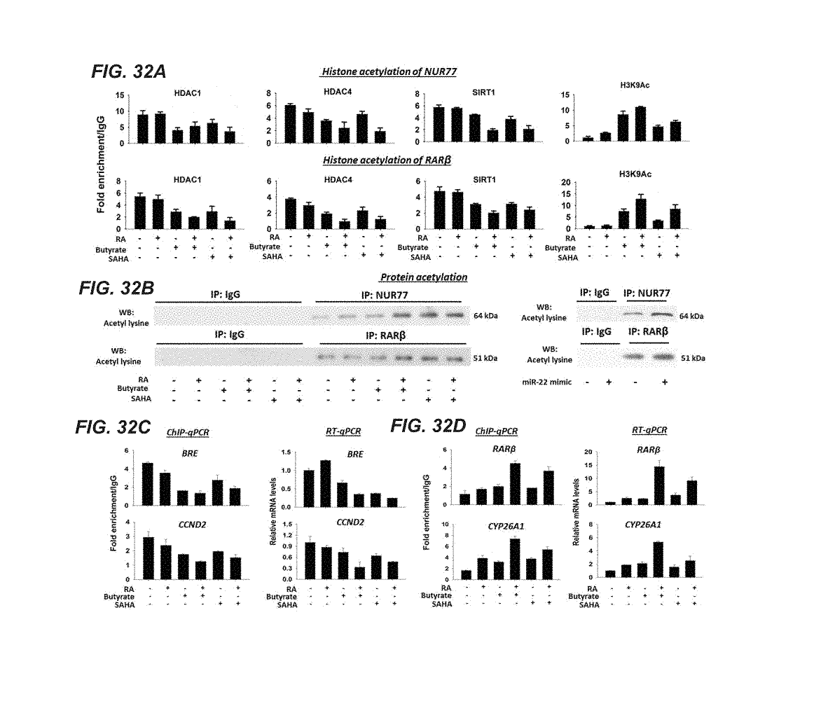

ChIP-qPCR was performed to assess the involvement of HDAC1, HDAC4, and SIRT1 in regulating NUR77 and RARβ expression. The data revealed that butyrate and SAHA, but not RA, decreased recruitment of HDAC1, HDAC4, and SIRT1 to the transcriptional regulatory region of NUR77 and RARβ in HCT116 cells (FIG. 32A). When RA was presented in combination with butyrate or SAHA, recruitment of each HDAC was further reduced. Moreover, butyrate, SAHA, and their combination with RA increased H3K9 acetylation as revealed by ChIP using anti-H3K9Ac antibody, coinciding with reduced recruitment of HDACs. Thus, NUR77 and RARβ are genetically and epigenetically regulated by RA and HDAC inhibitors butyrate and SAHA.

NUR77 and RARβ acetylation status was also studied because protein acetylation alters nuclear receptor activity³⁷. Endogenous NUR77 and RARβ in HCT116 cells were immunoprecipitated using anti-NUR77 or anti-RARβ antibody and probed with anti-Ac-lysine antibody in cells treated with RA and/or butyrate or SAHA. The results revealed that SAHA increased acetylated NUR77 and RARβ in HCT116 cells while butyrate only increased acetylated NUR77 (FIG. 32B). However, a combination of RA plus butyrate or SAHA increased acetylation of both receptors (FIG. 32B). Furthermore, miR-22 also increased acetylated NUR77 and RARβ in HCT116 cells (FIG. 32B). To study the effect of RA/butyrate in modulating NUR77 and RARβ transcriptional activity, ChIP-qPCR and RT-PCR were performed to determine receptor binding to transcriptional regulatory regions and mRNA levels of downstream targets, respectively. The data showed that butyrate and SAHA alone or in combination with RA, but not RA alone, reduced NUR77 binding to the BRE and CCND2 promoter regions as well as their corresponding mRNA levels (FIG. 32C). Thus, RA plus HDAC inhibitor treatment reduced NUR77 transcriptional activity. In contrast to NUR77, RARβ binding to the DR5 motif upstream of RARβ and CYP26A1 genes increased by 4 and 2.5 folds following butyrate and SAHA treatment, respectively. Even greater RARβ recruitment was noted in response to combination treatments (FIG. 32D). Therefore, RA and HDAC inhibitor treatment stimulated RARβ transcriptional activity, which may contribute to miR-22 induction as well.

RA/Butyrate and miR-22 Inhibition of Colon Tumor Growth was Accompanied by Silenced HDACs and CCNA2 as Well as Upregulated NUR77 and RARβ in Mice

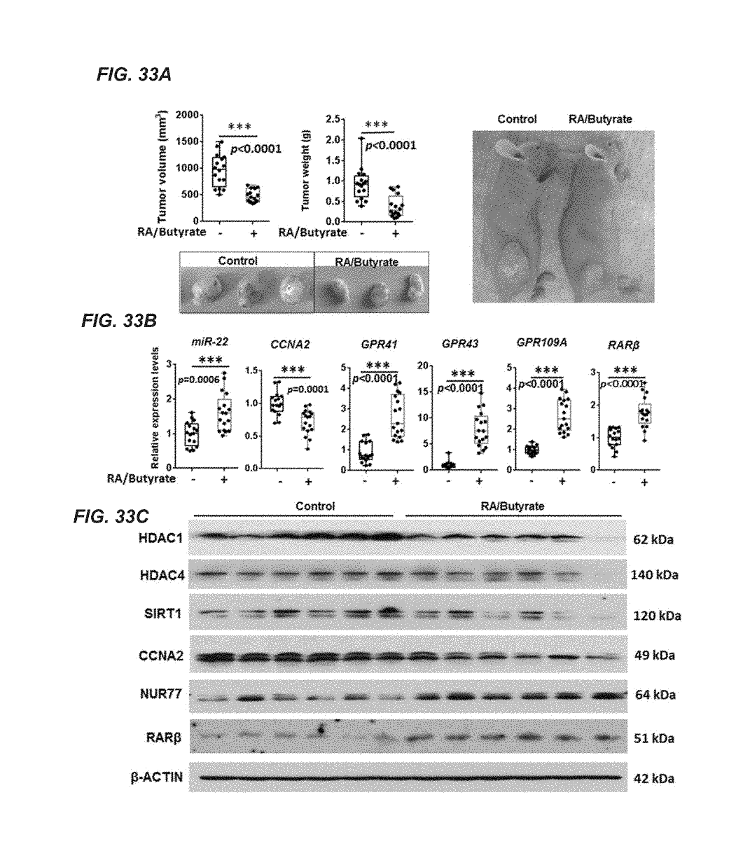

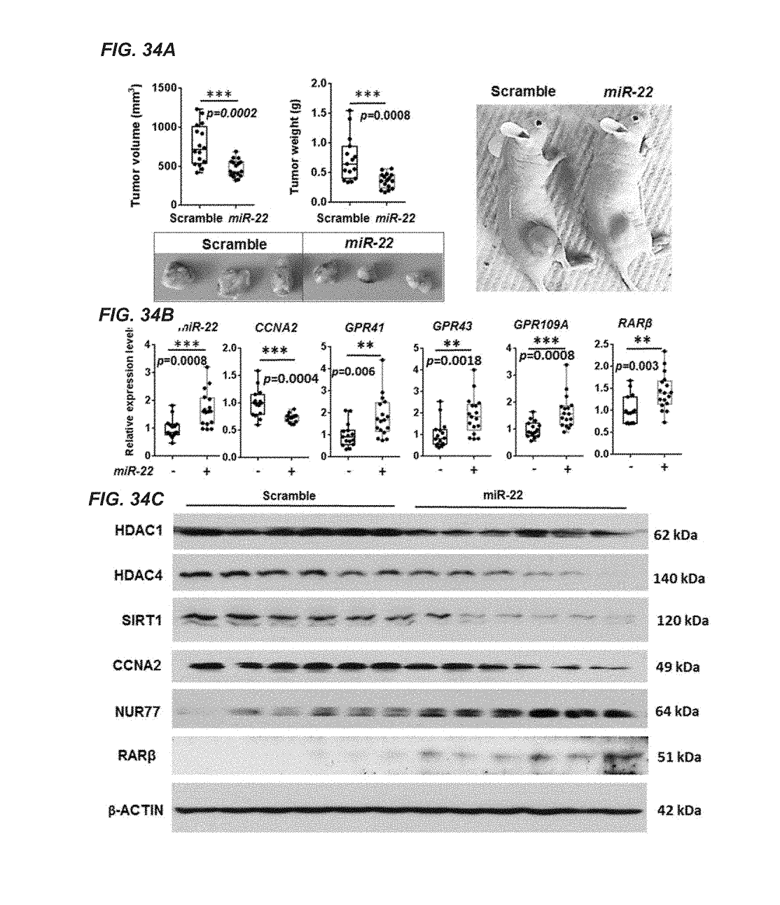

The anti-neoplastic effect of RA/butyrate and miR-22 was studied in mouse xenograft tumor model. RA/butyrate significantly reduced the volume and weight of HCT116-generated tumors. RA/butyrate also upregulated miR-22 and the receptors for RA and SCFAs while reducing CCNA2 (FIGS. 33A and 33B). Moreover, RA/butyrate suppressed HDAC1, HDAC4, SIRT1, and CCNA2 while increasing NUR77 and RARβ (FIG. 33C). Furthermore, adenoviral delivery of miR-22 similarly reduced HCT116-generated tumor volume and weight (FIGS. 34A and B). Consistently, miR-22 silenced HDACs and CCNA2 while upregulating NUR77 and RARβ at the protein levels (FIG. 34C).

### Discussion

For the first time, our data uncovered HDAC1 as a novel miR-22 direct target. Mammalian cells express four classes of HDACs. SAHA, butyrate, trichostatin A, and scriptaid commonly inhibit Class I (HDAC1, 2, 3, and 8) and Class IIa (HDAC4, 5, 7, and 9) ³⁸. Class III includes SIRT1-7 while Class IV consists of HDAC11. It has been shown that miR-22 inhibits liver cancer cell proliferation by directly targeting HDAC4³. In addition, miR-22 directly targets HDAC4 and SIRT1 in rat cardiomyocytes³⁶. Data from our study also demonstrated the ability of miR-22 to suppress HDAC4 and SIRT1 expression in colon cancer cells. Thus, miR-22 has a broad silencing effect on Class I, IIa, and III HDACs.

Our novel data revealed that HDAC inhibitors induce miR-22. Because miR-22 silences multiple HDACs, miR-22 appears a functional HDAC inhibitor. This information provides unique insights into how synthetic and natural HDAC inhibitors, i.e. SAHA and SCFAs, exert their anti-neoplastic effect through miR-22 induction. It is important to mention that all three HDAC inhibitory SCFAs stimulate miR-22 expression in colonocytes. In addition, all three SCFA receptor genes are diminished in colon polyp and cancer specimens. GPR41 is primarily activated by propionate, followed by butyrate and acetate. However, GPR43 can be activated by propionate, butyrate, and acetate at similar rates while GPR109a is butyrate-specific^{15, 39}. Furthermore, bacterial butyrate-generating genes bcoA and buk were also suppressed in polyps and colon cancer specimens. Together, these findings clearly indicate the significance of bacteria-generated SCFAs through miR-22 to protect colon health.

It has been shown that GPR43 serves as an apoptotic inducer and inhibitor of cell cycle progression in colon cancer cell by downregulating PCNA, CCND3, CDK1 and CDK2 while stimulating p21 expression⁴⁰. In addition, GPR109A induces colon cancer cell death by inhibiting BCL2, BCL1-XL, and CCND1 and activating death receptor pathway³⁹. The current study provides another novel mechanism by which SCFAs through microRNA to induce apoptosis.

In addition to HDAC inhibitors, our published data show that bile acids such as chenodeoxycholic acid and vitamin D3 can also induce miR-22, which reduces CCNA2 expression². Consistently, this study revealed that HDAC inhibitors reduced CCNA2. It is important to note that bile acid homeostasis is regulated by both host and bacterial enzymes ^{41, 42, 43}. Thus, our findings support those natural chemicals generated by host and bacteria in the GI tract, specifically RA, SCFAs, and bile acids, can stimulate miR-22 expression. Consistently, FXR deactivation induces spontaneous liver tumorigenesis and increases colon cancer incidence ^{44, 45, 46, 47}. Together, miR-22 appears to be a convergence point for bile acids, vitamin D3, and SCFA signaling pathways to confer their health beneficial effects in the GI tract.

NUR77 can be induced by both mitogens and apoptosis inducers ^{27, 28}. Our published data showed that bile acids also induce NUR77. Depending on the duration of the treatment and concentration of bile acid used, NUR.77 enrichment can be nuclear or cytosolic, suggesting its role in both tissue renewal and apoptosis²⁸. Long-term exposure of intestinal and hepatic cells to secondary bile acids deoxycholic acid (DCA) and lithocholic acid (LCA) can be detrimental. Hydrophobic DCA and LCA, which are elevated in obese patients, damage DNA, stimulate inflammatory signaling, and increase oncogenic nuclear NUR77²⁸. Furthermore, because colon cancer patients also have reduced SCFAs and SCFA receptors, elevated nuclear NUR77 may not be able to be exported to the cytosol. Therefore, dysbiosis-associated bile acid dysregulation synthesis and SCFA deficiency likely play a crucial role in colon carcinogenesis through nuclear NUR77 elevation.

It has been shown that nuclear export of NUR77 is regulated by phosphorylation. Several studies have shown that JNK1/2 and CHK2-mediated phosphorylation leads to NUR77 nuclear export whereas ERK1/2-mediated phosphorylation promotes NUR77 nuclear retention ^{24, 48, 49}. Consistently, our data showed that RA/butyrate and RA/SAHA induced cleaved caspase 3 and NUR77, which was accompanied by JNK1/2 activation. Another study showed that NUR77 expression and protein stability are modulated via acetylation by p300 and HDAC1, but NUR77 acetylation modification does not alter its subcellular localization ⁵⁰. However, our data revealed that the induction of cytosolic NUR77 was miR-22 dependent. Additionally, NUR77 and RARβ displayed greater acetylation in response to HDAC inhibitor treatment. The mechanism by which miR-22-induced cytosolic NUR77 remains to be further investigated in colon cancer cells.

RA plus butyrate or SAHA-induced NUR77 acetylation and cytosolic enrichment was accompanied by reduced recruitment of NUR77 to the regulatory region of its target genes, i.e. BRE and CCND2, which supports a shift from its proliferative to apoptotic function. In contrast, acetylated RARβ induced by RA plus butyrate or SAHA have increased transcriptional activity, consistent the finding that RARβ is involved in inducing miR-22 expression. Moreover, RARβ is a tumor suppressor, and it is self-regulated¹⁷. Together, increased transcriptional activity of RARβ would enhance its nuclear tumor suppressive function. Additionally, increased production of RARβ can also interact with NUR77 to execute its apoptotic effect²².

In conclusion, we uncovered the underlying interaction between SAHA as well as SCFAs and RA in inducing miR-22 to silence multiple HDACs, which are involved in transcriptional and post-translational modification of nuclear receptors NUR77 and RARβ.

Excitingly, mouse xenograft model revealed remarkable effectiveness of miR-22 and combined RA plus butyrate in inhibiting colonic HCT116 tumor growth. The data generated using cell lines, mice, as well as human specimens consistently point to miR-22 as a novel regulatory mechanism linking diet and colon cancer prevention or treatment. Our findings will guide future use of probiotics and dietary interventions to not only maintain GI health but also colon cancer treatment. Lastly, miR-22-mediated NUR77 and RARβ induction appears a promising novel therapeutic approach for colon cancer.

### Materials and Methods

### Cell Culture

HCT116 cells (American Type Culture Collection) were maintained in McCoy's 5a medium from Gibco (Life Technologies, Carlsbad, Calif.) with 10% FBS. Cells were plated (1×10⁶ cells per 60-mm dish, 2×10⁵ cells per 6-well plates, and 1×10⁵ cells per 24-well plates) overnight prior to treatment or transfection.

mRNA Quantification

Total RNA was extracted with TRIzol Reagent (Invitrogen, Carlsbad, Calif.), and cDNA was generated using High Capacity RNA-to-cDNA Kit (Applied Biosystems, Carlsbad, Calif.). qRT-PCR was performed on ABI 7900HT Fast Real time PCR system using Power SYBR® Green PCR Master Mix (Applied Biosystems, Carlsbad, Calif.).

### MTT Assay

HCT116 cells were seeded in 24-well plates and treated with sodium butyrate (5 mM, Sigma), sodium propionate (10 mM, Sigma-Aldrich, St. Louis, Mo.), valerate (10 mM, Sigma), SAHA (5 μM, Sigma-Aldrich, St. Louis, Mo.), and/or RA (10 μM, Sigma-Aldrich, St. Louis, Mo.). Cells were stained with 3-(4,5-dimethylthiazol-2-O-2,5-diphenyltetrazolium bromide (MTT, Sigma-Aldrich, St. Louis, Mo.) and measured at 570 nm with Microplate Reader (Biotek, Winooski, Vt.).

### Plasmid Construction and Luciferase Reporter Assay

Nuclear receptor binding motifs including DR5 motif (GGGTCAGGGCCAGTTCA (SEQ ID NO:3), −1585 to −1568 bp), ER6 (TGGACAGAGAGAAGGTCA (SEQ ID NO:4), −1339 to −1321 bp), DR1 (TGGCCTGTCACCC (SEQ ID NO:5), −1101 to −1088 bp), IR1 motif (GGGTCAGTGCCCT (SEQ ID NO:6), −1025 to −1012 bp), and DR3 motif (TGAACCCTGTGGCCT (SEQ ID NO:7), −954 to −939 bp), which are found in the miR-22 regulatory region, were cloned into the PGL3 vector as described previously². Motif (GCTGTCATGGTGCCAGAGAGTTGATGGAGCAGCTGGT (SEQ ID NO:8)) located 4 bp away from the IR1 motif was also cloned as a negative control (PGL3-Neg). The 3′ untranslated region (3′-UTR) of the HDAC1 gene containing the putative binding sites for miR-22 was cloned into the psiCHECK2 vector (Promega, Madison, Wis.) using Noti and XhoI. For luciferase reporter assays, cells were transfected with PGL3-DR5, PGL3-ER6, PGL3-DR1, PGL3-IR1, PGL3-DR3, or PGL3-Neg using Lipofectamine 2000 (Life Technologies, Carlsbad, Calif.) for 6 hours. Then, fresh medium containing RA (10 μM) and butyrate (5 mM) or DMSO was replaced for 24-hours treatment. For the 3′-UTR luciferase assay, cells were co-transfected with miR-22 mimics (20 nM, Life Technologies, Carlsbad, Calif.) or miR-22 inhibitors (50 nM, Life Technologies, Carlsbad, Calif.) and psiCHECK2-HDAC1 using Lipofectamine 2000 (Life Technologies, Carlsbad, Calif.). Twenty-four hours after transfection, cells were harvested for Firefly and Renilla luciferase assay using the Dual-luciferase Reporter system (Promega, Madison, Wis.). Renilla luciferase activity was standardized to firefly luciferase activity.

### Western Blotting and Immunoprecipitation

Western blot and immunoprecipitation were performed as described previously²². Specific antibodies used included anti-NUR77, RARβ, SIRT1, β-actin, and IgG (Santa Cruz Biotechnology, Santa Cruz, Calif.), phospho-JNK1/2, total JNK1/2, cleaved caspase3, HDAC1 and HDAC4 (Cell Signaling Technology, Beverly, Mass.), and CCNA2 (Novus Biologicals, Littleton, Colo.). To detect protein acetylation of NUR77 and RARβ, cell lysates (500 μg) were incubated with anti-NUR77, RARβ, or IgG antibody (1 μg), precipitated by adding Dynabeads (30 μl, Life Technologies, Carlsbad, Calif.), and probed with anti-Acetyl-lysine antibody (Cell Signaling Technology, Beverly, Mass.). To study the interaction between RARβ and NUR77, whole-cell lysates were precipitated with anti-RARβ or NUR77 antibody followed by Western blot using anti-NUR77 or RARβ antibody.

Chromatin immunoprecipitation (ChIP)-qPCR ChIP-qPCR was performed as described previously²¹. Briefly, chromatin lysate was precleared before incubation with an anti-H3K9AC antibody (Millipore. Billerica, Mass.), anti-HDAC1 and anti-HDAC4 (Cell signaling technology, Beverly, Mass.) or anti-SIRT1 antibody (Santa Cruz Biotechnology, Santa Cruz, Calif.). IgG (Santa Cruz Biotechnology, Santa Cruz, Calif.) and RNA Polymerase II antibody (Millipore, Billerica, Mass.) were used as negative and positive controls, respectively. Samples were incubated with Dynase beads at 4° C. overnight followed by de-crosslinking and purification. DNA fragments generated served as templates for qPCR using Power SYBR1 Green PCR Master Mix.

### Immunostaining and Confocal Microscopy

HCT116 cells were grown on poly-L-lysine-coated, glass coverslips and treated with 10 μM RA, 5 mM butyrate, 5 μM SAHA and combination of RA plus butyrate or SAHA for 48 hours. Following treatment, cells were immunostained with anti-NUR77 (Abcam, Cambridge, Mass.) and anti-RARβ (Santa Cruz Biotechnology, Santa Cruz, Calif.) antibodies followed by Alexa Fluor® 488 anti-rabbit IgG and Alexa Fluor® 594 anti-goat IgG (Invitrogen, Carlsbad, Calif.). To study the effect of miR-22 in induction of cytosolic NUR77 by RA/butyrate, adenoviral-delivery of miR-22 inhibitors tagged with green fluorescence (adeno-miR-22 inhibitor-GFP, Applied Biological Materials Inc, Richond, BC) were used to infect HCT116 cells. At 48 hours post adenoviral infection, cells were treated with RA (10 μM) plus butyrate (5 mM) or DMSO for 24 hours. Treated cells were immunostained with anti-NUR77 antibody (Abcam, Cambridge, Mass.) followed by Alexa Fluor® 594 anti-rabbit IgG (Invitrogen, Carlsbad, Calif.). Cells were mounted in ProLong® Gold Antifade Reagent with DAPI (4′,6-diamidino-2-phenylindole, Life Technologies, Carlsbad, Calif.) and imaged under Keyence BZ-9000 microscope.

### Human Specimens

Frozen cancer and adjacent benign specimens, as confirmed by histological evaluation, were obtained from the Translational Pathology Core Laboratory Shared Resource at UCLA. In addition, fresh colon polyps and normal adjacent tissues were obtained from the Gastrointestinal (GI) Biorepository at UC Davis.

### Quantification of Bacterial DNA

Genomic DNA was extracted from colon polyps and adenocarcinomas and their adjacent normal tissues using ZR Fecal DNA MiniPrep Kit (Zymo Research, Irvine, Calif.). DNA (50 ng) was amplified using primers specific for butyryl-coenzyme-A-CoA transferase (bcoA) and butyrate kinase (buk) genes^{33,34}. A dissociation step was included to analyze the melting profile of amplified products. In parallel, qPCR was done using ten-fold serially diluted synthetic DNA fragments of bacterial genes with known concentrations. Bacterial DNA concentration was calculated using standard curves of diluted synthetic DNA fragments.

### Mouse Xenograft Tumor Model

Athymic nude mice (female, 6 weeks old, Jackson Laboratory) were subcutaneously injected by HCT116 cells (1×10⁶) in the flank region. When tumor size reached 100 mm³ of cross-sectional area, a combination of RA plus butyrate or adenoviral-expressing miR-22 (Ad-miR-22) was administered. For RA plus butyrate treatment, mice received RA (2.5 μg/g body weight) and butyrate (1.2 mg/g body weight) intraperitoneally (IP) 5 times a week for 2 weeks. For Ad-miR-22 treatment, mice received Ad-miR-22 (1×10⁹ Pfu) intratumorally (IT) twice a week for 2 weeks. Control groups received 1% DMSO in PBS and adenovirus scramble, respectively. Tumor dimensions were measured and tumor volume was calculated using V (volume)=W (width)²×L (length)/2. All experimental protocols were approved by the University of California Davis Animal Care and Use Committee.

### Statistical Analysis

Data are presented as mean±SD. The difference between two groups was analyzed with Student's t-test. p<0.05 was considered statistically significant.

### REFERENCES

1. Tsuchiya N, Izumiya M, Ogata-Kawata H, Okamoto K, Fujiwara Y, Nakai M, et al. Tumor Suppressor miR-22 Determines p53-Dependent Cellular Fate through Post-transcriptional Regulation of p21. Cancer Res 2011, 71(13): 4628-4639.
2. Yang F, Hu Y, Liu H X, Wan Y J. MiR-22-silenced cyclin A expression in colon and liver cancer cells is regulated by bile acid receptor. J Biol Chem 2015, 290(10): 6507-6515.
3. Zhang J, Yang Y, Yang T, Liu Y, Li A, Fu S, et al. microRNA-22, downregulated in hepatocellular carcinoma and correlated with prognosis, suppresses cell proliferation and tumourigenicity. Br J Cancer 2010, 103(8): 1215-1220.
4. Xiong J, Du Q, Liang Z. Tumor-suppressive microRNA-22 inhibits the transcription of E-box-containing c-Myc target genes by silencing c-Myc binding protein. Oncogene 2010, 29(35): 4980-4988.
5. Yamakuchi M, Yagi S, Ito T, Lowenstein C J. MicroRNA-22 regulates hypoxia signaling in colon cancer cells. PloS one 2011, 6(5): e20291.
6. Xiong R I, Yu D K, Wei N, Fu H J, Cai T J, Huang Y Y, et al. An estrogen receptor alpha suppressor, microRNA-22, is downregulated in estrogen receptor alpha-positive human breast cancer cell lines and clinical samples. FEBS J 2010, 277(7): 1684-1694.
7. Ling B, Wang G X, Long G, Qiu J H, Hu Z L. Tumor suppressor miR-22 suppresses lung cancer cell progression through post-transcriptional regulation of ErbB3. J Cancer Res Clin 2012, 138(8): 1355-1361.
8. Lionetti M, Agnelli L, Mosca L, Fabris S, Andronache A, Todoerti K, et al. Integrative high-resolution microarray analysis of human myeloma cell lines reveals deregulated miRNA expression associated with allelic imbalances and gene expression profiles. Genes chromosomes & cancer 2009, 48(6): 521-531.
9. Petrosino J M, Disilvestro D, Ziouzenkova O. Aldehyde dehydrogenase 1A1: friend or foe to female metabolism? Nutrients 2014, 6(3): 950-973.
10. Duester G. Retinoic acid synthesis and signaling during early organogenesis. Cell 2008, 134(6): 921-931.
11. Waldecker M, Kautenburger T, Daumann H, Busch C, Schrenk D. Inhibition of histone-deacetylase activity by short-chain fatty acids and some polyphenol metabolites formed in the colon. J Nutr Biochem 2008, 19(9): 587-593.
12. Marks P, Rifkind R A, Richon V M, Breslow R, Miller T, Kelly W K. Histone deacetylases and cancer: causes and therapies. Nat Rev Cancer 2001, 1(3): 194-202.
13. Donohoe D R, Holley D, Collins L B, Montgomery S A, Whitmore A C, Hillhouse A, et al. A gnotobiotic mouse model demonstrates that dietary fiber protects against colorectal tumorigenesis in a microbiota- and butyrate-dependent manner. Cancer Discov 2014, 4(12): 1387-1397.
14. O'Keefe S J. Diet, microorganisms and their metabolites, and colon cancer. Nat Rev Gastroenterol Hepatol 2016, 13(12): 691-706.
15. den Besten G, van Eunen K, Groen A K, Venema K, Reijngoud D J, Bakker B M. The role of short-chain fatty acids in the interplay between diet, gut microbiota, and host energy metabolism. J Lipid Res 2013, 54(9): 2325-2340.
16. Singh N, Gurav A, Sivaprakasam S, Brady E, Padia R, Shi H, et al. Activation of Gpr109a, receptor for niacin and the commensal metabolite butyrate, suppresses colonic inflammation and carcinogenesis. Immunity 2014, 40(1): 128-139.
17. Bushue N, Wan Y J. Retinoid pathway and cancer therapeutics. Adv Drug Deliv Rev 2010, 62(13): 1285-1298.
18. di Masi A, Leboffe L, De Marinis E, Pagano F, Cicconi L, Rochette-Egly C, et al. Retinoic acid receptors: from molecular mechanisms to cancer therapy. Mol Aspects Med 2015, 41: 1-115.
19. Liu H X, Ly I, Hu Y, Wan Y J. Retinoic acid regulates cell cycle genes and accelerates normal mouse liver regeneration. Biochem Pharmacol 2014, 91(2): 256-265.
20. Liu H X, Hu Y, Wan Y J. Microbiota and bile acid profiles in retinoic acid-primed mice that exhibit accelerated liver regeneration. Oncotarget 2016, 7(2): 1096-1106.
21. Hu Y, Liu H X, He Y, Fang Y, Fang J, Wan Y J. Transcriptome profiling and genome-wide DNA binding define the differential role of fenretinide and all-trans RA in regulating the death and survival of human hepatocellular carcinoma Huh? cells. Biochem Pharmacol 2013, 85(7): 1007-1017.
22. Yang H, Zhan Q, Wan Y J. Enrichment of Nur77 mediated by retinoic acid receptor beta leads to apoptosis of human hepatocellular carcinoma cells induced by fenretinide and histone deacetylase inhibitors. Hepatology 2011, 53(3): 865-874.
23. Yang H, Bushue N, Bu P, Wan Y J. Induction and intracellular localization of Nur77 dictate fenretinide-induced apoptosis of human liver cancer cells. Biochem Pharmacol 2010, 79(7): 948-954.
24. Yang H, Nie Y, Li Y, Wan Y J. ERK1/2 deactivation enhances cytoplasmic Nur77 expression level and improves the apoptotic effect of fenretinide in human liver cancer cells. Biochem Pharmacol 2011, 81(7): 910-916.
25. Touma S E, Goldberg J S, Moench P, Guo X, Tickoo S K, Gudas L J, et al. Retinoic acid and the histone deacetylase inhibitor trichostatin a inhibit the proliferation of human renal cell carcinoma in a xenograft tumor model. Clin Cancer Res 2005, 11(9): 3558-3566.
26. Minucci S, Horn V, Bhattacharyya N, Russanova V, Ogryzko V V, Gabriele L, et al. A histone deacetylase inhibitor potentiates retinoid receptor action in embryonal carcinoma cells. Proc Natl Acad Sci USA 1997, 94(21): 11295-11300.
27. Moll U M, Marchenko N, Zhang X K. p53 and Nur77/TR3—transcription factors that directly target mitochondria for cell death induction. Oncogene 2006, 25(34): 4725-4743.
28. Hu Y, Chau T, Liu H X, Liao D, Keane R, Nie Y, et al. Bile acids regulate nuclear receptor (Nur77) expression and intracellular location to control proliferation and apoptosis. Mol Cancer Res 2015, 13(2): 281-292.
29. Kolluri S K, Bruey-Sedano N, Cao X, Lin B, Lin F, Han Y H, et al. Mitogenic effect of orphan receptor TR3 and its regulation by MEKK1 in lung cancer cells. Mol Cell Biol 2003, 23(23): 8651-8667.
30. Lee S O, Jin U H, Kang J H, Kim S B, Guthrie A S, Sreevalsan S, et al. The orphan nuclear receptor NR4A1 (Nur77) regulates oxidative and endoplasmic reticulum stress in pancreatic cancer cells. Mol Cancer Res 2014, 12(4): 527-538.
31. Lin B, Kolluri S K, Lin F, Liu W, Han Y H, Cao X, et al. Conversion of Bcl-2 from protector to killer by interaction with nuclear orphan receptor Nur77/TR3. Cell 2004, 116(4): 527-540.
32. Abend A, Kehat I. Histone deacetylases as therapeutic targets—From cancer to cardiac disease. Pharmacol Therapeut 2015, 147: 55-62.
33. O'Keefe S J, Li J V, Lahti L, Ou J, Carbonero F, Mohammed K, et al. Fat, fibre and cancer risk in African Americans and rural Africans. Nat Commun 2015, 6: 6342.
34. Louis P, Duncan S H, McCrae S I, Millar J, Jackson M S, Flint H J. Restricted distribution of the butyrate kinase pathway among butyrate-producing bacteria from the human colon. J Bacteriol 2004, 186(7): 2099-2106.
35. Kiefer J, Beyer-Sehlmeyer G, Pool-Zobel B L. Mixtures of SCFA, composed according to physiologically available concentrations in the gut lumen, modulate histone acetylation in human HT29 colon cancer cells. Br J Nutr 2006, 96(5): 803-810.
36. Huang Z P, Chen J, Seok H Y, Zhang Z, Kataoka M, Hu X, et al. MicroRNA-22 regulates cardiac hypertrophy and remodeling in response to stress. Circ Res 2013, 112(9): 1234-1243.
37. Baek S H, Rosenfeld M G. Nuclear receptor coregulators: their modification codes and regulatory mechanism by translocation. Biochem Bioph Res Co 2004, 319(3): 707-714.
38. Dokmanovic M, Clarke C, Marks P A. Histone deacetylase inhibitors: overview and perspectives. Mol Cancer Res 2007, 5(10): 981-989.
39. Thangaraju M, Cresci G A, Liu K, Ananth S, Gnanaprakasam J P, Browning D D, et al. GPR109A Is a G-protein-Coupled Receptor for the Bacterial Fermentation Product Butyrate and Functions as a Tumor Suppressor in Colon. Cancer Res 2009, 69(7): 2826-2832.
40. Tang Y, Chen Y, Jiang H, Robbins G T, Nie D. G-protein-coupled receptor for short-chain fatty acids suppresses colon cancer. Int J Cancer 2011, 128(4): 847-856.
41. Tsuei J, Chau T, Mills D, Wan Y J. Bile acid dysregulation, gut dysbiosis, and gastrointestinal cancer. Exp Biol Med (Maywood) 2014, 239(11): 1489-1504.
42. Li T, Chiang J Y. Bile acids as metabolic regulators. Curr Opin Gastroenterol 2015, 31(2): 159-165.
43. Joyce S A, MacSharry J, Casey P G, Kinsella M, Murphy E F, Shanahan F, et al. Regulation of host weight gain and lipid metabolism by bacterial bile acid modification in the gut. Proc Natl Acad Sci USA 2014, 111(20): 7421-7426.
44. Modica S, Murzilli S, Salvatore L, Schmidt D R, Moschetta A. Nuclear bile acid receptor FXR protects against intestinal tumorigenesis. Cancer Res 2008, 68(23): 9589-9594.
45. Maran R R, Thomas A, Roth M, Sheng Z, Esterly N, Pinson D, et al. Farnesoid X receptor deficiency in mice leads to increased intestinal epithelial cell proliferation and tumor development. J Pharmacol Exp Ther 2009, 328(2): 469-477.
46. Yang F, Huang X, Yi T, Yen Y, Moore D D, Huang W. Spontaneous development of liver tumors in the absence of the bile acid receptor farnesoid X receptor. Cancer Res 2007, 67(3): 863-867.
47. Kim I, Morimura K, Shah Y, Yang Q, Ward J M, Gonzalez F J. Spontaneous hepatocarcinogenesis in farnesoid X receptor-null mice. Carcinogenesis 2007, 28(5): 940-946.
48. Yao L M, He J P, Chen H Z, Wang Y, Wang W J, Wu R, et al. Orphan receptor TR3 participates in cisplatin-induced apoptosis via Chk2 phosphorylation to repress intestinal tumorigenesis. Carcinogenesis 2012, 33(2): 301-311.
49. Han Y H, Cao X, Lin B, Lin F, Kolluri S K, Stebbins J, et al. Regulation of Nur77 nuclear export by c-Jun N-terminal kinase and Akt. Oncogene 2006, 25(21): 2974-2986.
50. Kang S A, Na H, Kang H J, Kim S H, Lee M H, Lee M O. Regulation of Nur77 protein turnover through acetylation and deacetylation induced by p300 and HDAC1. Biochem Pharmacol 2010, 80(6): 867-873.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### VII. Exemplary Embodiments

Exemplary embodiments provided in accordance with the presently disclosed subject matter include, but are not limited to, the claims and the following embodiments:

1. A method for treating cancer in a subject, the method comprising administering to the subject a therapeutically effective amount of a retinoid and a histone deacetylase (HDAC) inhibitor.
2. The method of embodiment 1, wherein the HDAC inhibitor is selected from the group consisting of a short-chain fatty acid (SCFA), suberanilohydroxamic acid (SAHA), and a combination thereof.
3. The method of embodiment 1 or 2, wherein the retinoid is selected from the group consisting of retinoic acid (RA), retinol, retinal, isotretinoin, alltretinoin, etretinate, acitretin, tazarotene, bexarotene, adapalene, seletinoid G, a retinyl ester, fenretinide, derivatives thereof, and a combination thereof.
4. The method of embodiment 3, wherein the retinyl ester is selected from the group consisting of retinyl acetate, retinyl butyrate, retinyl propionate, retinyl palmitate, and a combination thereof.
5. The method of any one of embodiments 1 to 4, wherein the retinoid is RA.
6. The method of any one of embodiments 2 to 5, wherein the SCFA is selected from the group consisting of formate, acetate, propionate, butyrate, isobutyrate, valerate, isovalerate, and a combination thereof.
7. The method of any one of embodiments 2 to 5, wherein the HDAC inhibitor is SAHA.
8. The method of any one of embodiments 2 to 6, wherein the SCFA is butyrate.
9. The method of any one of embodiments 2 to 6, wherein the SCFA is propionate.
10. The method of any one of embodiments 2 to 6, wherein the SCFA is valerate.
11. The method of any one of embodiments 1 to 10, wherein the cancer is liver cancer.
12. The method of any one of embodiments 1 to 10, wherein the cancer is colon cancer.
13. The method of embodiment 12, wherein the subject has one or more colon polyps.
14. The method of any one of embodiments 1 to 13, wherein the retinoid and HDAC inhibitor are administered orally.
15. The method of any one of embodiments 1 to 14, wherein a sample is obtained from the subject.
16. The method of embodiment 15, wherein the sample comprises blood, tissue, or a combination thereof.
17. The method of embodiment 16, wherein the tissue comprises cancer tissue.
18. The method of embodiment 16, wherein the tissue comprises normal tissue.
19. The method of any one of embodiments 15 to 18, wherein the level of one or more biomarkers is measured in the sample.
20. The method of embodiment 19, wherein at least one of the one or more biomarkers is a microRNA (miR).
21. The method of embodiment 20, wherein the miR is selected from the group consisting of miR-22, miR-34a, and a combination thereof.
22. The method of any one of embodiments 19 to 21, wherein the measured level of the one or more biomarkers in the sample is abnormal compared to a reference sample.
23. The method of embodiment 22, wherein the reference sample is obtained from the subject.
24. The method of embodiment 22, wherein the reference sample is obtained from a different subject or a population of subjects.
25. The method of any one of embodiments 19 to 24, wherein the level of the one or more biomarkers is measured before the retinoid and HDAC inhibitor are administered to the subject.
26. The method of any one of embodiments 1 to 25, wherein the administration of the retinoid and HDAC inhibitor to the subject improves one or more symptoms of cancer in the subject.
27. A method for treating cancer in a subject, the method comprising administering to the subject a therapeutically effective amount of a microRNA (miR) or a mimic thereof, wherein the miR is selected from the group consisting of miR-22, miR-34a, and a combination thereof.
28. The method of embodiment 27, wherein the miR-22 comprises a nucleotide sequence having at least 90% sequence identity to SEQ ID NO:1.
29. The method of embodiment 27, wherein the miR-34a comprises a nucleotide sequence having at least 90% sequence identity to SEQ ID NO:2.
30. The method of any one of embodiment 27 to 29, wherein the miR or mimic thereof is virally expressed.
31. The method of any one of embodiments 27 to 30, wherein the cancer is liver cancer.
32. The method of any one of embodiments 27 to 30, wherein the cancer is colon cancer.
33. The method of embodiment 32, wherein the subject has one or more colon polyps.
34. The method of any one of embodiments 27 to 33, wherein a sample is obtained from the subject.
35. The method of embodiment 34, wherein the sample comprises blood, tissue, or a combination thereof.
36. The method of embodiment 35, wherein the tissue comprises cancer tissue.
37. The method of embodiment 35, wherein the tissue comprises normal tissue.
38. The method of any one of embodiments 34 to 37, wherein the level of one or more biomarkers is measured in the sample.
39. The method of embodiment 38, wherein at least one of the one or more biomarkers is a microRNA (miR).
40. The method of embodiment 39, wherein the miR is selected from the group consisting of miR-22, miR-34a, and a combination thereof.
41. The method of any one of embodiments 38 to 40, wherein the measured level of the one or more biomarkers in the sample is abnormal compared to a reference sample.
42. The method of embodiment 41, wherein the reference sample is obtained from the subject.
43. The method of embodiment 41, wherein the reference sample is obtained from a different subject or a population of subjects.
44. The method of any one of embodiments 38 to 43, wherein the level of the one or more biomarkers is measured before the miR or mimic thereof is administered to the subject.
45. The method of any one of embodiments 27 to 44, wherein the administration of the miR or the mimic thereof to the subject improves one or more symptoms of cancer in the subject.
46. A method for treating a metabolic disease in a subject, the method comprising administering to the subject a therapeutically effective amount of a retinoid and a histone deacetylase (HDAC) inhibitor.
47. The method of embodiment 46, wherein the HDAC inhibitor is selected from the group consisting of a short-chain fatty acid (SCFA), suberanilohydroxamic acid (SAHA), and a combination thereof.
48. The method of embodiment 46 or 47, wherein the retinoid is selected from the group consisting of retinoic acid (RA), retinol, retinal, isotretinoin, alltretinoin, etretinate, acitretin, tazarotene, bexarotene, adapalene, seletinoid G, a retinyl ester, fenretinide, derivatives thereof, and a combination thereof.
49. The method of embodiment 48, wherein the retinyl ester is selected from the group consisting of retinyl acetate, retinyl butyrate, retinyl propionate, retinyl palmitate, and a combination thereof.
50. The method of any one of embodiments 46 to 49, wherein the retinoid is RA.
51. The method of any one of embodiments 47 to 50, wherein the SCFA is selected from the group consisting of formate, acetate, propionate, butyrate, isobutyrate, valerate, isovalerate, and a combination thereof.
52. The method of any one of embodiments 47 to 50, wherein the HDAC inhibitor is SAHA.
53. The method of any one of embodiments 47 to 51, wherein the SCFA is butyrate.
54. The method of any one of embodiments 47 to 51, wherein the SCFA is propionate.
55. The method of any one of embodiments 47 to 51, wherein the SCFA is valerate.
56. The method of any one of embodiments 46 to 55, wherein the metabolic disease is selected from the group consisting of non-alcoholic steatohepatitis (NASH), non-alcoholic fatty liver disease (NAFLD), diabetes, obesity, and a combination thereof.
57. The method of any one of embodiments 46 to 56, wherein the retinoid and HDAC inhibitor are administered orally.
58. The method of any one of embodiments 46 to 57, wherein a sample is obtained from the subject.
59. The method of embodiment 58, wherein the sample comprises blood, tissue, or a combination thereof.
60. The method of embodiment 59, wherein the tissue comprises diseased tissue.
61. The method of embodiment 59, wherein the tissue comprises normal tissue.
62. The method of any one of embodiments 58 to 61, wherein the level of one or more biomarkers is measured in the sample.
63. The method of embodiment 62, wherein the one or more biomarkers is selected from the group consisting of a miR, FGF21, FGFR1c, Beta-klotho, blood glucose, aspartate aminotransferase (AST), alanine aminotransferase (ALT), the ratio of AST to ALT, gamma-glutamyl transferase (GGT), the aspartate to platelet ratio index (APRI), alkaline phosphatase (AP), bilirubin, ferritin, alpha-smooth muscle actin (αSMA), procollagen α1 (procol1), transforming growth factor-β (TGFβ), monocyte chemoattractant protein-1 (MCP1), interleukin-1β(IL-1b), tumor necrosis factor alpha (TNFα), connective tissue growth factor (CTGF), platelet derived growth factor receptor beta (PDGFRβ), and a combination thereof.
64. The method of embodiment 63, wherein the miR is selected from the group consisting of miR-22, miR-34a, and a combination thereof.
65. The method of any one of embodiments 62 to 64, wherein the measured level of the one or more biomarkers in the sample is abnormal compared to a reference sample.
66. The method of embodiment 65, wherein the reference sample is obtained from the subject.
67. The method of embodiment 65, wherein the reference sample is obtained from a different subject or a population of subjects.
68. The method of any one of embodiments 62 to 67, wherein the level of the one or more biomarkers is measured before the retinoid and HDAC inhibitor are administered to the subject.
69. The method of any one of embodiments 46 to 68, wherein the administration of the retinoid and HDAC inhibitor to the subject improves one or more symptoms of the metabolic disease in the subject.
70. The method of any one of embodiments 1 to 69, further comprising administering a starch to the subject.
71. The method of any one of embodiments 1 to 70, further comprising administering a probiotic agent and/or a prebiotic agent to the subject.
72. The method of embodiment 71, wherein the probiotic comprises a bacterium that produces an SCFA.
73. The method of embodiment 71, wherein the prebiotic comprises apple pectin, an inulin, or a combination thereof 74. The method of any one of embodiments 1 to 73, further comprising administering a delivery-enhancing agent to the subject.
75. The method of embodiment 74, wherein the delivery-enhancing agent is selected from the group consisting of a cyclodextrin, a hepatitis E virus-like particle, an inactivated yeast, an inactivated bacterium, polyvinyl acetate (PVA), an inulin or an ester thereof, and a combination thereof 76. The method of embodiment 75, wherein the HDAC inhibitor and the retinoid are packaged into PVA at a HDAC inhibitor-retinoid ratio of about 1:50 to about 1:1,000 by weight.
77. The method of embodiment 75, wherein the inulin ester is selected from the group consisting of an inulin butyrate ester, an inulin propionate ester, and a combination thereof 78. A pharmaceutical composition comprising a retinoid, a histone deacetylase (HDAC) inhibitor, and a pharmaceutically acceptable carrier.
79. The pharmaceutical composition of embodiment 78, wherein the HDAC inhibitor is selected from the group consisting of a short-chain fatty acid (SCFA), suberanilohydroxamic acid (SAHA), and a combination thereof.
80. The pharmaceutical composition of embodiment 78 or 79, wherein the retinoid is selected from the group consisting of retinoic acid (RA), retinol, retinal, isotretinoin, alltretinoin, etretinate, acitretin, tazarotene, bexarotene, adapalene, seletinoid G, a retinyl ester, fenretinide, derivatives thereof, and a combination thereof.
81. The pharmaceutical composition of embodiment 80, wherein the retinyl ester is selected from the group consisting of retinyl acetate, retinyl butyrate, retinyl propionate, retinyl palmitate, and a combination thereof.
82. The pharmaceutical composition of any one of embodiments 78 to 80, wherein the retinoid is RA.
83. The pharmaceutical composition of embodiment 80 or 82, wherein the concentration of RA is about 10 μM.
84. The pharmaceutical composition of any one of embodiments 79 to 83, wherein the SCFA is selected from the group consisting of formate, acetate, propionate, butyrate, isobutyrate, valerate, isovalerate, and a combination thereof 85. The pharmaceutical composition of any one of embodiments 79 to 83, wherein the HDAC inhibitor is SAHA.
86. The pharmaceutical composition of embodiment 85, wherein the concentration of SAHA is about 5 μM.
87. The pharmaceutical composition of any one of embodiments 79 to 84, wherein the SCFA is butyrate.
88. The pharmaceutical composition of embodiment 87, wherein the concentration of butyrate is about 5 mM.
89. The pharmaceutical composition of any one of embodiments 79 to 84, wherein the SCFA is propionate.
90. The pharmaceutical composition of embodiment 89, wherein the concentration of propionate is about 10 mM.
91. The pharmaceutical composition of any one of embodiments 79 to 84, wherein the SCFA is valerate.
92. The pharmaceutical composition of embodiment 91, wherein the concentration of valerate is about 10 mM.
93. The pharmaceutical composition of any one of embodiments 78 to 92, further comprising a starch.
94. The pharmaceutical composition of any one of embodiments 78 to 93, further comprising a probiotic agent and/or a prebiotic agent.
95. The pharmaceutical composition of embodiment 94, wherein the probiotic comprises a bacterium that produces an SCFA.
96. The pharmaceutical composition of embodiment 94, wherein the prebiotic comprises apple pectin, an inulin, or a combination thereof 97. The pharmaceutical composition of any one of embodiments 78 to 96, further comprising a delivery-enhancing agent.
98. The pharmaceutical composition of embodiment 97, wherein the delivery-enhancing agent is selected from the group consisting of a cyclodextrin, a hepatitis E virus-like particle, an inactivated yeast, an inactivated bacterium, polyvinyl acetate (PVA), an inulin or an ester thereof, and a combination thereof 99. The pharmaceutical composition of embodiment 98, wherein the HDAC inhibitor and the retinoid are packaged into PVA at a HDAC inhibitor-retinoid ratio of about 1:50 to about 1:1,000 by weight.
100. The pharmaceutical composition of embodiment 98, wherein the inulin ester is selected from the group consisting of an inulin butyrate ester, an inulin propionate ester, and a combination thereof 101. The pharmaceutical composition of any one of embodiments 78 to 100, wherein the pharmaceutical composition comprises a nanoemulsion.
102. The pharmaceutical composition of any one of embodiments 78 to 101, wherein the pharmaceutical composition is administered to a subject to treat cancer.
103. The pharmaceutical composition of embodiment 102, wherein the cancer is liver cancer or colon cancer.
104. The pharmaceutical composition of embodiment 103, wherein the cancer is colon cancer and the subject has one or more colon polyps.
105. The pharmaceutical composition of any one of embodiments 78 to 101, wherein the pharmaceutical composition is administered to a subject to treat a metabolic disease.
106. The pharmaceutical composition of embodiment 105, wherein the metabolic disease is selected from the group consisting of non-alcoholic steatohepatitis (NASH), non-alcoholic fatty liver disease (NAFLD), diabetes, obesity, and a combination thereof.
107. A pharmaceutical composition comprising a microRNA (miR) or a mimic thereof and a pharmaceutically acceptable carrier, wherein the miR is selected from the group consisting of miR-22, miR-34a, and a combination thereof.
108. The pharmaceutical composition of embodiment 107, wherein the miR-22 comprises a nucleotide sequence having at least 90% sequence identity to SEQ ID NO:1.
109. The method of embodiment 107, wherein the miR-34a comprises a nucleotide sequence having at least 90% sequence identity to SEQ ID NO:2.
110. The pharmaceutical composition of any one of embodiments 107 to 109, further comprising a starch.
111. The pharmaceutical composition of any one of embodiments 107 to 110, further comprising a probiotic agent and/or a prebiotic agent.
112. The pharmaceutical composition of embodiment 111, wherein the probiotic comprises a bacterium that produces an SCFA.
113. The pharmaceutical composition of embodiment 111, wherein the prebiotic comprises apple pectin, an inulin, or a combination thereof.
114. The pharmaceutical composition of any one of embodiments 107 to 113, further comprising a delivery-enhancing agent.
115. The pharmaceutical composition of embodiment 114, wherein the delivery-enhancing agent is selected from the group consisting of a cyclodextrin, a hepatitis E virus-like particle, an inactivated yeast, an inactivated bacterium, polyvinyl acetate (PVA), an inulin or an ester thereof, and a combination thereof.
116. The pharmaceutical composition of embodiment 115, wherein the inulin ester is selected from the group consisting of an inulin butyrate ester, an inulin propionate ester, and a combination thereof.
117. The pharmaceutical composition of any one of embodiments 107 to 116, wherein the pharmaceutical composition comprises a nanoemulsion.
118. The pharmaceutical composition of any one of embodiments 107 to 117, wherein the pharmaceutical composition is administered to a subject to treat cancer.
119. The pharmaceutical composition of embodiment 118, wherein the cancer is liver cancer or colon cancer.
120. The pharmaceutical composition of embodiment 119, wherein the cancer is colon cancer and the subject has one or more colon polyps.
121. A kit for treating cancer or a metabolic disease in a subject, the kit comprising the pharmaceutical composition of any one of embodiments 78 to 106.
122. The kit of embodiment 121, wherein the cancer is liver or colon cancer.
123. The kit of embodiment 122, wherein the cancer is colon cancer and the subject has one or more colon polyps.
124. The kit of embodiment 121, wherein the metabolic disease is selected from the group consisting of non-alcoholic steatohepatitis (NASH), non-alcoholic fatty liver disease (NAFLD), diabetes, obesity, and a combination thereof.
125. The kit of any one of embodiments 121 to 124, further comprising instructions for use.
126. A kit for treating cancer in a subject, the kit comprising the pharmaceutical composition of any one of embodiments 107 to 120.
127. The kit of embodiment 126, wherein the cancer is liver or colon cancer.
128. The kit of embodiment 127, wherein the cancer is colon cancer and the subject has one or more colon polyps.
129. The kit of any one of embodiments 126 to 128, further comprising instructions for use.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

INFORMAL SEQUENCE LISTING

| SEQ | | |
|---|---|---|
| ID | | Descrip- |
| NO: | Sequence | tion |
| 1 | AAGCUGCCAGUUGAAGAACUGU | miR-22 |
| | | sequence |
| 2 | UGGCAGUGUCUUAGCUGGUUGU | miR-34a |
| | | sequence |
| 3 | GGGTCAGGGCCAGTTCA | DR5 |
| | | motif |
| 4 | TGGACAGAGAGAAGGTCA | ER6 |
| | | motif |
| 5 | TGGCCTGTCACCC | DR1 |
| | | motif |
| 6 | GGGTCAGTGCCCT | IR1 |
| | | motif |
| 7 | TGAACCCTGTGGCCT | DR3 |
| | | motif |
| 8 | GCTGTCATGGTGCCAGAGAGTTGATGGAGCAGCTGGT | Motif |
| | | located |
| | | 4 bp |
| | | away |
| | | from |
| | | IR1 |
| | | motif |

1. A method for treating cancer in a subject, the method comprising administering to the subject a therapeutically effective amount of a retinoid and a histone deacetylase (HDAC) inhibitor.

2. The method of claim 1, wherein the HDAC inhibitor is selected from the group consisting of a short-chain fatty acid (SCFA), suberanilohydroxamic acid (SAHA), and a combination thereof.

3. The method of claim 1, wherein the retinoid is selected from the group consisting of retinoic acid (RA), retinol, retinal, isotretinoin, alltretinoin, etretinate, acitretin, tazarotene, bexarotene, adapalene, seletinoid G, a retinyl ester, fenretinide, derivatives thereof, and a combination thereof.

4. (canceled)

5. (canceled)

6. The method of claim 2, wherein the SCFA is selected from the group consisting of propionate, butyrate, valerate, and a combination thereof.

7-10. (canceled)

11. The method of claim 1, wherein the cancer is liver cancer, colon cancer, or colorectal cancer.

12-25. (canceled)

26. The method of claim 1, wherein the administration of the retinoid and HDAC inhibitor to the subject improves one or more symptoms of cancer in the subject.

27. A method for treating cancer in a subject, the method comprising administering to the subject a therapeutically effective amount of miR-22 or a mimic thereof.

28. The method of claim 27, wherein the miR-22 comprises a nucleotide sequence having at least 90% sequence identity to SEQ ID NO:1.

29. (canceled)

30. The method of claim 27, wherein the miR-22 or mimic thereof is virally expressed.

31. The method of claim 27, wherein the cancer is liver cancer, colon cancer, or colorectal cancer.

32-44. (canceled)

45. The method of claim 27, wherein the administration of the miR-22 or the mimic thereof to the subject improves one or more symptoms of cancer in the subject.

46. A method for treating a metabolic disease in a subject, the method comprising administering to the subject a therapeutically effective amount of a retinoid and a histone deacetylase (HDAC) inhibitor.

47. The method of claim 46, wherein the HDAC inhibitor is selected from the group consisting of a short-chain fatty acid (SCFA), suberanilohydroxamic acid (SAHA), and a combination thereof.

48. The method of claim 46, wherein the retinoid is selected from the group consisting of retinoic acid (RA), retinol, retinal, isotretinoin, alltretinoin, etretinate, acitretin, tazarotene, bexarotene, adapalene, seletinoid G, a retinyl ester, fenretinide, derivatives thereof, and a combination thereof.

49. (canceled)

50. (canceled)

51. The method of claim 47, wherein the SCFA is selected from the group consisting of propionate, butyrate, valerate, and a combination thereof.

52-55. (canceled)

56. The method of claim 46, wherein the metabolic disease is selected from the group consisting of fatty liver disease, non-alcoholic steatohepatitis (NASH), non-alcoholic fatty liver disease (NAFLD), diabetes, obesity, and a combination thereof.

57. (canceled)

58. The method of claim 46, wherein a sample is obtained from the subject.

59-61. (canceled)

62. The method of claim 58, wherein the level of one or more biomarkers is measured in the sample.

63. The method of claim 62, wherein the one or more biomarkers is selected from the group consisting of miR-22, FGF21, FGFR1c, Beta-klotho, blood glucose, aspartate aminotransferase (AST), alanine aminotransferase (ALT), the ratio of AST to ALT, gamma-glutamyl transferase (GGT), the aspartate to platelet ratio index (APRI), alkaline phosphatase (AP), bilirubin, ferritin, alpha-smooth muscle actin (αSMA), procollagen α1 (procol1), transforming growth factor-β (TGFβ), monocyte chemoattractant protein-1 (MCP1), interleukin-10 (IL-1b), tumor necrosis factor alpha (TNFα), connective tissue growth factor (CTGF), platelet derived growth factor receptor beta (PDGFRβ), and a combination thereof.

64-68. (canceled)

69. The method of claim 46, wherein the administration of the retinoid and HDAC inhibitor to the subject improves one or more symptoms of the metabolic disease in the subject.

70. (canceled)

71. The method of claim 1, further comprising administering a probiotic agent and/or a prebiotic agent to the subject.

72. The method of claim 71, wherein the probiotic comprises a bacterium that produces an SCFA that has HDAC inhibitory effect.

73. The method of claim 71, wherein the prebiotic comprises apple pectin, inulin, or a combination thereof.

74-129. (canceled)


SEQUENCE LISTING (11 sequences)

SEQ ID NO: 1 - RNA, 22 nt, Unknown
  Homo sapiens or Mus musculus
aagcugccaguugaagaacugu

SEQ ID NO: 2 - RNA, 22 nt, Unknown
  Homo sapiens or Mus musculus
uggcagugucuuagcugguugu

SEQ ID NO: 3 - DNA, 17 bp, Artificial Sequence
  Synthetic construct
gggtcagggccagttca

SEQ ID NO: 4 - DNA, 18 bp, Artificial Sequence
  Synthetic construct
tggacagagagaaggtca

SEQ ID NO: 5 - DNA, 13 bp, Artificial Sequence
  Synthetic construct
tggcctgtcaccc

SEQ ID NO: 6 - DNA, 13 bp, Artificial Sequence
  Synthetic construct
gggtcagtgccct

SEQ ID NO: 7 - DNA, 15 bp, Artificial Sequence
  Synthetic construct
tgaaccctgtggcct

SEQ ID NO: 8 - DNA, 37 bp, Artificial Sequence
  Synthetic construct
gctgtcatggtgccagagagttgatggagcagctggt

SEQ ID NO: 9 - RNA, 23 nt, Unknown
  Homo sapiens or Mus musculus
uugcacgccaaacucggcagcuu

SEQ ID NO: 10 - RNA, 24 nt, Homo sapiens
acuguuagcuuuaauuggcagcuc

SEQ ID NO: 11 - RNA, 23 nt, Homo sapiens
aggccccgagcucagggcagcug